(19) United States
(12) Patent Application Publication
(10) Pub. No.: US 2021/0330735 A1
(43) Pub. Date: Oct. 28, 2021

(54) COMPOSITIONS AND METHODS FOR THE TREATMENT OF TRAUMATIC OPTIC NEUROPATHY

(71) Applicant: Stealth BioTherapeutics Corp, Monaco (MC)

(72) Inventors: Mark Bamberger, South Glastonbury, CT (US); David T. Tse, Miami, FL (US); Daniel Pelaez, Miami, FL (US)

(21) Appl. No.: 17/260,718

(22) PCT Filed: Jul. 15, 2019
(86) PCT No.: PCT/US2019/041873

§ 371 Date: Jan. 15, 2021

Related U.S. Application Data
(60) Provisional application No. 62/698,742, filed on Jul. 16, 2018.

(51) Int. Cl. A61K 38/07; A61K 38/17; A61K 45/06; A61K 31/4178; A61K 31/05; A61K 31/4409; A61P 27/02

(52) U.S. Cl. CPC A61K 38/07; A61K 38/1793; A61K 45/06; A61P 27/02; A61K 31/05; A61K 31/4409; A61K 31/4178

(57) ABSTRACT

The present disclosure provides novel methods for treating or preventing traumatic optic neuropathy (TON), methods for improving visual function in a subject having TON, methods for promoting retinal ganglion cell (RGC) survival or increasing neurite outgrowth of an RGC, and methods for reducing the risk of having or developing TON in a subject that has experienced a traumatic injury. The methods comprise administering to the subject an effective amount of an aromatic-cationic peptide.

### CROSS-REFERENCE TO RELATED APPLICATIONS

The present application claims the benefit of priority to U.S. Application No. 62/698,742, filed on Jul. 16, 2018, the contents of which are incorporated herein in their entirety.

### TECHNICAL FIELD

The present technology relates generally to compositions and methods for ameliorating or treating traumatic optic neuropathy (TON). Additionally, the present technology relates to administering an effective amount of an aromatic-cationic peptide, such as 2′6′-Dmt-D-Arg-Phe-Lys-NH₂, Phe-D-Arg-Phe-Lys-NH₂, or D-Arg-2′6′-Dmt-Lys-Phe-NH₂, to a subject suffering from or at risk for a TON.

### BACKGROUND

The following description is provided to assist the understanding of the reader. None of the information provided or references cited is admitted to be prior art to the compositions and methods disclosed herein.

Traumatic optic neuropathy (TON) is rare type of optic neuropathy involving loss of vision following damage to the optic nerve secondary to traumatic injury. TON frequently results in profound loss of central vision with the final visual outcome largely dictated by the patient's baseline visual acuities. Other poor prognostic factors include loss of consciousness, no improvement in vision after 48 hours, the absence of visual evoked responses, and evidence of optic canal fractures on neuroimaging. TON most commonly occurs when there is a loss of consciousness associated with multi-system trauma and serious brain injury. Common injuries that result in TON include falls and deceleration injuries from motor vehicle and bicycle accidents.

TON is classified by either the site or mode of traumatic injury. Exemplary sites of injury that lead to TON include trauma to the optic nerve, head trauma, intraorbital injury, intracanalicular injury, and/or intracranial injury. The most common site of injury is the intracanalicular portion of the optic nerve. The mode of traumatic injury in TON is either direct or indirect. In direct TON, optic nerve injuries are caused by trauma to the head or orbit that cross normal tissue planes and disrupt the anatomy and function of the optic nerve. Such direct injuries can be caused, for example, by a bullet or forceps that physically injures the optic nerve. In contrast to direct injuries, indirect injuries transmit force to the optic nerve without transgressing tissue planes. This type of force causes the optic nerve to absorb excess energy at the time of impact. An example of an indirect injury is blunt trauma to the forehead during a motor vehicle accident.

No evidence-based therapies currently exist to effectively treat TON. Common TON treatment options include systemic steroids, surgical decompression of the optic canal, a combination of steroids and surgery, and observation alone. Management of TON using steroids or surgical options is controversial because the efficacy of these treatments remains unclear and their use bears a risk of serious complications and adverse events. For example, adverse events reported with steroid treatment of TON include acute psychosis, acute pancreatitis, gastrointestinal bleeding, wound infections, severe pneumonia, and increased risk of death or severe disability. Adverse events reported with surgical therapies include meningitis and accidental dural exposure. Accordingly, there is a need in the art to develop treatment options for optic neuropathies including TON that exhibit improved efficacy and/or a reduced risk of side effects. The disclosure of the present technology satisfies this need and provides related advantages.

### SUMMARY

In one aspect, the present disclosure provides a method for treating or preventing traumatic optic neuropathy (TON) in a subject in need thereof, comprising administering to the subject a therapeutically effective amount of the peptide D-Arg-2′,6′-Dmt-Lys-Phe-NH₂, or a pharmaceutically acceptable salt thereof.

In some embodiments, the subject has been diagnosed as having TON. In some embodiments, the TON is caused by direct injury or indirect injury to the subject. In some embodiments, the direct or indirect injury is selected from the group consisting of intraorbital injury, intracanalicular injury, intracranial injury, and an injury to the subject's optic nerve.

In some embodiments, the peptide is administered prior to injury. In some embodiments, the peptide is administered immediately following injury. In some embodiments, the peptide is administered about 2 hours or less, about 6 hours or less, about 12 hours or less, or about 24 hours or less following the injury. In some embodiments, the peptide is administered daily for 2 weeks or more. In some embodiments, the peptide is administered daily for 12 weeks or more.

In some embodiments, the treating or preventing comprises the treatment or prevention of one or more signs or symptoms of TON comprising one or more of vision loss, blurred vision, scotoma, decreased color sensation, uveitis, optic neuritis, eye pain, optic nerve avulsion, optic nerve transection, optic nerve sheath hemorrhage, orbital hemorrhage, choroidal rupture, and commotio retinae.

In some embodiments, the subject is a mammal. In some embodiments, the mammalian subject is a human.

In some embodiments, the peptide is administered orally, topically, intranasally, systemically, intravenously, subcutaneously, intraperitoneally, intradermally, intraocularly, ophthalmically, iontophoretically, transmucosally, intravitreally, or intramuscularly.

In some embodiments, the method further comprises separately, sequentially, or simultaneously administering an additional treatment to the subject. In some embodiments, the additional treatment comprises administration of a therapeutic agent. In some embodiments, the therapeutic agent is selected from the group consisting of: TNFα inhibitor, corticosteroid, IL-1R antagonist, resveratrol, potassium channel blocker, and necrostatin-1. In some embodiments, the TNFα inhibitor is etanercept. In some embodiments, the potassium channel blocker is 4-aminopyridine (4-AP). In some embodiments, the additional treatment comprises reducing the core temperature of the subject. In some embodiments, the core temperature of the subject is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%. In some embodiments, hypothermia is induced in the subject. In some embodiments, the combination of peptide and an additional therapeutic treatment has a synergistic effect in the prevention or treatment of TON.

In some embodiments, the pharmaceutically acceptable salt comprises a mono-acetate salt (i.e. a salt comprising one acetate moiety), a bis-acetate salt (i.e. a salt comprising two acetate moieties), a tri-acetate salt, (i.e. a salt comprising three acetate moieties), a tartrate salt, a mono-trifluoroacetate salt (i.e. a salt comprising one trifluoroacetate moiety), a bis-trifluoroacetate salt (i.e. a salt comprising two trifluoroacetate moieties), a tri-trifluoroacetate salt (i.e. a salt comprising three trifluoroacetate moieties), a mono-hydrochloride salt (i.e. a salt comprising one chloride anion such as resulting from or as would be regarded as resulting from inclusion of HCl; a “mono-HCl salt”), a bis-hydrochloride salt (i.e. a salt comprising two chloride anions such as resulting from or as would be regarded as resulting from inclusion of two HCl; a “bis-HCl salt”), a tri-hydrochloride salt (i.e. a salt comprising three chloride anions such as resulting from or as would be regarded as resulting from inclusion of three HCl; a “tri-HCl salt”), a mono-tosylate salt (i.e. a salt comprising one tosylate moiety), a bis-tosylate salt (i.e. a salt comprising two tosylate moieties), or a tri-tosylate salt (i.e. a salt comprising three tosylate moieties). In some embodiments, the peptide that is formulated for administering to a subject is as a tri-HCl salt, a bis-HCl salt, or a mono-HCl salt.

In one aspect, the present disclosure provides a method for improving visual function in a subject having traumatic optic neuropathy (TON), the method comprising administering to the subject a therapeutically effective amount of the peptide D-Arg-2′,6′-Dmt-Lys-Phe-NH₂, or a pharmaceutically acceptable salt thereof.

In some embodiments, the subject has experienced a direct injury or an indirect injury. In some embodiments, the direct or indirect injury is selected from the group consisting of intraorbital injury, intracanalicular injury, intracranial injury, and an injury to the subject's optic nerve.

In some embodiments, the peptide is administered immediately following injury. In some embodiments, the peptide is administered about 2 hours or less, about 6 hours or less, about 12 hours or less, or about 24 hours or less following the injury. In some embodiments, the peptide is administered daily for 2 weeks or more. In some embodiments, the peptide is administered daily for 12 weeks or more.

In some embodiments, the visual function is assessed by one or more of pattern electroretinography (PERG), detection of best corrected visual acuity (BVCA), electroretinography (ERG), and optical coherence tomography (OCT). In some embodiments, the improved visual function comprises improvements in any one or more of visual acuity, BVCA, thickness of the retina as detected by OCT, PERG amplitude, ERG amplitude, ERG latency, vision loss, blurred vision, scotoma, decreased color sensation, uveitis, optic neuritis, eye pain, optic nerve avulsion, optic nerve transection, optic nerve sheath hemorrhage, orbital hemorrhage, choroidal rupture, and commotio retinae compared to an untreated control.

In some embodiments, the subject is a mammal. In some embodiments, the mammalian subject is a human.

In some embodiments, the peptide is administered orally, topically, intranasally, systemically, intravenously, subcutaneously, intraperitoneally, intradermally, intraocularly, ophthalmically, iontophoretically, transmucosally, intravitreally, or intramuscularly.

In some embodiments, the method further comprises separately, sequentially, or simultaneously administering an additional treatment to the subject. In some embodiments, the additional treatment comprises administration of a therapeutic agent. In some embodiments, the therapeutic agent is selected from the group consisting of: TNFα inhibitor, corticosteroid, IL-1R antagonist, resveratrol, potassium-channel blocker, and necrostatin-1. In some embodiments, the TNFα inhibitor is etanercept. In some embodiments, the potassium channel blocker is 4-aminopyridine (4-AP). In some embodiments, the additional treatment comprises reducing the core temperature of the subject. In some embodiments, the core temperature of the subject is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%. In some embodiments, hypothermia is induced in the subject. In some embodiments, the combination of peptide and an additional treatment has a synergistic effect in improving visual function.

In some embodiments, the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride salt, a bis-hydrochloride salt, a tri-hydrochloride salt, a mono-tosylate salt, a bis-tosylate salt, or a tri-tosylate salt. In some embodiments, the peptide that is formulated for administering to a subject is as a tri-HCl salt, a bis-HCl salt, or a mono-HCl salt.

In one aspect, the present disclosure provides a method of promoting retinal ganglion cell (RGC) survival or increasing neurite outgrowth of an RGC comprising contacting an RGC with an effective amount of the peptide D-Arg-2′,6′-Dmt-Lys-Phe-NH₂, or a pharmaceutically acceptable salt thereof. In some embodiments, the RGC is in vitro. In some embodiments, the RGC is in a subject with TON.

In some embodiments, the subject is a mammal. In some embodiments, the mammalian subject is a human.

In some embodiments, the method further comprises separately, sequentially, or simultaneously administering an additional treatment to the subject. In some embodiments, the additional treatment comprises administration of a therapeutic agent. In some embodiments, the therapeutic agent is selected from the group consisting of: TNFα inhibitor, corticosteroid, IL-1R antagonist, resveratrol, potassium-channel blocker, and necrostatin-1. In some embodiments, the TNFα inhibitor is etanercept. In some embodiments, the potassium channel blocker is 4-aminopyridine (4-AP). In some embodiments, the additional treatment comprises reducing the core temperature of the subject. In some embodiments, the core temperature of the subject is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%. In some embodiments, hypothermia is induced in the subject. In some embodiments, the combination of peptide and an additional treatment has a synergistic effect in in promoting RGC survival or increasing neurite outgrowth of an RGC.

In some embodiments, the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride salt, a bis-hydrochloride salt, a tri-hydrochloride salt, a mono-tosylate salt, a bis-tosylate salt or a tri-tosylate salt. In some embodiments, the peptide that is formulated for administering to a subject is as a tri-HCl salt, a bis-HCl salt, or a mono-HCl salt.

In one aspect, the present disclosure provides for the use of a composition in the preparation of a medicament for treating or preventing traumatic optic neuropathy (TON) in a subject in need thereof, wherein the composition comprises a therapeutically effective amount of the peptide D-Arg-2′,6′-Dmt-Lys-Phe-NH₂, or a pharmaceutically acceptable salt thereof.

In some embodiments, the subject has been diagnosed as having TON. In some embodiments, the TON is caused by direct injury or indirect injury to the subject. In some embodiments, the direct or indirect injury is selected from the group consisting of intraorbital injury, intracanalicular injury, intracranial injury, and an injury to the subject's optic nerve.

In some embodiments, the peptide is intended to be administered prior to injury. In some embodiments, the peptide is intended to be administered immediately following injury. In some embodiments, the peptide is intended to be administered about 2 hours or less, about 6 hours or less, about 12 hours or less, or about 24 hours or less following the injury. In some embodiments, the peptide is intended to be administered daily for 2 weeks or more. In some embodiments, the peptide is intended to be administered daily for 12 weeks or more.

In some embodiments, the treating or preventing comprises the treatment or prevention of one or more signs or symptoms of TON comprising one or more of vision loss, blurred vision, scotoma, decreased color sensation, uveitis, optic neuritis, eye pain, optic nerve avulsion, optic nerve transection, optic nerve sheath hemorrhage, orbital hemorrhage, choroidal rupture, and commotio retinae.

In some embodiments, the subject is a mammal. In some embodiments, the mammalian subject is a human.

In some embodiments, the peptide is formulated for administration orally, topically, intranasally, systemically, intravenously, subcutaneously, intraperitoneally, intradermally, intraocularly, ophthalmically, iontophoretically, transmucosally, intravitreally, or intramuscularly.

In some embodiments, the peptide is intended to be separately, sequentially, or simultaneously used with an additional treatment. In some embodiments, the additional treatment comprises use of a therapeutic agent. In some embodiments, the therapeutic agent is selected from the group consisting of: TNFα inhibitor, corticosteroid, IL-1R antagonist, resveratrol, potassium channel blocker, and necrostatin-1. In some embodiments, the TNFα inhibitor is etanercept. In some embodiments, the potassium channel blocker is 4-aminopyridine (4-AP). In some embodiments, the additional treatment comprises reducing the core temperature of the subject. In some embodiments, the core temperature of the subject is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%. In some embodiments, hypothermia is induced in the subject. In some embodiments, the combination of peptide and an additional treatment has a synergistic effect in the prevention or treatment of TON.

In some embodiments, the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride salt, a bis-hydrochloride salt, a tri-hydrochloride salt, a mono-tosylate salt, a bis-tosylate salt or a tri-tosylate salt. In some embodiments, the peptide that is formulated for administering to a subject is as a tri-HCl salt, a bis-HCl salt, or a mono-HCl salt.

In one aspect, the present disclosure provides for the use of a composition in the preparation of a medicament for improving visual function in a subject having traumatic optic neuropathy (TON), wherein the composition comprises a therapeutically effective amount of the peptide D-Arg-2′,6′-Dmt-Lys-Phe-NH₂, or a pharmaceutically acceptable salt thereof.

In some embodiments, the subject has experienced a direct injury or an indirect injury. In some embodiments, the direct or indirect injury is selected from the group consisting of intraorbital injury, intracanalicular injury, intracranial injury, and an injury to the subject's optic nerve.

In some embodiments, the peptide is intended to be administered immediately following injury. In some embodiments, the peptide is intended to be administered about 2 hours or less, about 6 hours or less, about 12 hours or less, or about 24 hours or less following the injury. In some embodiments, the peptide is intended to be administered daily for 2 weeks or more. In some embodiments, the peptide is intended to be administered daily for 12 weeks or more.

In some embodiments, the visual function is assessed by one or more of pattern electroretinography (PERG), detection of best corrected visual acuity (BVCA), electroretinography (ERG), and optical coherence tomography (OCT). In some embodiments, the improved visual function comprises improvements in any one or more of visual acuity, BVCA, thickness of the retina as detected by OCT, PERG amplitude, ERG amplitude, ERG latency, vision loss, blurred vision, scotoma, decreased color sensation, uveitis, optic neuritis, eye pain, optic nerve avulsion, optic nerve transection, optic nerve sheath hemorrhage, orbital hemorrhage, choroidal rupture, and commotio retinae compared to an untreated control.

In some embodiments, the subject is a mammal. In some embodiments, the mammalian subject is a human.

In some embodiments, the peptide is intended to be administered orally, topically, intranasally, systemically, intravenously, subcutaneously, intraperitoneally, intradermally, intraocularly, ophthalmically, iontophoretically, transmucosally, intravitreally, or intramuscularly.

In some embodiments, the peptide is intended to be separately, sequentially, or simultaneously used with an additional treatment. In some embodiments, the additional treatment comprises use of a therapeutic agent. In some embodiments, the therapeutic agent is selected from the group consisting of: TNFα inhibitor, corticosteroid, IL-1R antagonist, resveratrol, potassium channel blocker, and necrostatin-1. In some embodiments, the TNFα inhibitor is etanercept. In some embodiments, the potassium channel blocker is 4-aminopyridine (4-AP). In some embodiments, the additional treatment comprises reducing the core temperature of the subject. In some embodiments, the core temperature of the subject is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%. In some embodiments, hypothermia is induced in the subject. In some embodiments, the combination of peptide and an additional treatment has a synergistic effect in improving visual function.

In some embodiments, the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride salt, a bis-hydrochloride salt, a tri-hydrochloride salt, a mono-tosylate salt, a bis-tosylate salt or a tri-tosylate salt. In some embodiments, the peptide that is formulated for administering to a subject is as a tri-HCl salt, a bis-HCl salt, or a mono-HCl salt.

In one aspect, the present disclosure provides for the use of a composition in the preparation of a medicament for promoting retinal ganglion cell (RGC) survival or increasing neurite outgrowth of an RGC, wherein the composition comprises an effective amount of the peptide D-Arg-2′,6′-Dmt-Lys-Phe-NH₂, or a pharmaceutically acceptable salt thereof. In some embodiments, the RGC is in vitro. In some embodiments, the RGC is in a subject with TON.

In some embodiments, the subject is a mammal. In some embodiments, the mammalian subject is a human.

In some embodiments, the peptide is intended to be separately, sequentially, or simultaneously used an additional treatment. In some embodiments, the additional treatment comprises use of a therapeutic agent. In some embodiments, the therapeutic agent is selected from the group consisting of: TNFα inhibitor, corticosteroid, IL-1R antagonist, resveratrol, potassium channel blocker, and necrostatin-1. In some embodiments, the TNFα inhibitor is etanercept. In some embodiments, the potassium channel blocker is 4-aminopyridine (4-AP). In some embodiments, the additional treatment comprises reducing core temperature. In some embodiments, the core temperature is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%. In some embodiments, hypothermia is induced. In some embodiments, the combination of peptide and an additional treatment has a synergistic effect in in promoting RGC survival or increasing neurite outgrowth of an RGC.

In some embodiments, the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride salt, a bis-hydrochloride salt, a tri-hydrochloride salt, a mono-tosylate salt, a bis-tosylate salt or a tri-tosylate salt. In some embodiments, the peptide that is formulated for administering to a subject is as a tri-HCl salt, a bis-HCl salt, or a mono-HCl salt.

In one aspect, the present disclosure provides a peptide D-Arg-2′,6′-Dmt-Lys-Phe-NH₂, or a pharmaceutically acceptable salt thereof, for use in treating or preventing traumatic optic neuropathy (TON) in a subject in need thereof.

In some embodiments, the subject has been diagnosed as having TON. In some embodiments, the TON is caused by direct injury or indirect injury to the subject. In some embodiments, the direct or indirect injury is selected from the group consisting of intraorbital injury, intracanalicular injury, intracranial injury, and an injury to the subject's optic nerve.

In some embodiments, the peptide is intended to be administered prior to injury. In some embodiments, the peptide is intended to be administered immediately following injury. In some embodiments, the peptide is intended to be administered about 2 hours or less, about 6 hours or less, about 12 hours or less, or about 24 hours or less following the injury. In some embodiments, the peptide is intended to be administered daily for 2 weeks or more. In some embodiments, the peptide is intended to be administered daily for 12 weeks or more.

In some embodiments, the treating or preventing comprises the treatment or prevention of one or more signs or symptoms of TON comprising one or more of vision loss, blurred vision, scotoma, decreased color sensation, uveitis, optic neuritis, eye pain, optic nerve avulsion, optic nerve transection, optic nerve sheath hemorrhage, orbital hemorrhage, choroidal rupture, and commotio retinae.

In some embodiments, the subject is a mammal. In some embodiments, the mammalian subject is a human.

In some embodiments, the peptide for use is formulated for administration orally, topically, intranasally, systemically, intravenously, subcutaneously, intraperitoneally, intradermally, intraocularly, ophthalmically, iontophoretically, transmucosally, intravitreally, or intramuscularly. In some embodiments, the peptide for use is intended to be separately, sequentially, or simultaneously used with an additional treatment. In some embodiments, the additional treatment comprises use of a therapeutic agent. In some embodiments, the therapeutic agent is selected from the group consisting of: TNFα inhibitor, corticosteroid, IL-1R antagonist, resveratrol, potassium channel blocker, and necrostatin-1. In some embodiments, the TNFα inhibitor is etanercept. In some embodiments, the potassium channel blocker is 4-aminopyridine (4-AP). In some embodiments, the additional treatment comprises reducing the core temperature of the subject. In some embodiments, the core temperature of the subject is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%. In some embodiments, hypothermia is induced in the subject. In some embodiments, the combination of peptide and an additional treatment has a synergistic effect in the prevention or treatment of TON.

In some embodiments, the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride salt, a bis-hydrochloride salt, a tri-hydrochloride salt, a mono-tosylate salt, a bis-tosylate salt or a tri-tosylate salt. In some embodiments, the peptide that is formulated for administering to a subject is as a tri-HCl salt, a bis-HCl salt, or a mono-HCl salt.

In one aspect, the present disclosure provides a peptide D-Arg-2′,6′-Dmt-Lys-Phe-NH₂, or a pharmaceutically acceptable salt thereof, for use in improving visual function in a subject having traumatic optic neuropathy (TON).

In some embodiments, the subject has experienced a direct injury or an indirect injury. In some embodiments, the direct or indirect injury is selected from the group consisting of intraorbital injury, intracanalicular injury, intracranial injury, and an injury to the subject's optic nerve. In some embodiments, the peptide is intended to be administered immediately following injury. In some embodiments, the peptide is intended to be administered about 2 hours or less, about 6 hours or less, about 12 hours or less, or about 24 hours or less following the injury. In some embodiments, the peptide is intended to be administered daily for 2 weeks or more. In some embodiments, the peptide is intended to be administered daily for 12 weeks or more.

In some embodiments, the visual function is assessed by one or more of pattern electroretinography (PERG), detection of best corrected visual acuity (BVCA), electroretinography (ERG), and optical coherence tomography (OCT). In some embodiments, the improved visual function comprises improvements in any one or more of visual acuity, BVCA, thickness of the retina as detected by OCT, PERG amplitude, ERG amplitude, ERG latency, vision loss, blurred vision, scotoma, decreased color sensation, uveitis, optic neuritis, eye pain, optic nerve avulsion, optic nerve transection, optic nerve sheath hemorrhage, orbital hemorrhage, choroidal rupture, and commotio retinae compared to an untreated control.

In some embodiments, the subject is a mammal. In some embodiments, the mammalian subject is a human.

In some embodiments, the peptide is intended to be administered orally, topically, intranasally, systemically, intravenously, subcutaneously, intraperitoneally, intradermally, intraocularly, ophthalmically, iontophoretically, transmucosally, intravitreally, or intramuscularly.

In some embodiments, the peptide is intended to be separately, sequentially, or simultaneously used with an additional treatment. In some embodiments, the additional treatment comprises use of a therapeutic agent. In some embodiments, the therapeutic agent is selected from the group consisting of: TNFα inhibitor, corticosteroid, IL-1R antagonist, resveratrol, potassium channel blocker, and necrostatin-1. In some embodiments, the TNFα inhibitor is etanercept. In some embodiments, the potassium channel blocker is 4-aminopyridine (4-AP). In some embodiments, the additional treatment comprises reducing the core temperature of the subject. In some embodiments, the core temperature of the subject is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%. In some embodiments, hypothermia is induced in the subject. In some embodiments, the combination of peptide and an additional treatment has a synergistic effect in improving visual function.

In some embodiments, the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride salt, a bis-hydrochloride salt, a tri-hydrochloride salt, a mono-tosylate salt, a bis-tosylate salt or a tri-tosylate salt. In some embodiments, the peptide that is formulated for administering to a subject is as a tri-HCl salt, a bis-HCl salt, or a mono-HCl salt.

In one aspect, the present disclosure provides a peptide D-Arg-2′,6′-Dmt-Lys-Phe-NH₂, or a pharmaceutically acceptable salt thereof, for use in promoting retinal ganglion cell (RGC) survival or increasing neurite outgrowth of an RGC. In some embodiments, the RGC is in vitro. In some embodiments, the RGC is in a subject with TON.

In some embodiments, the subject is a mammal. In some embodiments, the mammalian subject is a human.

In some embodiments, the peptide is intended to be separately, sequentially, or simultaneously used an additional treatment. In some embodiments, the additional treatment comprises use of a therapeutic agent. In some embodiments, the therapeutic agent is selected from the group consisting of: TNFα inhibitor, corticosteroid, IL-1R antagonist, resveratrol, potassium channel blocker, and necrostatin-1. In some embodiments, the TNFα inhibitor is etanercept. In some embodiments, the potassium channel blocker is 4-aminopyridine (4-AP). In some embodiments, the additional treatment comprises reducing core temperature. In some embodiments, the core temperature is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%. In some embodiments, hypothermia is induced. In some embodiments, the combination of peptide and an additional treatment has a synergistic effect in in promoting RGC survival or increasing neurite outgrowth of an RGC.

In some embodiments, the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride salt, a bis-hydrochloride salt, a tri-hydrochloride salt, a mono-tosylate salt, a bis-tosylate salt or a tri-tosylate salt. In some embodiments, the peptide that is formulated for administering to a subject is as a tri-HCl salt, a bis-HCl salt, or a mono-HCl salt.

In one aspect, the present disclosure provides a method for reducing the risk of TON in a subject that has experienced a traumatic injury, the method comprising administering to the subject a therapeutically effective amount of the peptide D-Arg-2′,6′-Dmt-Lys-Phe-NH₂, or a pharmaceutically acceptable salt thereof.

In some embodiments, the traumatic injury is a direct injury or an indirect injury. In some embodiments, the direct or indirect injury is selected from the group consisting of intraorbital injury, intracanalicular injury, intracranial injury, and an injury to the subject's optic nerve.

In some embodiments, the peptide is administered immediately following the traumatic injury. In some embodiments, the peptide is administered about 2 hours or less, about 6 hours or less, about 12 hours or less, or about 24 hours or less following the traumatic injury. In some embodiments, the peptide is administered daily for 2 weeks or more. In some embodiments, the peptide is administered daily for 12 weeks or more.

In some embodiments, the subject is a mammal. In some embodiments, the mammalian subject is a human.

In some embodiments, the peptide is administered orally, topically, intranasally, systemically, intravenously, subcutaneously, intraperitoneally, intradermally, intraocularly, ophthalmically, iontophoretically, transmucosally, intravitreally, or intramuscularly.

In some embodiments, the method further comprises separately, sequentially, or simultaneously administering an additional treatment to the subject. In some embodiments, the additional treatment comprises administration of a therapeutic agent. In some embodiments, the therapeutic agent is selected from the group consisting of: TNFα inhibitor, corticosteroid, IL-1R antagonist, resveratrol, potassium channel blocker, and necrostatin-1. In some embodiments, the TNFα inhibitor is etanercept. In some embodiments, the potassium channel blocker is 4-aminopyridine (4-AP). In some embodiments, the additional treatment comprises reducing the core temperature of the subject. In some embodiments, the core temperature of the subject is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%. In some embodiments, hypothermia is induced in the subject. In some embodiments, the combination of peptide and an additional treatment has a synergistic effect in the prevention or treatment of TON.

In some embodiments, the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride salt, a bis-hydrochloride salt, a tri-hydrochloride salt, a mono-tosylate salt, a bis-tosylate salt or a tri-tosylate salt. In some embodiments, the peptide that is formulated for administering to a subject is as a tri-HCl salt, a bis-HCl salt, or a mono-HCl salt.

The present technology relates generally to novel methods for treating or preventing optic neuropathies, methods for improving visual function in a subject having an optic neuropathy, methods for promoting retinal ganglion cell (RGC) survival or increasing neurite outgrowth of an RGC, methods for reducing the risk of having or developing traumatic optic neuropathy (TON) in a subject that has experienced a traumatic injury, uses of a composition in the preparation of a medicament for treating or preventing optic neuropathy in a subject in need thereof, uses of a composition in the preparation of a medicament for improving visual function in a subject having optic neuropathy, uses of a composition in the preparation of a medicament for promoting retinal ganglion cell (RGC) survival or increasing neurite outgrowth of an RGC, aromatic-cationic peptides for use in treating or preventing optic neuropathy in a subject in need thereof, aromatic-cationic peptides for use in improving visual function in a subject having optic neuropathy, and aromatic-cationic peptides for use in promoting retinal ganglion cell (RGC) survival or increasing neurite outgrowth of an RGC.

### BRIEF DESCRIPTION OF DRAWINGS

FIGS. 1A-1B. Retinal ganglion cell (RGC) survival two weeks following injury. FIG. 1A depicts the percent RGC survival in a sonication-induced-traumatic optic neuropathy (SI-TON) model treated with (1) PBS, (2) etanercept, or (3) D-Arg-2′6′-Dmt-Lys-Phe-NH₂. FIG. 1B depicts the percent RGC survival in an optic nerve crush-traumatic optic neuropathy (ONC-TON) model treated with (1) PBS, (2) etanercept, or (3) D-Arg-2′6′-Dmt-Lys-Phe-NH₂. (FIG. 1A: Group 1=76.66±2.32%; Group 2=97.08±2.21% (p=0.000216); Group 3=93.70±1.58% (p=0.000304); FIG. 1B: Group 1=24.71±1.21%; Group 2=48.94±1.94% (p=1.97×10⁻⁵); Group 3=51.13±1.47% (p=1.61×10⁻⁵)).

FIG. 2. PERG response of ultrasound induced optic nerve trauma at 1 week and 2 weeks following injury. The bars depict the PERG amplitude (uV) of: (1) an OD control (TON OS-1 week); (2) an OD control (TON OS-1 week)+D-Arg-2′6′-Dmt-Lys-Phe-NH₂; (3) an OD control (TON OS-2 weeks); (4) an OD control (TON OS-2 weeks)+D-Arg-2′6′-Dmt-Lys-Phe-NH₂; (5) OS (TON OS-1 week); (6) OS (TON OS-1 week)+D-Arg-2′6′-Dmt-Lys-Phe-NH₂; (7) OS (TON OS-2 weeks); and (8) OS (TON OS-2 weeks)+D-Arg-2′6′-Dmt-Lys-Phe-NH₂.

FIG. 3. PERG response of crush induced optic nerve trauma at 1 week following injury. The bars depict the PERG amplitude (uV) of: (1) an OD control (Crush OS-1 week); (2) an OD control (Crush OS-1 week)+D-Arg-2′6′-Dmt-Lys-Phe-NH₂; (3) OS (Crush OS-1 week); and (4) OS (Crush OS-1 week)+D-Arg-2′6′-Dmt-Lys-Phe-NH₂.

FIG. 4. Schematic representation of the sequential administration study of D-Arg-2′6′-Dmt-Lys-Phe-NH₂ (MTP-131) with Etanercept (Enbrel™), 4-aminopyridine (4-AP) or PBS. Sequences of administration were categorized as followed: Group 1, Etanercept, D-Arg-2′6′-Dmt-Lys-Phe-NH₂, and 4-AP; Group 2, Etanercept, D-Arg-2′6′-Dmt-Lys-Phe-NH₂, and PBS; Group 3, D-Arg-2′6′-Dmt-Lys-Phe-NH₂, Etanercept, and 4-AP; Group 4, D-Arg-2′6′-Dmt-Lys-Phe-NH₂, Etanercept, and PBS; Group 5, PBS, PBS, and 4-AP; Group 6, PBS only.

FIGS. 5A-5D. Effects of sequential administration of D-Arg-2′6′-Dmt-Lys-Phe-NH₂ with Etanercept on visual function loss following US-TON (SI-TON). FIG. 5A depicts scatter plots illustrating a timeline of visual function as assessed by PERG amplitude following US-TON induction and sequential administration of D-Arg-2′6′-Dmt-Lys-Phe-NH₂ with Etanercept. FIG. 5B depicts a bar graph quantifying the effectiveness of the sequential administration of Etanercept followed by D-Arg-2′6′-Dmt-Lys-Phe-NH₂ at preventing loss of visual function two weeks after treatments. FIG. 5C depicts a bar graph quantifying the effectiveness of the sequential administration of Etanercept followed by D-Arg-2′6′-Dmt-Lys-Phe-NH₂ at preventing loss of visual function four weeks after treatment. FIG. 5D depicts a bar graph quantifying the thickness of the Retinal Nerve Fiber Layer (RNFL) and Inner Plexiform Layer (IPL) four weeks after treatments.

FIGS. 6A-6B. Retinal ganglion cells (RGC) survival four weeks following SI-TON-induced injury in mice. FIG. 6A depicts the percentage of RGC survival following treatment with (1) PBS; (2) sequential administration of intravitreal D-Arg-2′6′-Dmt-Lys-Phe-NH₂ 15 minutes post-injury, followed by subcutaneous Etanercept injection for 3 days, and subcutaneous D-Arg-2′6′-Dmt-Lys-Phe-NH₂ injection for another three days post injury; (3) subcutaneous Etanercept injection for 3 days, and subcutaneous D-Arg-2′6′-Dmt-Lys-Phe-NH₂ injection for another three days; (4) Etanercept injection alone; and (5) subcutaneous D-Arg-2′6′-Dmt-Lys-Phe-NH₂ injection alone. FIG. 6B depicts immunohistochemical staining of neuronal marker Beta3-tubulin (TUBB3). TUBB3 marks neuoronal tubulin and FIG. 6B depicts RGC staining and axonal projection in the retinal nerve fiber layer (RNFL) in the various treatment cohorts. (FIG. 6A: Group 2: p=5.7×10⁻⁷ (to Group 1); p=0.0052 (to Group 3); Group 3: p=0.0272; Group 4: p=0.000216; Group 5: p=0.000304).

FIGS. 7A-7B. Acute intravitreal administration of D-Arg-2′6′-Dmt-Lys-Phe-NH₂ is safe and effective. FIG. 7A depicts a graph illustrating the effect of D-Arg-2′6′-Dmt-Lys-Phe-NH₂ acute intravitreal administration in the left eye on visual function pre- and post-injury. The graph quantifies the electrical activity of RGCs from (1) OS_control_Uninjured (baseline); (2) OS_D-Arg-2′6′-Dmt-Lys-Phe-NH₂_Uninjured (baseline); (3) OS_control_TON; (4) OS_D-Arg-2′6′-Dmt-Lys-Phe-NH₂_TON. FIG. 7B depicts a graph illustrating the effectiveness of acute intravitreal administration of D-Arg-2′6′-Dmt-Lys-Phe-NH₂ in the left eye at improving visual function post-injury when compared to subcutaneous injection. The graph quantifies the electrical activity of RGCs from (1) OS_control_Uninjured; (2) OS_D-Arg-2′6′-Dmt-Lys-Phe-NH₂ subcutaneous injection_Uninjured; (3) OS_D-Arg-2′6′-Dmt-Lys-Phe-NH₂ intravitreal injection_Uninjured; (4) OS_control_TON; (5) OS_D-Arg-2′6′-Dmt-Lys-Phe-NH₂ subcutaneous injection_TON, and (6) OS_D-Arg-2′6′-Dmt-Lys-Phe-NH₂ intravitreal injection_TON. Groups 1, 2, 3, and 4 of FIG. 7A are the same as Groups 1, 3, 4, and 6, respectively, of FIG. 7B. (p=0.0042).

### DETAILED DESCRIPTION

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

In practicing the present technology, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, N Y, 1987); and Meth. Enzymol., Vols. 154 and 155, Wu & Grossman, and Wu, Eds., respectively.

As used in this specification and the appended claims, the singular forms “a”, “an” and “the” include plural referents unless the content clearly dictates otherwise. For example, reference to “a cell” includes a combination of two or more cells, and the like.

As used herein, the “administration” of an agent, drug, or peptide to a subject includes any route of introducing or delivering to a subject a compound to perform its intended function. Administration can be carried out by any suitable route, including orally, intranasally, intraocularly, ophthalmically, parenterally (intravenously, intramuscularly, intraperitoneally, or subcutaneously), intravitreally, or topically. Administration includes self-administration and the administration by another.

As used herein, the term “amino acid” includes naturally-occurring amino acids and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally-occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally-occurring amino acid. Amino acid mimetics refers to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein, the term “effective amount” refers to a quantity sufficient to achieve a desired therapeutic and/or prophylactic effect, e.g., an amount which results in partial or full amelioration of one or more symptoms of TON. In the context of therapeutic or prophylactic applications, in some embodiments, the amount of a composition administered to the subject will depend on the type, degree, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as a mono, bis or tri-acetate salt, a tartrate salt, a fumerate salt, a mono, bis or tri-trifluoroacetate salt, a mono, bis, or tri-HCl salt, or a mono, bis or tri-tosylate salt, may be administered to a subject having one or more signs, symptoms, or risk factors of TON, including, but not limited to, traumatic injury, vision loss, blurred vision, RGC damage, scotoma, decreased color sensation, uveitis, optic neuritis, eye pain, optic nerve avulsion, optic nerve transection, optic nerve sheath hemorrhage, orbital hemorrhage, choroidal rupture, and commotio retinae.

As used herein, “isolated” or “purified” polypeptide or peptide refers to a polypeptide or peptide that is substantially free of cellular material or other contaminating polypeptides from the cell or tissue source from which the agent is derived, or substantially free from chemical precursors or other chemicals when chemically synthesized. For example, an isolated aromatic-cationic peptide would be free of materials that would interfere with diagnostic or therapeutic uses of the agent. Such interfering materials may include enzymes, hormones and other proteinaceous and nonproteinaceous solutes.

As used herein, the terms “polypeptide,” “polyamino acid,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, “prevention” or “preventing” of a disorder or condition refers to a compound that, in a statistical sample, reduces the occurrence of the disorder or condition in the treated sample relative to an untreated control sample, or delays the onset of one or more symptoms of the disorder or condition relative to the untreated control sample. As used herein, preventing TON, includes preventing or delaying the initiation of symptoms of TON. As used herein, prevention of TON also includes preventing a recurrence of one or more signs or symptoms of TON.

As used herein, the terms “subject” and “patient” are used interchangeably.

In the context of therapeutic use or administration, the term “separate” or “separately” refers to an administration of at least two active ingredients by different routes, formulations, and/or pharmaceutical compositions.

The term “simultaneous” therapeutic use refers to administration of at least two active ingredients at the same time or at substantially the same time. In some embodiments, simultaneous administration includes but is not limited to administration of a single composition or formulation comprising at least two active ingredients, co-administration of at least two separate active ingredients by the same route, and co-administration of at least two separate active ingredients by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, a “synergistic therapeutic effect” refers to a greater-than-additive therapeutic effect which is produced by a combination of at least two agents, and which exceeds that which would otherwise result from the individual administration of the agents. For example, lower doses of one or more agents may be used in treating optic neuropathies such as TON resulting in increased therapeutic efficacy and decreased side-effects.

As used herein, a “traumatic injury” is any injury that has the potential to cause serious tissue damage, disability, prolonged disability, and/or death in the subject that has experienced the injury. Traumatic injuries include, but are not limited to, blunt injuries, penetrating injuries, falls, motor vehicle collisions, stabbing wounds, and gunshot wounds. In the context of the optic nerve, traumatic injuries can be categorized as direct injuries and indirect injuries. Nonlimiting examples of such optic nerve injuries include intraorbital injury, intracanalicular injury, intracranial injury, and an injury to the subject's optic nerve head.

As used herein, a “traumatic optic neuropathy” or “TON” refers to optic neuropathy caused by traumatic injury. In some cases, the injury is direct or indirect. Exemplary features of TON include, but are not limited to: unilateral or bilateral ocular involvement, relative afferent papillary defect except in cases of symmetric bilateral TON, variable loss of visual acuity ranging from normal to no light perception, impairment of color vision, variable visual field defects, abnormal appearance of optic disc, and/or development of optic atrophy (typically within six weeks following injury). Nonlimiting examples of TON symptoms vision loss, blurred vision, RGC damage, scotoma, decreased color sensation, uveitis, optic neuritis, eye pain, optic nerve avulsion, optic nerve transection, optic nerve sheath hemorrhage, orbital hemorrhage, choroidal rupture, and commotio retinae.

As used herein, the terms “treating” or “treatment” or “alleviation” refers to therapeutic treatment, wherein the object is to reduce, alleviate or slow down the progression or advancement of, and/or reverse the progression of the targeted pathological condition or disorder. A subject is successfully “treated” for an optic neuropathy, if, after receiving a therapeutic amount of the aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof, such as an acetate salt, a tartrate salt, a trifluoroacetate salt, a chloride salt, a salt of three hydrochlorides (a “tris-HCl salt”), a salt of two hydrochlorides (a “bis-HCl salt”), a salt of one hydrochloride (a “mono-HCl salt”), or a tosylate salt, according to the methods described herein, the subject shows observable and/or measurable reduction in or absence of one or more signs and symptoms of the optic neuropathy. For example, in TON, such signs and symptoms include, but are not limited to, vision loss, blurred vision, RGC damage, scotoma, decreased color sensation, uveitis, optic neuritis, eye pain, optic nerve avulsion, optic nerve transection, optic nerve sheath hemorrhage, orbital hemorrhage, choroidal rupture, and commotio retinae.

It is also to be appreciated that the various modes of treatment or prevention of medical conditions as described herein are intended to mean “substantial,” which includes total but also less than total treatment or prevention, and wherein some biologically or medically relevant result is achieved.

### Traumatic Optic Neuropathy and Retinal Ganglion Cells

Optic neuropathy caused by traumatic injury (i.e., TON) is a clinical diagnosis supported by a history of trauma to the head or face. In some cases, the injury is direct or indirect. Exemplary features of TON include, but are not limited to: unilateral or bilateral ocular involvement, relative afferent papillary defect except in cases of symmetric bilateral TON, variable loss of visual acuity ranging from normal to no light perception, impairment of color vision, variable visual field defects, abnormal appearance of optic disc, and/or development of optic atrophy (typically within six weeks following injury). Approximately 40% to 60% of TON patients present with severe visual loss of light perception. Direct TON frequently causes severe and immediate visual loss with little likelihood for recovery. Indirect TON can be associated with delayed visual loss secondary to the development of an optic nerve sheath hematoma.

By way of example, but not by way of limitation, in some embodiments, symptoms of TON include, but are not limited to, vision loss, blurred vision, retinal ganglion cell damage, scotoma, decreased color sensation, uveitis, optic neuritis, eye pain, optic nerve avulsion, optic nerve transection, optic nerve sheath hemorrhage, orbital hemorrhage, choroidal rupture, and commotio retinae.

The optic nerve contains axons of nerve cells that emerge from the retina, leave the eye at the optic disc, and enter the visual cortex where input from the eye is processed into vision. Optic nerve fibers are derived from the retinal ganglion cells of the inner retina. TON may involve damage to the optic nerve including, but not limited to, damage to retinal ganglion cells (e.g., RGC death and/or RGC axonal damage). A retinal ganglion cell (RGC) is a type of neuron located near the inner surface (the ganglion cell layer) of the retina of the eye. During development, secreted guidance molecules along with signals from extracellular matrix and the vasculature guide RGC positioning, for example, around the fovea, and axon outgrowth. RGCs receive visual information from photoreceptors via two intermediate neuron types: bipolar cells and retina amacrine cells. Retina amacrine cells, particularly narrow field cells, are important for creating functional subunits within the ganglion cell layer and making it so that ganglion cells can observe a small dot moving a small distance. Retinal ganglion cells collectively transmit image-forming and non-image forming visual information from the retina in the form of action potential to several regions in the thalamus, hypothalamus, and mesencephalon, or midbrain.

RGCs are classified into the three major subtypes of RGCs, namely midget, parasol and small bistratified ganglion cells, which are thought to contribute to the parvocellular, magnocellular and koniocellular pathways, respectively. These distinct RGC populations and their associated pathways can be tested by modifying standard psychophysical measures. In general, the processing of high spatial frequency information has been linked with the parvocellular pathway whereas high temporal frequency information is thought to be integrated by the magnocellular pathway. Red-green processing and blue-yellow processing have been linked with the parvocellular and koniocellular pathways, respectively.

### Aromatic-Cationic Peptides

In some embodiments, the aromatic-cationic peptides of the present technology are water-soluble, highly polar, and can readily penetrate cell membranes.

The maximum number of amino acids present in the aromatic-cationic peptides of the present technology is about twenty amino acids covalently joined by peptide bonds. In some embodiments, the total number of amino acids is about twelve. In some embodiments, the total number of amino acids is about nine. In some embodiments, the total number of amino acids is about six. In some embodiments, the total number of amino acids is four. In some embodiments, the total number of amino acids is three.

In some aspects, the present technology provides an aromatic-cationic peptide or a pharmaceutically acceptable salt thereof such as an mono, bis or tri-acetate salt, a tartrate salt, a fumarate salt, a mono, bis, or tri-HCl salt, a mono, bis, or tri-tosylate salt, or a mono, bis, or tri-trifluoroacetate salt. In some embodiments, the peptide comprises at least one net positive charge; a minimum of three amino acids; a maximum of about twenty amino acids; a relationship between the minimum number of net positive charges (pₘ) and the total number of amino acid residues (r) wherein 3pₘ is the largest number that is less than or equal to r+1; and a relationship between the minimum number of aromatic groups (a) and the total number of net positive charges (pₜ) wherein 2a is the largest number that is less than or equal to pₜ+1, except that when a is 1, pₜ may also be 1.

In some embodiments, the peptide is defined by Formula I:

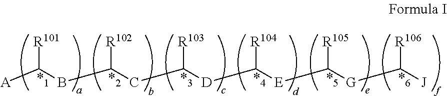

wherein:

one of A and J is

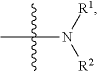

and the other of A and J is

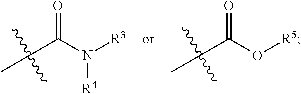

B, C, D, E, and G are each

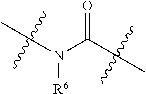

or B, C, D, E, and G are each

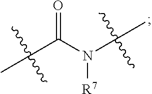

with the proviso that when
   f is 0 and J is not a terminal group, the terminal group is one of G, E, D or C, such that
   one of A and the terminal group is

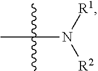

and
the other of A and the terminal group is

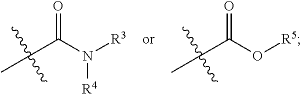

R¹⁰¹ is

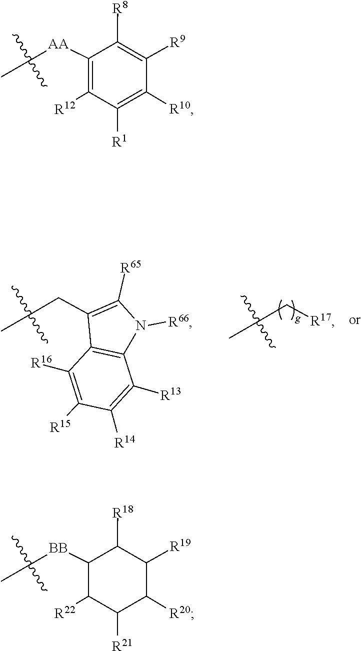

R¹⁰² is

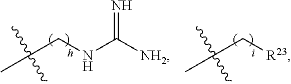

or hydrogen;

R¹⁰³ is

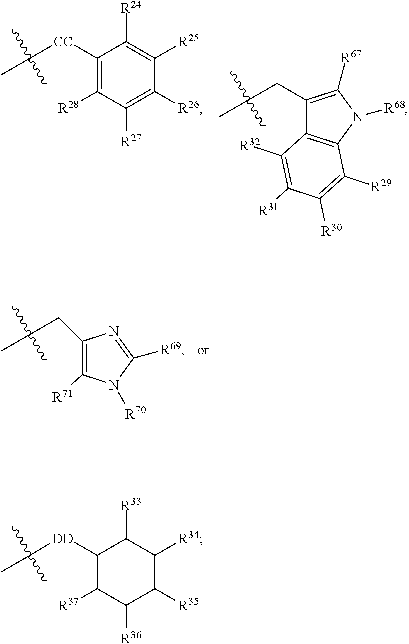

R¹⁰⁴ is

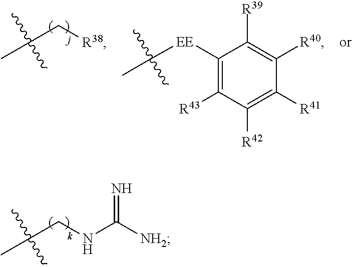

R¹⁰⁵ is

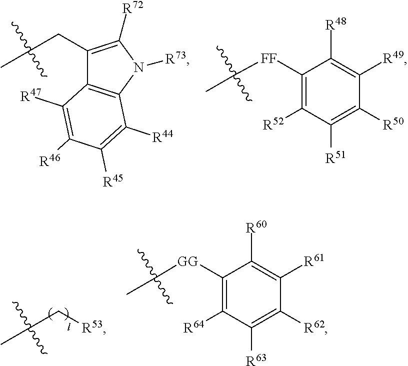

or hydrogen;

R¹⁰⁶ is

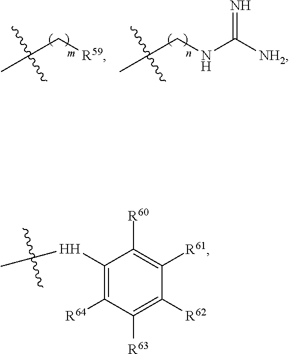

or hydrogen;
provided that when R¹⁰², R¹⁰⁴, and R¹⁰⁶ are identical, then R¹⁰¹, R¹⁰³, and R¹⁰⁵ are not identical;
wherein
   [symbol] , [symbol] , and [symbol] each independently indicate carbon stereocenters, an absolute configuration of each of which is independently at each occurrence R or S;
   when R¹⁰² is not hydrogen, then [symbol] indicates a carbon stereocenter with an absolute configuration of R or S;
   when R¹⁰⁵ is not hydrogen, then [symbol] indicates a carbon stereocenter with an absolute configuration of R or S;
   when R¹⁰⁶ is not hydrogen, then [symbol] indicates a carbon stereocenter with an absolute configuration of R or S;
   R¹ and R², are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, heteroaryl, or amino protecting group; or R¹ and R² together form a 3, 4, 5, 6, 7, or 8 membered substituted or unsubstituted heterocycyl ring;
   R³, R⁴, and R⁵ are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, or heteroaryl; or R³ and R⁴ together form a 3, 4, 5, 6, 7, or 8 membered substituted or unsubstituted heterocycyl ring;
   R⁶ and R⁷ at each occurrence are independently a hydrogen or substituted or unsubstituted C₁-C₆ alkyl group;
   R⁸, R⁹, R¹⁰, R¹¹, R¹², R¹³, R¹⁴, R¹⁵, R¹⁶, R¹⁸, R¹⁹, R²⁰, R²¹, R²², R²⁴, R²⁵, R²⁶, R²⁷, R²⁸, R²⁹, R³⁰, R³¹, R³², R³³, R³⁴, R³⁵, R³⁶, R³⁷, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵⁴, R⁵⁵, R⁵⁶, R⁵⁷, R⁵⁸, R⁶⁰, R⁶¹, R⁶², R⁶³, R⁶⁴, R⁶⁵, R⁶⁷, R⁶⁹, R⁷¹ and R⁷² are each independently a hydrogen, amino, amido, —NO₂, —CN, —OR^{a}, —SR^{a}, —NR^{a}R^{a}, —F, —Cl, —Br, —I, or a substituted or unsubstituted C₁-C₆ alkyl, C₁-C₆ alkoxy, —C(O)-alkyl, —C(O)-aryl, —C(O)-aralkyl, —C(O)₂R^{a}, C₁-C₄ alkylamino, C₁-C₄ dialkylamino, or perhaloalkyl group;
   R⁶⁶, R⁶⁸, R⁷⁰, and R⁷³ are each independently a hydrogen or substituted or unsubstituted C₁-C₆ alkyl group;
   R¹⁷, R²³, R³⁸, R⁵³, and R⁵⁹ are each independently a hydrogen, —OR^{a}, —SR^{a}, —NR^{a}R^{a}, —NR^{a}R^{b}, —CO₂R^{a}, —(CO)NR^{a}R^{a}, —NR^{a}(CO)R^{a}, —NR^{a}C(NH)NH₂, —NR^{a}-dansyl, or a substituted or unsubstituted alkyl, aryl, or aralkyl group;
   AA, BB, CC, DD, EE, FF, GG, and HH are each independently absent, —NH(CO)—, —CH₂—, or —CH₂CH₂—;
   R^{a} at each occurrence is independently a hydrogen or a substituted or unsubstituted C₁-C₆ alkyl group;
   R^{b} at each occurrence is independently a C₁-C₆ alkylene-NR^{a}-dansyl or C₁-C₆ alkylene-NR^{a}-anthraniloyl group;
   a, b, c, d, e, and f are each independently 0 or 1, with the proviso that a+b+c+d+e+f≥2;
   g, h, k, m, and n are each independently 1, 2, 3, 4, or 5; and
   i, j, and l are each independently 1, 2, 3, 4, or 5;
   optionally provided that
      when i is 4 and R²³ is —SR^{a}, or j is 4 and R³⁸ is —SR^{a}, or l is 4 and R⁵³ is —SR^{a}, then the R^{a} of the —SR^{a} is a substituted or unsubstituted C₁-C₆ alkyl group;
      when J is —NH₂, b and dare 0, a, c, e, f are 1, then R¹⁰³ is

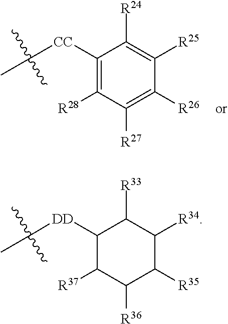

In some embodiments of peptides of Formula I,
R¹, R², R³, R⁴, and R⁵ are each independently a hydrogen or substituted or unsubstituted C₁-C₆ alkyl group;
R⁶ and R⁷ at each occurrence are independently a hydrogen or methyl group;
R⁸, R¹², R¹⁸, R²², R²⁴, R²⁸, R³³, R³⁷, R³⁹, R⁴³, R⁴⁸, R⁵², R⁵⁴, R⁵⁸, R⁶⁰, and R⁶⁴ are each independently a hydrogen or methyl group;
R¹⁰, R²⁰, R²⁶, R³⁵, R⁴¹, R⁵⁰, R⁵⁶, and R⁶² are each independently a hydrogen or —OR^{a};
R⁹, R¹¹, R¹⁹, R²¹, R²⁵, R²⁷, R³⁴, R³⁶, R⁴⁰, R⁴², R⁴⁹, R⁵¹, R⁵⁵, R⁵⁷, R⁶¹, R⁶³, R⁶⁵, R⁶⁶, R⁶⁷, R⁶⁸, R⁶⁹, R⁷⁰, R⁷¹, R⁷², and R⁷³ are each a hydrogen;
R¹⁷, R²³, R³⁸, R⁵³, and R⁵⁹ are each independently a hydrogen, —OH, —SH, —SCH₃, —NH₂, —NHR^{b}, —CO₂H, —(CO)NH₂, —NH(CO)H, —NR^{a}C(NH)NH₂, or —NH-dansyl group;
AA, BB, CC, DD, EE, FF, GG, and HH are each independently absent or —CH₂—;
R^{a} at each occurrence is independently a hydrogen or a substituted or unsubstituted C₁-C₄ alkyl group;
R^{b} at each occurrence is independently an ethylene-NH-dansyl or ethylene-NH-anthraniloyl group.

In some embodiments of Formula I,

A is

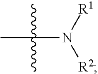

J is

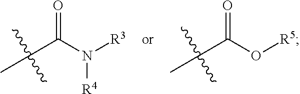

B, C, D, E, and G are each independently

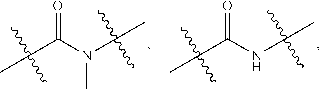

or absent;

with the proviso when f is 0, G is

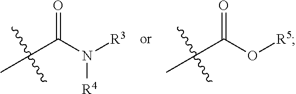

when e and f are 0, E is

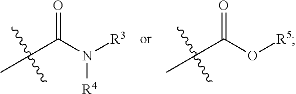

when d, e, and fare 0, D is

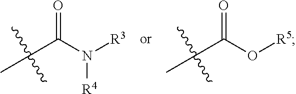

and

when c, d, e, and fare 0, C is

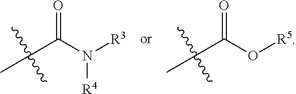

In another embodiment of Formula I,

A is

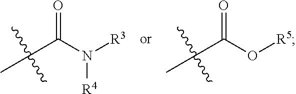

J is

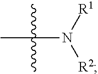

B, C, D, E, and G are each independently

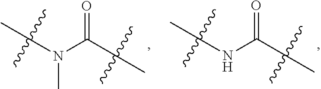

or absent;

with the proviso when f is 0, G is

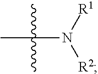

when e and f are 0, E is

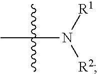

when d, e, and f are 0, D is

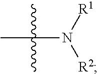

and

when c, d, e, and f are 0, C is

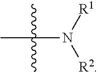

In some embodiments of Formula I, at least one of R¹⁰¹, R¹⁰², R¹⁰⁴, R¹⁰⁵, and R¹⁰⁶ is a basic group, as defined above, and at least one of R¹⁰¹, R¹⁰³, R¹⁰⁴, R¹⁰⁵, and R¹⁰⁶ is a neutral group as defined above. In some such embodiments, the neutral group is an aromatic, heterocyclic or cycloalkyl group as defined above. In some embodiments of Formula I, the peptide contains at least one arginine, such as, but not limited to D-arginine, and at least one 2′6′-dimethyltyrosine, tyrosine, or phenylalanine. In some embodiments of Formula I, R¹⁰¹ is an alkylguanidinium group.

In some embodiments, the peptide of the present technology is selected from the peptides shown in Tables A or B.

TABLE A

Tyr-D-Arg-Phe-Lys-NH₂
D-Arg-Dmt-Lys-Phe-NH₂
D-Arg-Dmt-Arg-Phe-NH₂
D-Arg-Dmt-Orn-Phe-NH₂
D-Arg-Tyr-Lys-Phe-NH₂
D-Arg-Tyr-Arg-Phe-NH₂
D-Arg-Tyr-Orn-Phe-NH₂
D-Arg-Dmt-Phe-Lys-NH₂
D-Arg-Phe-Lys-Dmt-NH₂
D-Arg-Phe-Dmt-Lys-NH₂
D-Arg-Lys-Dmt-Phe-NH₂
D-Arg-Lys-Phe-Dmt-NH₂
D-Arg-Dmt-Lys-Phe-Cys-NH₂
Phe-Lys-Dmt-D-Arg-NH₂
Phe-Lys-D-Arg-Dmt-NH₂
Phe-D-Arg-Phe-Lys-NH₂
Phe-D-Arg-Phe-Lys-Cys-NH₂
Phe-D-Arg-Phe-Lys-Ser-Cys-NH₂
Phe-D-Arg-Phe-Lys-Gly-Cys-NH₂
Phe-D-Arg-Dmt-Lys-NH₂
Phe-D-Arg-Dmt-Lys-Cys-NH₂
Phe-D-Arg-Dmt-Lys-Ser-Cys-NH₂
Phe-D-Arg-Dmt-Lys-Gly-Cys-NH₂
Phe-D-Arg-Lys-Dmt-NH₂
Phe-Dmt-D-Arg-Lys-NH₂
Phe-Dmt-Lys-D-Arg-NH₂
Lys-Phe-D-Arg-Dmt-NH₂
Lys-Phe-Dmt-D-Arg-NH₂
Lys-Dmt-D-Arg-Phe-NH₂
Lys-Dmt-Phe-D-Arg-NH₂
Lys-D-Arg-Phe-Dmt-NH₂
Lys-D-Arg-Dmt-Phe-NH₂
D-Arg-Dmt-D-Arg-Phe-NH₂
D-Arg-Dmt-D-Arg-Dmt-NH₂
D-Arg-Dmt-D-Arg-Tyr-NH₂
D-Arg-Dmt-D-Arg-Trp-NH₂
Trp-D-Arg-Tyr-Lys-NH₂
Trp-D-Arg-Trp-Lys-NH₂
Trp-D-Arg-Dmt-Lys-NH₂
D-Arg-Trp-Lys-Phe-NH₂
D-Arg-Trp-Phe-Lys-NH₂
D-Arg-Trp-Lys-Dmt-NH₂
D-Arg-Trp-Dmt-Lys-NH₂
D-Arg-Lys-Trp-Phe-NH₂
D-Arg-Lys-Trp-Dmt-NH₂
Cha-D-Arg-Phe-Lys-NH₂
Ala-D-Arg-Phe-Lys-NH₂
2′,6′-Dmp-D-Arg-2′,6′-Dmt-Lys-NH₂
2′,6′-Dmp-D-Arg-Phe-Lys-NH₂
2′,6′-Dmt-D-Arg-Phe-Orn-NH₂
2′,6′-Dmt-D-Arg-Phe-Ahp-NH₂
2′,6′-Dmt-D-Arg-Phe-Lys-NH₂
2′,6′-Dmt-D-Cit-Phe-Lys-NH₂
D-Arg-2′6′-Dmt-Lys-Phe-NH₂
D-Tyr-Trp-Lys-NH₂
Lys-D-Arg-Tyr-NH₂
Met-Tyr-D-Arg-Phe-Arg-NH₂
Met-Tyr-D-Lys-Phe-Arg
Phe-Arg-D-His-Asp
Phe-D-Arg-2′6′-Dmt-Lys-NH₂
Phe-D-Arg-His
Trp-D-Lys-Tyr-Arg-NH₂
Tyr-D-Arg-Phe-Lys-Glu-NH₂
Tyr-His-D-Gly-Met
D-Arg-Tyr-Lys-Phe-NH₂
D-Arg-D-Dmt-Lys-Phe-NH₂
D-Arg-Dmt-D-Lys-Phe-NH₂
D-Arg-Dmt-Lys-D-Phe-NH₂
D-Arg-D-Dmt-D-Lys-D-Phe-NH₂
Phe-D-Arg-D-Phe-Lys-NH₂
Phe-D-Arg-Phe-D-Lys-NH₂
D-Phe-D-Arg-D-Phe-D-Lys-NH₂
Lys-D-Phe-Arg-Dmt-NH₂
D-Arg-Arg-Dmt-Phe-NH₂
Dmt-D-Phe-Arg-Lys-NH₂
Phe-D-Dmt-Arg-Lys-NH₂
D-Arg-Dmt-Lys-NH₂
Arg-D-Dmt-Lys-NH₂
D-Arg-Dmt-Phe-NH₂
Arg-D-Dmt-Arg-NH₂
Dmt-D-Arg-NH₂
D-Arg-Dmt-NH₂
D-Dmt-Arg-NH₂
Arg-D-Dmt-NH₂
D-Arg-D-Dmt-NH₂
D-Arg-D-Tyr-Lys-Phe-NH₂
D-Arg-Tyr-D-Lys-Phe-NH₂
D-Arg-Tyr-Lys-D-Phe-NH₂
D-Arg-D-Tyr-D-Lys-D-Phe-NH₂
Lys-D-Phe-Arg-Tyr-NH₂
D-Arg-Arg-Tyr-Phe-NH₂
Tyr-D-Phe-Arg-Lys-NH₂
Phe-D-Tyr-Arg-Lys-NH₂
D-Arg-Tyr-Lys-NH₂
Arg-D-Tyr-Lys-NH₂
D-Arg-Tyr-Phe-NH₂
Arg-D-Tyr-Arg-NH₂
Tyr-D-Arg-NH₂
D-Arg-Tyr-NH₂
D-Tyr-Arg-NH₂
Arg-D-Tyr-NH₂
D-Arg-D-Tyr-NH₂
Dmt-Lys-Phe-NH₂
Lys-Dmt-D-Arg-NH₂
Phe-Lys-Dmt-NH₂
D-Arg-Phe-Lys-NH₂
D-Arg-Cha-Lys-NH₂
D-Arg-Trp-Lys-NH₂
Dmt-Lys-D-Phe-NH₂
Dmt-Lys-NH₂
Lys-Phe-NH₂
D-Arg-Cha-Lys-Cha-NH₂
D-Nle-Dmt-Ahp-Phe-NH₂
D-Nle-Cha-Ahp-Cha-NH₂
D-Arg-Dmt-D-Lys-NH₂
D-Arg-Dmt-D-Lys-Phe-NH₂
Lys-Trp-D-Arg-NH₂
H-Lys-D-Phe-Arg-Dmt-NH₂
H-D-Arg-Lys-Dmt-Phe-NH₂
H-D-Arg-Lys-Phe-Dmt-NH₂
H-D-Arg-Arg-Dmt-Phe-NH₂
H-D-Arg-Dmt-Phe-Lys-NH₂
H-D-Arg-Phe-Dmt-Lys-NH₂
H-Dmt-D-Phe-Arg-Lys-NH₂
H-Phe-D-Dmt-Arg-Lys-NH₂
H-D-Arg-Dmt-Lys-NH₂
H-D-Arg-Dmt-D-Lys-D-Phe-NH₂
H-D-Arg-D-Dmt-Lys-Phe-NH₂
H-D-Arg-Dmt-Phe-NH₂
H-Dmt-D-Arg-NH₂
H-Phe-D-Arg-D-Phe-Lys-NH₂
H-Phe-D-Arg-Phe-D-Lys-NH₂
H-D-Phe-D-Arg-D-Phe-D-Lys-NH₂
H-D-Arg-D-Dmt-D-Lys-D-Phe-NH₂
H-D-Arg-Cha-Lys-NH₂
H-D-Arg-Cha-Lys-Cha-NH₂
H-Arg-D-Dmt-Lys-NH₂
H-Arg-D-Dmt-Arg-NH₂
H-D-Dmt-Arg-NH₂
H-Arg-D-Dmt-NH₂
H-D-Arg-D-Dmt-NH₂
Arg-Arg-Dmt-Phe
Arg-Cha-Lys
Arg-Dmt
Arg-Dmt-Arg
Arg-Dmt-Lys
Arg-Dmt-Lys-Phe
Arg-Dmt-Lys-Phe-Cys
Arg-Dmt-Phe
Arg-Dmt-Phe-Lys
Arg-Lys-Dmt-Phe
Arg-Lys-Phe-Dmt
Arg-Phe-Dmt-Lys
Arg-Phe-Lys
Arg-Trp-Lys
Arg-Tyr-Lys
Arg-Tyr-Lys-Phe
D-Arg-D-Dmt-D-Lys-L-Phe-NH₂
D-Arg-D-Dmt-L-Lys-D-Phe-NH₂
D-Arg-D-Dmt-L-Lys-L-Phe-NH₂
D-Arg-Dmt-D-Lys-NH₂
D-Arg-Dmt-Lys-NH₂
D-Arg-Dmt-Lys-Phe-Cys
D-Arg-L-Dmt-D-Lys-D-Phe-NH₂
D-Arg-L-Dmt-D-Lys-L-Phe-NH₂
D-Arg-L-Dmt-L-Lys-D-Phe-NH₂
Dmt-Arg
Dmt-Lys
Dmt-Lys-Phe
Dmt-Phe-Arg-Lys
H-Arg-D-Dmt-Lys-Phe-NH₂
H-Arg-Dmt-Lys-Phe-NH₂
H-D-Arg-2,6-dichloro-L-tyrosine-L-Lys-L-Phe-NH₂
H-D-Arg-2,6-dichlorotyrosine-Lys-Phe-NH₂
H-D-Arg-2,6-difluoro-L-tyrosine-L-Lys-L-Phe-NH₂
H-D-Arg-2,6-difluorotyrosine-Lys-Phe-NH₂
H-D-Arg-2,6-dimethyl-L-phenylalanine-L-Lys-L-Phe-
NH₂
H-D-Arg-2,6-dimethylphenylalanine-Lys-Phe-NH₂
H-D-Arg-4-methoxy-2,6-dimethyl-L-phenylalanine-L-
Lys-L-Phe-NH₂
H-D-Arg-4-methoxy-2,6-dimethylphenylalanine-Lys-
Phe-NH₂
H-D-Arg-Dmt-Lys-2,6-dimethylphenylalanine-NH₂
H-D-Arg-Dmt-Lys-3-hydroxyphenylalanine-NH₂
H-D-Arg-Dmt-N6-acetyllysine-Phe-NH₂
H-D-Arg-D-Phe-L-Lys-L-Phe-NH₂
H-D-Arg-D-Trp-L-Lys-L-Phe-NH₂
H-D-Arg-D-Tyr-L-Lys-L-Phe-NH₂
H-D-Arg-L-Dmt-L-Lys-2,6-dimethyl-L-phenylalanine-
NH₂
H-D-Arg-L-Dmt-L-Lys-3-hydroxy-L-phenylalanine-NH₂
H-D-Arg-L-Dmt-L-Lys-D-Dmt-NH₂
H-D-Arg-L-Dmt-L-Lys-D-Trp-NH₂
H-D-Arg-L-Dmt-L-Lys-D-Tyr-NH₂
H-D-Arg-L-Dmt-L-Lys-L-Dmt-NH₂
H-D-Arg-L-Dmt-L-Lys-L-Trp-NH₂
H-D-Arg-L-Dmt-L-Lys-L-Tyr-NH₂
H-D-Arg-L-Dmt-L-Phe-L-Lys-NH₂
H-D-Arg-L-Dmt-N6-acetyl-L-lysine-L-Phe-NH₂
H-D-Arg-L-Lys-L-Dmt-L-Phe-NH₂
H-D-Arg-L-Lys-L-Phe-L-Dmt-NH₂
H-D-Arg-L-Phe-L-Dmt-L-Lys-NH₂
H-D-Arg-L-Phe-L-Lys-L-Dmt-NH₂
H-D-Arg-L-Phe-L-Lys-L-Phe-NH₂
H-D-Arg-L-Trp-L-Lys-L-Phe-NH₂
H-D-Arg-L-Tyr-L-Lys-L-Phe-NH₂
H-D-Arg-Phe-Lys-Dmt-NH₂
H-D-Arg-Tyr-Lys-Phe-NH₂
H-D-His-L-Dmt-L-Lys-L-Phe-NH₂
H-D-Lys-L-Dmt-L-Lys-L-Phe-NH₂
H-Dmt-D-Arg-Lys-Phe-NH₂
H-Dmt-D-Arg-Phe-Lys-NH₂
H-Dmt-Lys-D-Arg-Phe-NH₂
H-Dmt-Lys-Phe-D-Arg-NH₂
H-Dmt-Phe-D-Arg-Lys-NH₂
H-Dmt-Phe-Lys-D-Arg-NH₂
H-L-Dmt-D-Arg-L-Lys-L-Phe-NH₂
H-L-Dmt-D-Arg-L-Phe-L-Lys-NH₂
H-L-Dmt-L-Lys-D-Arg-L-Phe-NH₂
H-L-Dmt-L-Lys-L-Phe-D-Arg-NH₂
H-L-Dmt-L-Phe-D-Arg-L-Lys-NH₂
H-L-Dmt-L-Phe-L-Lys-D-Arg-NH₂
H-L-His-L-Dmt-L-Lys-L-Phe-NH₂
H-L-Lys-D-Arg-L-Dmt-L-Phe-NH₂
H-L-Lys-D-Arg-L-Phe-L-Dmt-NH₂
H-L-Lys-L-Dmt-D-Arg-L-Phe-NH₂
H-L-Lys-L-Dmt-L-Lys-L-Phe-NH₂
H-L-Lys-L-Dmt-L-Phe-D-Arg-NH₂
H-L-Lys-L-Phe-D-Arg-L-Dmt-NH₂
H-L-Lys-L-Phe-L-Dmt-D-Arg-NH₂
H-L-Phe-D-Arg-L-Dmt-L-Lys-NH₂
H-L-Phe-D-Arg-L-Lys-L-Dmt-NH₂
H-L-Phe-L-Dmt-D-Arg-L-Lys-NH₂
H-L-Phe-L-Dmt-L-Lys-D-Arg-NH₂
H-L-Phe-L-Lys-D-Arg-L-Dmt-NH₂
H-L-Phe-L-Lys-L-Dmt-D-Arg-NH₂
H-Lys-D-Arg-Dmt-Phe-NH₂
H-Lys-D-Arg-Phe-Dmt-NH₂
H-Lys-Dmt-D-Arg-Phe-NH₂
H-Lys-Dmt-Phe-D-Arg-NH₂
H-Lys-Phe-D-Arg-Dmt-NH₂
H-Lys-Phe-Dmt-D-Arg-NH₂
H-Phe-Arg-Phe-Lys-NH₂
H-Phe-D-Arg-Dmt-Lys-NH₂
H-Phe-D-Arg-Lys-Dmt-NH₂
H-Phe-Dmt-D-Arg-Lys-NH₂
H-Phe-Dmt-Lys-D-Arg-NH₂
H-Phe-Lys-D-Arg-Dmt-NH₂
H-Phe-Lys-Dmt-D-Arg-NH₂
L-Arg-D-Dmt-D-Lys-D-Phe-NH₂
L-Arg-D-Dmt-D-Lys-L-Phe-NH₂
L-Arg-D-Dmt-L-Lys-D-Phe-NH₂
L-Arg-D-Dmt-L-Lys-L-Phe-NH₂
L-Arg-L-Dmt-D-Lys-D-Phe-NH₂
L-Arg-L-Dmt-D-Lys-L-Phe-NH₂
L-Arg-L-Dmt-L-Lys-D-Phe-NH₂
L-Arg-L-Dmt-L-Lys-L-Phe-NH₂
Lys-Dmt-Arg
Lys-Phe
Lys-Phe-Arg-Dmt
Lys-Trp-Arg
Phe-Arg-Dmt-Lys
Phe-Arg-Phe-Lys
Phe-Dmt-Arg-Lys
Phe-Lys-Dmt
Arg-Dmt-Lys-Phe-NH₂
Phe-Dmt-Arg-Lys-NH₂
Phe-Lys-Dmt-Arg-NH₂
Dmt-Arg-Lys-Phe-NH₂
Lys-Dmt-Arg-Phe-NH₂
Phe-Dmt-Lys-Arg-NH₂
Arg-Lys-Dmt-Phe-NH₂
Arg-Dmt-Phe-Lys-NH₂
D-Arg-Dmt-Lys-Phe-NH₂
Dmt-D-Arg-Phe-Lys-NH₂
H-Phe-D-Arg Phe-Lys-Cys-NH₂
D-Arg-Dmt-Lys-Trp-NH₂
D-Arg-Trp-Lys-Trp-NH₂
H-D-Arg-Dmt-Lys-Phe(NMe)-NH₂
H-D-Arg-Dmt-Lys(N^{α}Me)-Phe(NMe)-NH₂
H-D-Arg(N^{α}Me)-Dmt(NMe)-Lys(N^{α}Me)-Phe(NMe)-NH₂
D-Arg-2′6′Dmt-Lys-Phe-NH₂
H-Phe-D-Arg-Phe-Lys-Cys-NH₂
D-Arg-Dmt-Lys-Phe-Ser-Cys-NH₂
D-Arg-Dmt-Lys-Phe-Gly-Cys-NH₂
Gly-D-Phe-Lys-His-D-Arg-Tyr-NH₂
D-Arg-Dmt-Lys-Phe-Met-NH₂
D-Arg-Dmt-Lys-Phe-Lys-Trp-NH₂
D-Arg-Dmt-Lys-Dmt-Lys-Trp-NH₂
D-Arg-Dmt-Lys-Phe-Lys-Met-NH₂
D-Arg-Dmt-Lys-Dmt-Lys-Met-NH₂
H-D-Arg-Dmt-Lys-OH
H-D-Arg-Dmt-OH
H-D-Arg-Dmt-Lys-Phe-OH

TABLE B

| Amino Acid | Amino Acid | Amino Acid | Amino Acid | C-Terminal |
|---|---|---|---|---|
| Position 1 | Position 2 | Position 3 | Position 4 | Modification |
| Tyr | D-Arg | Phe | Orn | NH₂ |
| Tyr | D-Arg | Phe | Dab | NH₂ |
| Tyr | D-Arg | Phe | Dap | NH₂ |
| 2′6′Dmt | D-Arg | Phe | Lys-NH(CH₂)₂-NH-dns | NH₂ |
| 2′6′Dmt | D-Arg | Phe | Lys-NH(CH₂)₂-NH-atn | NH₂ |
| 2′6′Dmt | D-Arg | Phe | dnsLys | NH₂ |
| 2′6′Dmt | D-Cit | Phe | Ahp | NH₂ |
| 2′6′Dmt | D-Arg | Phe | Dab | NH₂ |
| 2′6′Dmt | D-Arg | Phe | Dap | NH₂ |
| 3′5′Dmt | D-Arg | Phe | Lys | NH₂ |
| 3′5′Dmt | D-Arg | Phe | Orn | NH₂ |
| 3′5′Dmt | D-Arg | Phe | Dab | NH₂ |
| 3′5′Dmt | D-Arg | Phe | Dap | NH₂ |
| Tyr | D-Arg | Tyr | Lys | NH₂ |
| Tyr | D-Arg | Tyr | Orn | NH₂ |
| Tyr | D-Arg | Tyr | Dab | NH₂ |
| Tyr | D-Arg | Tyr | Dap | NH₂ |
| 2′6′Dmt | D-Arg | Tyr | Lys | NH₂ |
| 2′6′Dmt | D-Arg | Tyr | Orn | NH₂ |
| 2′6′Dmt | D-Arg | Tyr | Dab | NH₂ |
| 2′6′Dmt | D-Arg | Tyr | Dap | NH₂ |
| 2′6′Dmt | D-Arg | 2′6′Dmt | Lys | NH₂ |
| 2′6′Dmt | D-Arg | 2′6′Dmt | Orn | NH₂ |
| 2′6′Dmt | D-Arg | 2′6′Dmt | Dab | NH₂ |
| 2′6′Dmt | D-Arg | 2′6′Dmt | Dap | NH₂ |
| 3′5′Dmt | D-Arg | 3′5′Dmt | Arg | NH₂ |
| 3′5′Dmt | D-Arg | 3′5′Dmt | Lys | NH₂ |
| 3′5′Dmt | D-Arg | 3′5′Dmt | Orn | NH₂ |
| 3′5′Dmt | D-Arg | 3′5′Dmt | Dab | NH₂ |
| Tyr | D-Lys | Phe | Dap | NH₂ |
| Tyr | D-Lys | Phe | Arg | NH₂ |
| Tyr | D-Lys | Phe | Lys | NH₂ |
| Tyr | D-Lys | Phe | Orn | NH₂ |
| 2′6′Dmt | D-Lys | Phe | Dab | NH₂ |
| 2′6′Dmt | D-Lys | Phe | Dap | NH₂ |
| 2′6′Dmt | D-Lys | Phe | Arg | NH₂ |
| 2′6′Dmt | D-Lys | Phe | Lys | NH₂ |
| 3′5′Dmt | D-Lys | Phe | Orn | NH₂ |
| 3′5′Dmt | D-Lys | Phe | Dab | NH₂ |
| 3′5′Dmt | D-Lys | Phe | Dap | NH₂ |
| 3′5′Dmt | D-Lys | Phe | Arg | NH₂ |
| Tyr | D-Lys | Tyr | Lys | NH₂ |
| Tyr | D-Lys | Tyr | Orn | NH₂ |
| Tyr | D-Lys | Tyr | Dab | NH₂ |
| Tyr | D-Lys | Tyr | Dap | NH₂ |
| 2′6′Dmt | D-Lys | Tyr | Lys | NH₂ |
| 2′6′Dmt | D-Lys | Tyr | Orn | NH₂ |
| 2′6′Dmt | D-Lys | Tyr | Dab | NH₂ |
| 2′6′Dmt | D-Lys | Tyr | Dap | NH₂ |
| 2′6′Dmt | D-Lys | 2′6′Dmt | Lys | NH₂ |
| 2′6′Dmt | D-Lys | 2′6′Dmt | Orn | NH₂ |
| 2′6′Dmt | D-Lys | 2′6′Dmt | Dab | NH₂ |
| 2′6′Dmt | D-Lys | 2′6′Dmt | Dap | NH₂ |
| 3′5′Dmt | D-Lys | 3′5′Dmt | Lys | NH₂ |
| 3′5′Dmt | D-Lys | 3′5′Dmt | Orn | NH₂ |
| 3′5′Dmt | D-Lys | 3′5′Dmt | Dab | NH₂ |
| 3′5′Dmt | D-Lys | 3′5′Dmt | Dap | NH₂ |
| Tyr | D-Lys | Phe | Arg | NH₂ |
| Tyr | D-Orn | Phe | Arg | NH₂ |
| Tyr | D-Dab | Phe | Arg | NH₂ |
| Tyr | D-Dap | Phe | Arg | NH₂ |
| 2′6′Dmt | D-Arg | Phe | Arg | NH₂ |
| 2′6′Dmt | D-Lys | Phe | Arg | NH₂ |
| 2′6′Dmt | D-Orn | Phe | Arg | NH₂ |
| 2′6′Dmt | D-Dab | Phe | Arg | NH₂ |
| 3′5′Dmt | D-Dap | Phe | Arg | NH₂ |
| 3′5′Dmt | D-Arg | Phe | Arg | NH₂ |
| 3′5′Dmt | D-Lys | Phe | Arg | NH₂ |
| 3′5′Dmt | D-Orn | Phe | Arg | NH₂ |
| Tyr | D-Lys | Tyr | Arg | NH₂ |
| Tyr | D-Orn | Tyr | Arg | NH₂ |
| Tyr | D-Dab | Tyr | Arg | NH₂ |
| Tyr | D-Dap | Tyr | Arg | NH₂ |
| 2′6′Dmt | D-Arg | 2′6′Dmt | Arg | NH₂ |
| 2′6′Dmt | D-Lys | 2′6′Dmt | Arg | NH₂ |
| 2′6′Dmt | D-Orn | 2′6′Dmt | Arg | NH₂ |
| 2′6′Dmt | D-Dab | 2′6′Dmt | Arg | NH₂ |
| 3′5′Dmt | D-Dap | 3′5′Dmt | Arg | NH₂ |
| 3′5′Dmt | D-Arg | 3′5′Dmt | Arg | NH₂ |
| 3′5′Dmt | D-Lys | 3′5′Dmt | Arg | NH₂ |
| 3′5′Dmt | D-Orn | 3′5′Dmt | Arg | NH₂ |
| Mmt | D-Arg | Phe | Lys | NH₂ |
| Mmt | D-Arg | Phe | Orn | NH₂ |
| Mmt | D-Arg | Phe | Dab | NH₂ |
| Mmt | D-Arg | Phe | Dap | NH₂ |
| Tmt | D-Arg | Phe | Lys | NH₂ |
| Tmt | D-Arg | Phe | Orn | NH₂ |
| Tmt | D-Arg | Phe | Dab | NH₂ |
| Tmt | D-Arg | Phe | Dap | NH₂ |
| Hmt | D-Arg | Phe | Lys | NH₂ |
| Hmt | D-Arg | Phe | Orn | NH₂ |
| Hmt | D-Arg | Phe | Dab | NH₂ |
| Hmt | D-Arg | Phe | Dap | NH₂ |
| Mmt | D-Lys | Phe | Lys | NH₂ |
| Mmt | D-Lys | Phe | Orn | NH₂ |
| Mmt | D-Lys | Phe | Dab | NH₂ |
| Mmt | D-Lys | Phe | Dap | NH₂ |
| Mmt | D-Lys | Phe | Arg | NH₂ |
| Tmt | D-Lys | Phe | Lys | NH₂ |
| Tmt | D-Lys | Phe | Orn | NH₂ |
| Tmt | D-Lys | Phe | Dab | NH₂ |
| Tmt | D-Lys | Phe | Dap | NH₂ |
| Tmt | D-Lys | Phe | Arg | NH₂ |
| Hmt | D-Lys | Phe | Lys | NH₂ |
| Hmt | D-Lys | Phe | Orn | NH₂ |
| Hmt | D-Lys | Phe | Dab | NH₂ |
| Hmt | D-Lys | Phe | Dap | NH₂ |
| Hmt | D-Lys | Phe | Arg | NH₂ |
| Mmt | D-Lys | Phe | Arg | NH₂ |
| Mmt | D-Orn | Phe | Arg | NH₂ |
| Mmt | D-Dab | Phe | Arg | NH₂ |
| Mmt | D-Dap | Phe | Arg | NH₂ |
| Mmt | D-Arg | Phe | Arg | NH₂ |
| Tmt | D-Lys | Phe | Arg | NH₂ |
| Tmt | D-Orn | Phe | Arg | NH₂ |
| Tmt | D-Dab | Phe | Arg | NH₂ |
| Tmt | D-Dap | Phe | Arg | NH₂ |
| Tmt | D-Arg | Phe | Arg | NH₂ |
| Hmt | D-Lys | Phe | Arg | NH₂ |
| Hmt | D-Orn | Phe | Arg | NH₂ |
| Hmt | D-Dab | Phe | Arg | NH₂ |
| Hmt | D-Dap | Phe | Arg | NH₂ |
| Hmt | D-Arg | Phe | Arg | NH₂ |
| Trp | D-Arg | Phe | Lys | NH₂ |

2′-methyltyrosine (Mmt); Dimethyltyrosine (Dmt); 2′,6′-dimethyltyrosine (2′6′-Dmt); 3′,5′-dimethyltyrosine (3′5′Dmt); N,2′,6′-trimethyltyrosine (Tmt); 2′-hydroxy-6′-methyltyrosine (Hmt); 2′-methylphenylalanine (Mmp); dimethylphenylalanine (Dmp) 2′,6′-dimethylphenylalanine (2′,6′-Dmp); N,2′,6′-trimethylphenylalanine (Tmp); 2′ -hydroxy-6′-methylphenylalanine (Hmp); cyclohexylalanine (Cha); diaminobutyric (Dab); diaminopropionic acid (Dap); β-dansyl-L-α,β-diaminopropionic acid (dnsDap); β-anthraniloyl-L-α,β-diaminopropionic acid (atnDap); biotin (bio); norleucine (Nle); 2-aminohepantoic acid (Ahp); β-(6′-dimethylamino-2′-naphthoyl)alanine (Ald); Sarcosine (Sar); Ornithine (Orn)

In another embodiment, the peptide is defined by Formula II:

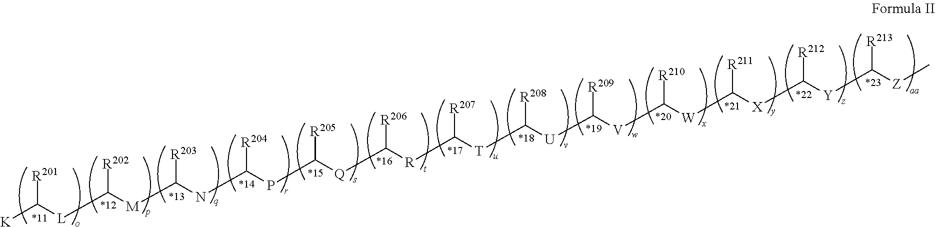

wherein:

one of K and Z is

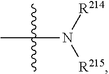

and the other of K and Z is

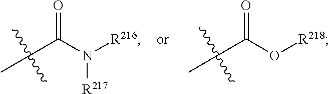

L, M, N, P, Q, R, T, U, V, W, X, and Y are each

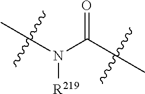

or L, M, N, P, Q, R, T, U, V, W, X, and Y are each

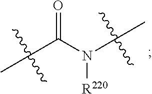

with the proviso that when
aa is 0 and Z is not a terminal group, the terminal group is one of L, M, P, Q, R, T, U, V, W, X, or Y, such that one of K and the terminal group is

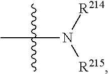

and the other of K and the terminal group is selected from

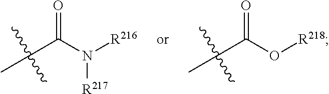

R²⁰¹ is

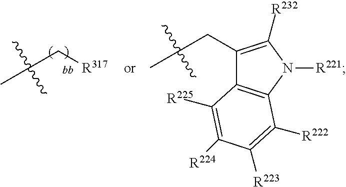

R²⁰² is

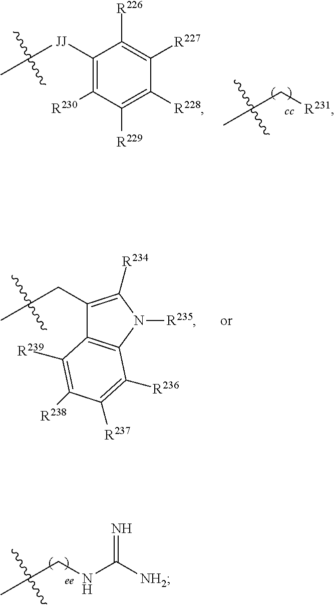

R²⁰³ is

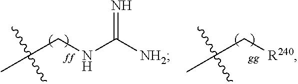

or hydrogen;

R²⁰⁴ is

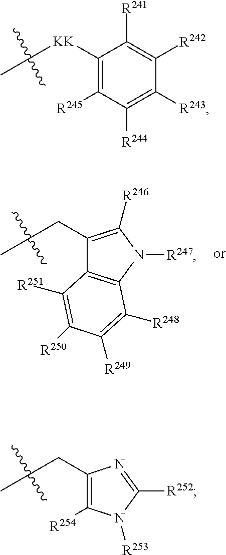

R²⁰⁵ is

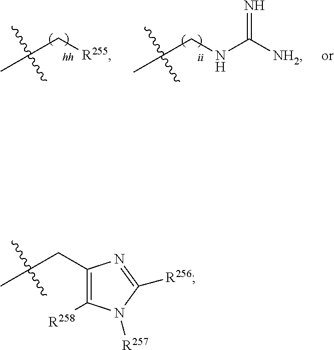

R²⁰⁶ is

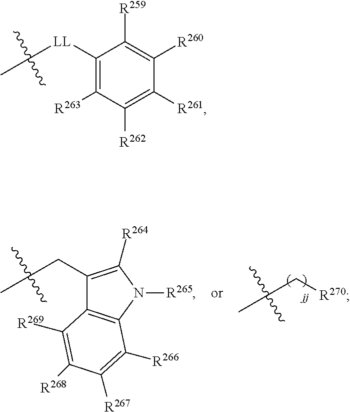

R²⁰⁷ is

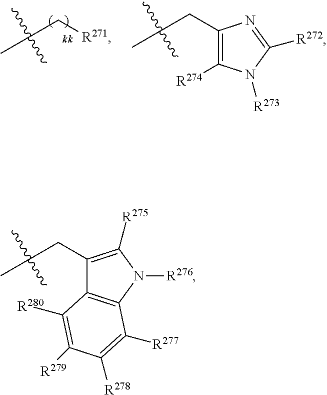

or hydrogen;

R²⁰⁸ is

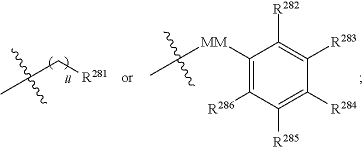

R²⁰⁹ is

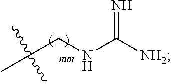

R²¹⁰ is

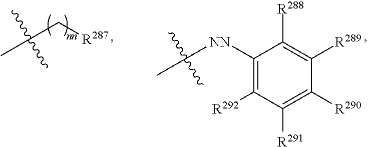

or hydrogen;

R²¹¹ is

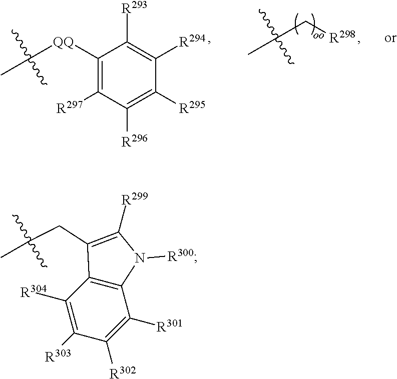

R²¹² is

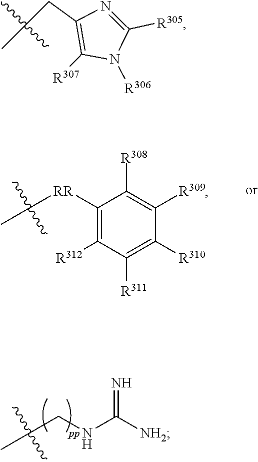

R²¹³ is

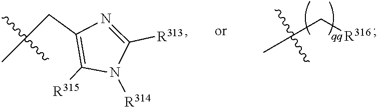

wherein
[symbol] , [symbol] , [symbol] , [symbol] , [symbol] , [symbol] , [symbol] , [symbol] , [symbol] , and [symbol] each independently indicate carbon stereocenters, an absolute configuration of each of which is independently at each occurrence R or S;
when R²⁰³ is not hydrogen, then [symbol] indicates a carbon stereocenter with an absolute configuration of R or S;
when R²⁰⁷ is not hydrogen, then [symbol] indicates a carbon stereocenter with an absolute configuration of R or S;
when R²¹⁰ is not hydrogen, then [symbol] indicates a carbon stereocenter with an absolute configuration of R or S;
R²¹⁴, R²¹⁵, R²¹⁶, R²¹⁷, and R²¹⁸ are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, heteroaryl, or amino protecting group; or R²¹⁴ and R²¹⁵ together form a 3, 4, 5, 6, 7, or 8 membered substituted or unsubstituted heterocycyl ring;
R²¹⁹ and R²²⁰ are, at each occurrence, independently a hydrogen or substituted or unsubstituted C₁-C₆ alkyl group;
R²²², R²²³, R²²⁴, R²²⁵, R²²⁶, R²²⁷, R²²⁸, R²²⁹, R²³⁰, R²³², R²³⁴, R²³⁶, R²³⁷, R²³⁸, R²³⁹, R²⁴¹, R²⁴², R²⁴³, R²⁴⁴, R²⁴⁵, R²⁴⁶, R²⁴⁸, R²⁴⁹, R²⁵⁰, R²⁵¹, R²⁵², R²⁵⁴, R²⁵⁶, R²⁵⁸, R²⁵⁹, R²⁶⁰, R²⁶¹, R²⁶², R²⁶³, R²⁶⁴, R²⁶⁶, R²⁶⁷, R²⁶⁸, R²⁶⁹, R²⁷², R²⁷⁴, R²⁷⁵, R²⁷⁷, R²⁷⁸, R²⁷⁹, R²⁸⁰, R²⁸², R²⁸³, R²⁸⁴, R²⁸⁵, R²⁸⁶, R²⁸⁸, R²⁸⁹, R²⁹⁰, R²⁹¹, R²⁹², R²⁹³, R²⁹⁴, R²⁹⁵, R²⁹⁶, R²⁹⁷, R²⁹⁹, R³⁰¹, R³⁰², R³⁰³, R³⁰⁴, R³⁰⁵, R³⁰⁷, R³⁰⁸, R³⁰⁹, R³¹⁰, R³¹¹, R³¹², R³¹³, and R³¹⁵ are each independently a hydrogen, amino, amido, —NO₂, —CN, —OR^{c}, —SR^{c}, —NR^{c}R^{c}, —F, —Cl, —Br, —I, or a substituted or unsubstituted C₁-C₆ alkyl, C₁-C₆ alkoxy, —C(O)-alkyl, —C(O)-aryl, —C(O)— aralkyl, —C(O)₂R^{c}, C₁-C₄ alkylamino, C₁-C₄ dialkylamino, or perhaloalkyl group;
R²²¹, R²³⁵, R²⁴⁷, R²⁵³, R²⁵⁷, R²⁶⁵, R²⁷³, R²⁷⁶, R³⁰⁰, R³⁰⁶, and R³¹⁴ are each independently a hydrogen or substituted or unsubstituted C₁-C₆ alkyl group;

R²³¹, R²⁴⁰, R²⁵⁵, R²⁷⁰, R²⁷¹, R²⁸¹, R²⁸⁷, R²⁹⁸, R³¹⁶, and R³¹⁷ are each independently a hydrogen, —OR^{c}, —SR^{c}, —NR^{c}R^{c}; —NR^{c}R^{d}, —CO₂R^{c}, —(CO)NR^{c}R^{c}, —NR^{c}(CO)R^{c}, —NR^{c}C(NH)NH₂, —NR^{c}-dansyl, or a substituted or unsubstituted alkyl, aryl, or aralkyl group;
JJ, KK, LL, MM, NN, QQ, and RR are each independently absent, —NH(CO)—, or —CH₂—;
R^{c} at each occurrence is independently a hydrogen or a substituted or unsubstituted C₁-C₆ alkyl group;
R^{d} at each occurrence is independently a C₁-C₆ alkylene-NR^{c}-dansyl or C₁-C₆ alkylene-NR^{c}-anthraniloyl group;
o, p, q, r, s, t, u, v, w, x, y, z, and aa are each independently 0 or 1, with the proviso that o+p+q+r+s+t+u+v+w+x+y+z+aa equals 6, 7, 8, 9, 10, or 11;
cc is 0, 1, 2, 3, 4, or 5; and
bb, cc, ee, ff, gg, hh, ii, jj, kk, ll, mm, nn, oo, pp, and qq are each independently 1, 2, 3, 4, or 5.

In some embodiments of peptides of Formula II,
R²¹⁴, R²¹⁵, R²¹⁶, R²¹⁷, and R²¹⁸ are each independently a hydrogen or substituted or unsubstituted C₁-C₆ alkyl group;
R²¹⁹ and R²²⁰ are, at each occurrence, independently a hydrogen or methyl group;
R²²², R²²³, R²²⁴, R²²⁵, R²²⁶, R²²⁷, R²²⁸, R²²⁹, R²³⁰, R²³², R²³⁴, R²³⁶, R²³⁷, R²³⁸, R²³⁹, R²⁴¹, R²⁴², R²⁴³, R²⁴⁴, R²⁴⁵, R²⁴⁶, R²⁴⁸, R²⁴⁹, R²⁵⁰, R²⁵¹, R²⁵², R²⁵⁴, R²⁵⁶, R²⁵⁸, R²⁵⁹, R²⁶⁰, R²⁶¹, R²⁶², R²⁶³, R²⁶⁴, R²⁶⁶, R²⁶⁷, R²⁶⁸, R²⁶⁹, R²⁷², R²⁷⁴, R²⁷⁵, R²⁷⁷, R²⁷⁸, R²⁷⁹, R²⁸⁰, R²⁸², R²⁸³, R²⁸⁴, R²⁸⁵, R²⁸⁶, R²⁸⁸, R²⁸⁹, R²⁹⁰, R²⁹¹, R²⁹², R²⁹³, R²⁹⁴, R²⁹⁵, R²⁹⁶, R²⁹⁷, R²⁹⁹, R³⁰¹, R³⁰², R³⁰³, R³⁰⁴, R³⁰⁵, R³⁰⁷, R³⁰⁸, R³⁰⁹, R³¹⁰, R³¹¹, R³¹², R³¹³, and R³¹⁵ are each independently a hydrogen, methyl, or —OR^{c} group;
R²²¹, R²³⁵, R²⁴⁷, R²⁵³, R²⁵⁷, R²⁶⁵, R²⁷³, R²⁷⁶, R³⁰⁰, R³⁰⁶, and R³¹⁴ are each independently a hydrogen or substituted or unsubstituted C₁-C₆ alkyl group;
R²³¹ is —(CO)NR^{c}R^{c}, —OR′, or a C₁-C₆ alkyl group, optionally substituted with a hydroxyl or methyl group;
R²⁴⁰ and R²⁵⁵ are each independently —CO₂R^{c} or —NR^{c}R^{c};
R²⁷⁰ and R²⁷¹ are each independently —CO₂R^{c};
R²⁸¹ is —SR^{c} or —NR^{c}R^{c};
R²⁸⁷ —(CO)NR^{c}R^{c} or —OR^{c};
R²⁹⁸ —NR^{c}R^{c}, —CO₂R^{c}, or —SR^{c};
R³¹⁶ is —NR^{c}R^{c};
R³¹⁷ is hydrogen or —NR^{c}R^{c};
JJ, KK, LL, MM, NN, QQ, and RR are each independently absent or —CH₂—;
R^{c} at each occurrence is independently a hydrogen or a substituted or unsubstituted C₁-C₆ alkyl group;
R^{d} at each occurrence is independently a C₁-C₆ alkylene-NR^{c}-dansyl or C₁-C₆ alkylene-NR^{c}-anthraniloyl group;
o, p, q, r, s, t, u, v, w, x, y, z, and aa are each independently 0 or 1, with the proviso that o+p+q+r+s+t+u+v+w+x+y+z+aa equals 6, 7, 8, 9, 10, or 11;
cc is 0, 1, 2, 3, 4, or 5; and
bb, cc, dd, ee, ff, gg, hh, ii, jj, kk, ll, mm, nn, oo, pp, and qq are each independently 1, 2, 3, 4, or 5.

In some embodiments of peptides of Formula II,
R²²¹, R²²², R²²³, R²²⁴, R²²⁵, R²²⁶, R²²⁷, R²²⁸, R²²⁹, R²³⁰, R²³², R²³⁴, R²³⁵, R²³⁶, R²³⁷, R²³⁸, R²³⁹, R²⁴², R²⁴⁴, R²⁴⁶, R²⁴⁷, R²⁴⁸, R²⁴⁹, R²⁵⁰, R²⁵¹, R²⁵², R²⁵³, R²⁵⁴, R²⁵⁶, R²⁵⁷, R²⁵⁸, R²⁵⁹, R²⁶⁰, R²⁶², R²⁶³, R²⁶⁴, R²⁶⁵, R²⁶⁶, R²⁶⁷, R²⁶⁸, R²⁶⁹, R²⁷², R²⁷³, R²⁷⁴, R²⁷⁵, R²⁷⁶, R²⁷⁷, R²⁷⁸, R²⁷⁹, R²⁸⁰, R²⁸², R²⁸³, R²⁸⁵, R²⁸⁶, R²⁸⁸, R²⁸⁹, R²⁹¹, R²⁹², R²⁹³, R²⁹⁴, R²⁹⁶, R²⁹⁷, R²⁹⁹, R³⁰⁰, R³⁰¹, R³⁰², R³⁰³, R³⁰⁴, R³⁰⁵, R³⁰⁶, R³⁰⁷, R³⁰⁸, R³⁰⁹, R³¹¹, R³¹², R³¹³, R³¹⁴, and R³¹⁵ are each hydrogen;
R²⁴¹ and R²⁴⁵ are each independently a hydrogen or methyl group;
R²⁴³, R²⁶¹, R²⁸⁴, R²⁹⁰, R²⁹⁵, R³¹⁰ are each independently a hydrogen or OH;
R²³¹ is —(CO)NH₂, an ethyl group substituted with a hydroxyl group, or an isopropyl group;
R²⁴⁰ and R²⁵⁵ are each independently —CO₂H or —NH₂;
R²⁷⁰ and R²⁷¹ are each independently —CO₂H;
R²⁸¹ is —SH or —NH₂;
R²⁸⁷ is —(CO)NH₂ or —OH;
R²⁹⁸ is —NH₂, —CO₂H, or —SH;
R₃₁₆ is —NH₂;
R³¹⁷ is hydrogen or —NH₂;
JJ, KK, LL, MM, NN, QQ, and RR are each independently —CH₂—;
o, p, q, r, s, t, u, v, w, x, y, z, and aa are each independently 0 or 1, with the proviso that o+p+q+r+s+t+u+v+w+x+y+z+aa equals 6, 7, 8, 9, 10, or 11;
cc is 0, 1, 2, 3, 4, or 5; and
bb, cc, dd, ee, ff, gg, hh, ii, jj, kk, ll, mm, nn, oo, pp, and qq are each independently 1, 2, 3, 4, or 5.

In certain embodiments of Formula II,

K is

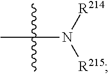

Z is

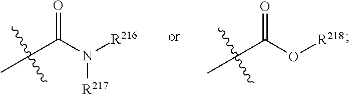

L, M, N, P, Q, R, T, U, V, W, X, and Y are each independently

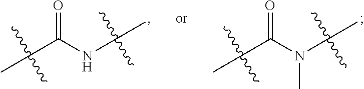

with the proviso that when
   aa is 0 and Z is not a terminal group, the terminal group is one of L, M, N, P, Q, R, T, U, V, W, X, or Y, such that one of L, M, N, P, Q, R, T, U, V, W, X, or Y, is

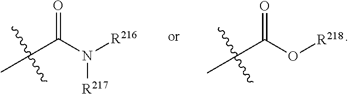

In another embodiment of Formula II,

K is

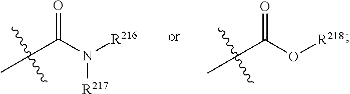

Z is

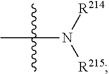

L, M, N, P, Q, R, T, U, V, W, X, and Y are each independently

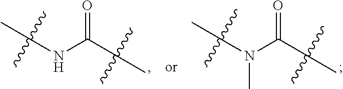

with the proviso that when
   aa is 0 and Z is not a terminal group, the terminal group is one of L, M, N, P, Q, R, T, U, V, W, X, or Y, such that one of L, M, N, P, Q, R, T, U, V, W, X, or Y, is

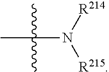

In some embodiments, the peptide of Formula II is selected from the peptides shown in Table C.

TABLE C

D-Arg-Dmt-Lys-Phe-Glu-Cys-Gly-NH₂
Phe-D-Arg-Phe-Lys-Glu-Cys-Gly-NH₂
Phe-D-Arg-Dmt-Lys-Glu-Cys-Gly-NH₂
Ala-D-Phe-D-Arg-Tyr-Lys-D-Trp-His-D-Tyr-Gly-Phe
Asp-D-Trp-Lys-Tyr-D-His-Phe-Arg-D-Gly-Lys-NH₂
D-His-Glu-Lys-Tyr-D-Phe-Arg
D-His-Lys-Tyr-D-Phe-Glu-D-Asp-D-Asp-D-His-D-Lys-
Arg-Trp-NH₂
Lys-D-G1n-Tyr-Arg-D-Phe-Trp-NH₂
Lys-Trp-D-Tyr-Arg-Asn-Phe-Tyr-D-His-NH₂
Phe-D-Arg-Lys-Trp-Tyr-D-Arg-His
Thr-Gly-Tyr-Arg-D-His-Phe-Trp-D-His-Lys
Trp-Lys-Phe-D-Asp-Arg-Tyr-D-His-Lys
Val-D-Lys-His-Tyr-D-Phe-Ser-Tyr-Arg-NH₂
Gly-D-Phe-Lys-Tyr-His-D-Arg-Tyr-NH₂
Asp-D-Trp-Lys-Tyr-D-His-Phe-Arg-D-Gly-Lys-NH₂
D-His-Lys-Tyr-D-Phe-Glu-D-Asp-D-His-D-Lys-Arg-Trp-NH₂
H-Phe-D-Arg-Phe-Lys-Glu-Cys-Gly-NH₂
Phe-Arg-Phe-Lys-Glu-Cys-Gly
H-D-Arg-Dmt-Lys-Phe-Sar-Gly-Cys-NH₂

In another embodiment the peptide is defined by Formula III:

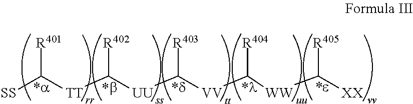

wherein:

one of SS and XX is

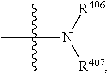

and the other is

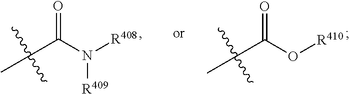

TT, UU, VV, and WW are each

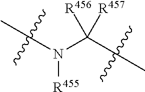

or TT, UU, VV, and WW are each

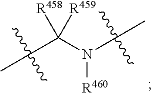

with the proviso when vv is 0 and uu is 1, one of SS and WW is

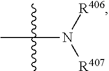

and the other of SS and WW is

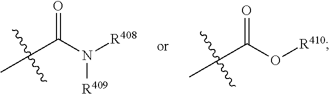

R⁴⁰¹ is

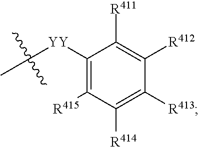

R⁴⁰² is

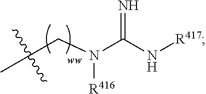

R⁴⁰³ is

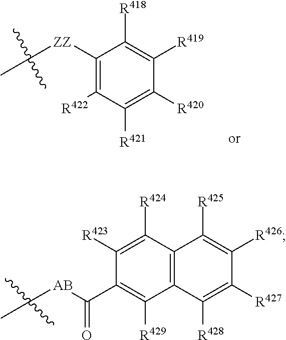

R⁴⁰⁴ is

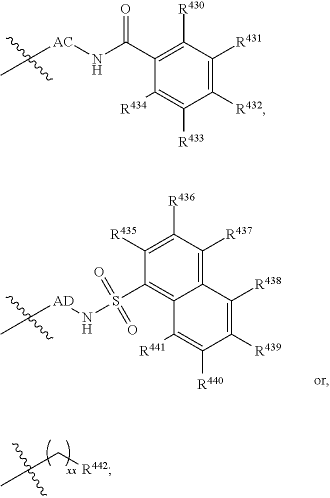

R⁴⁰⁵ is

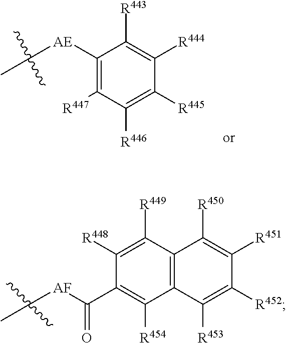

wherein
   [symbol] , [symbol] , [symbol] , [symbol] , and [symbol] each independently indicate carbon stereocenters, an absolute configuration of each of which is independently at each occurrence R or S;
   R⁴⁰⁶, R⁴⁰⁷, R⁴⁰⁸, R⁴⁰⁹, and R⁴¹⁰ are each independently a hydrogen or substituted or unsubstituted C₁-C₆ alkyl, C₂-C₆ alkenyl, C₂-C₆ alkynyl, saturated or unsaturated cycloalkyl, cycloalkylalkyl, aryl, aralkyl, 5- or 6-membered saturated or unsaturated heterocylyl, heterobicycyl, heteroaryl, or amino protecting group; or R⁴⁰⁶ and R⁴⁰⁷ together form a 3-, 4-, 5-, 6-, 7-, or 8-member substituted or unsubstituted heterocycyl ring;
   R⁴⁵⁵ and R⁴⁶⁰ are at each occurrence independently a hydrogen, —C(O)R^{e}, or an unsubstituted C₁-C₆ alkyl group;
   R⁴⁵⁶ and R⁴⁵⁷ are each independently a hydrogen or substituted or unsubstituted C₁-C₆ alkyl group; or together R⁴⁵⁶ and R⁴⁵⁷ are C═O;
   R⁴⁵⁸ and R⁴⁵⁹ are each independently a hydrogen or substituted or unsubstituted C₁-C₆ alkyl group; or together R⁴⁵⁸ and R⁴⁵⁹ are C═O;
   R⁴¹¹, R⁴¹², R⁴¹³, R⁴¹⁴, R⁴¹⁵, R⁴¹⁸, R⁴¹⁹, R⁴²⁰, R⁴²¹, R⁴²², R⁴²³, R⁴²⁴, R⁴²⁵, R⁴²⁶, R⁴²⁷, R⁴²⁸, R⁴²⁹, R⁴³⁰, R⁴³¹, R⁴³², R⁴³³, R⁴³⁴, R⁴³⁵, R⁴³⁶, R⁴³⁷, R⁴³⁸, R⁴³⁹, R⁴⁴⁰, R⁴⁴¹, R⁴⁴³, R⁴⁴⁴, R⁴⁴⁵, R⁴⁴⁶, R⁴⁴⁷, R⁴⁴⁸, R⁴⁴⁹, R⁴⁵⁰, R⁴⁵¹, R⁴⁵², R⁴⁵³, and R⁴⁵⁴ are each independently a hydrogen, deuterium, amino, amido, —NO₂, —CN, —OR^{e}, —SR^{e}, —NR^{e}R^{e}, —F, —Cl, —Br, —I, or a substituted or unsubstituted C₁-C₆ alkyl, C₁-C₆ alkoxy, —C(O)-alkyl, —C(O)-aryl, —C(O)-aralkyl, —C(O)₂R^{e}, C₁-C₄ alkylamino, C₁-C₄ dialkylamino, or perhaloalkyl group;
   R⁴¹⁶ and R⁴¹⁷ are each independently a hydrogen, —C(O)R^{e}, or a substituted or unsubstituted C₁-C₆ alkyl;
   R⁴⁴² is a hydrogen, —OR^{e}, —SR^{e}, —NR^{e}R^{e}, —NR^{e}R^{f}, —CO₂R^{e}, —C(O)NR^{e}R^{e}, —NR^{e}C(O)R^{e}, —NR^{e}C(NH)NH₂, —NR^{e}-dansyl, or a substituted or unsubstituted alkyl, aryl, or aralkyl group;
   YY, ZZ, and AE are each independently absent, —NH(CO)—, or —CH₂—;
   AB, AC, AD, and AF are each independently absent or C₁-C₆ alkylene group;
   R^{e} at each occurrence is independently a hydrogen or a substituted or unsubstituted C₁-C₆ alkyl group;
   R^{f} at each occurrence is independently a C₁-C₆ alkylene-NR^{e}-dansyl or C₁-C₆ alkylene-NR^{e}-anthraniloyl group;
   rr, ss, and vv are each independently 0 or 1; tt and uu are each 1 with the proviso that rr+ss+tt+uu+vv equals 4 or 5; and
   ww and xx are each independently 1, 2, 3, 4, or 5.

In some embodiments of peptides of Formula III,
R⁴⁰⁶ is a hydrogen, substituted or unsubstituted C₁-C₆ alkyl group,

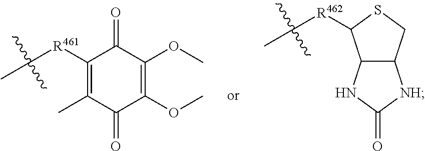

wherein R⁴⁶¹ is a —C₁-C₁₀ alkylene-CO₂— or —CO₂—C₁-C₁₀ alkylene-CO₂—; and R⁴⁶² is C₁-C₁₀ alkylene or C₁-C₁₀ alkylene-CO₂—;
R⁴⁰⁷, R⁴⁰⁸, R⁴⁰⁹, and R⁴¹⁰ are each independently a hydrogen or substituted or unsubstituted C₁-C₆ alkyl group;
R⁴⁵⁵ and R⁴⁶⁰ are each independently a hydrogen, —C(O)—C₁-C₆ alkyl, or methyl group;
R⁴⁵⁶ and R⁴⁵⁷ are each a hydrogen or together R⁴⁵⁶ and R⁴⁵⁷ are C═O;
R⁴⁵⁸ and R⁴⁵⁹ are each a hydrogen or together R⁴⁵⁸ and R⁴⁵⁹ are C═O;
R⁴¹⁶ and R⁴¹⁷ are each independently a hydrogen or —C(O)R^{e};
R⁴¹¹, R⁴¹², R⁴¹³, R⁴¹⁴, R⁴¹⁵, R⁴¹⁸, R⁴¹⁹, R⁴²⁰, R⁴²¹, R⁴²², R⁴⁴³, R⁴⁴⁴, R⁴⁴⁵, R⁴⁴⁶, and R⁴⁴⁷ are each independently a hydrogen, deuterium, methyl, or —OR^{e} group;
R⁴²³, R⁴²⁴, R⁴²⁵, R⁴²⁶, R⁴²⁷, R⁴²⁸, R⁴²⁹, R⁴³⁰, R⁴³¹, R⁴³², R⁴³³, R⁴³⁴, R⁴³⁵, R⁴³⁶, R⁴³⁷, R⁴³⁸, R⁴³⁹, R⁴⁴⁰, R⁴⁴¹, R⁴⁴⁸, R⁴⁴⁹, R⁴⁵⁰, R⁴⁵¹, R⁴⁵², R⁴⁵³, and R⁴⁵⁴ are each independently a hydrogen, NR^{e}R^{e}, or substituted or unsubstituted C₁-C₆ alkyl group;
R⁴⁴² is a —NR^{e}R^{e};
YY, ZZ, and AE are each independently absent or —CH₂—;
AB, AC, AD, and AF are each independently absent or C₁-C₄ alkylene group;
R^{e} at each occurrence is independently a hydrogen or a substituted or unsubstituted C₁-C₆ alkyl group;
rr, ss, and vv are each independently 0 or 1; tt and uu are each 1 with the proviso that rr+ss+tt+uu+vv equals 4 or 5; and
ww and xx are each independently 1, 2, 3, 4, or 5.

In some embodiments of peptides of Formula III,

### R⁴⁰⁶ is

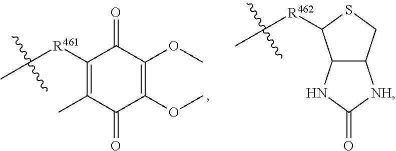

hydrogen, or methyl, wherein R⁴⁶¹ is —(CH₂)₃—CO₂—, —(CH₂)₉—CO₂—, or —CO₂—(CH₂)₂—CO₂— and R⁴⁶² is —(CH₂)₄—CO₂—;
R⁴⁰⁷, R⁴⁰⁸, R⁴⁰⁹, and R⁴¹⁰ are each a hydrogen or methyl group;
R⁴⁵⁵ and R⁴⁶⁰ are each independently a hydrogen, —C(O)CH₃, or methyl group;
R⁴⁵⁶ and R⁴⁵⁷ are each a hydrogen or together R⁴⁵⁶ and R⁴⁵⁷ are C═O;
R⁴⁵⁸ and R⁴⁵⁹ are each a hydrogen or together R⁴⁵⁸ and R⁴⁵⁹ are C═O;
R⁴¹⁶ and R⁴¹⁷ are each independently a hydrogen or —C(O)CH₃;
R⁴²⁶, R⁴³⁸, and R⁴⁵¹ are each —N(CH₃)₂;
R⁴³⁴ and R⁴⁴² are each —NH₂;
R⁴²³, R⁴²⁴, R⁴²⁵, R⁴²⁷, R⁴²⁸, R⁴²⁹, R⁴³⁰, R⁴³¹, R⁴³², R⁴³³, R⁴³⁵, R⁴³⁶, R⁴³⁷, R⁴³⁹, R⁴⁴⁰, R⁴⁴¹, R⁴⁴³, R⁴⁴⁴, R⁴⁴⁵, R⁴⁴⁶, R⁴⁴⁷, R⁴⁴⁸, R⁴⁴⁹, R⁴⁵⁰, R⁴⁵², R⁴⁵³, and R⁴⁵⁴ are each hydrogen;
R⁴¹², R⁴¹⁴, R⁴¹⁹, and R⁴²¹ are each independently hydrogen or deuterium;
R⁴¹¹, R⁴¹⁵, R⁴¹⁸, and R⁴²² are each independently hydrogen, deuterium, or methyl;
R⁴¹³ and R⁴²⁰ are each independently hydrogen, deuterium, or OR^{e};
YY, ZZ, and AE are each independently —CH₂—;
AB, AC, AD, and AF are each —CH₂— or a butylene group;
R^{e} at each occurrence is independently a hydrogen or a substituted or unsubstituted C₁-C₆ alkyl group;
rr, ss, and vv are each independently 0 or 1; tt and uu are each 1 with the proviso that rr+ss+tt+uu+vv equals 4 or 5; and
ww and xx are each independently 3 or 4.

In certain embodiments of Formula III,

SS is

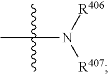

XX is

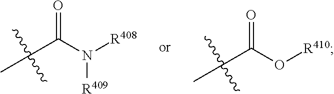

TT, UU, VV, and WW are each independently

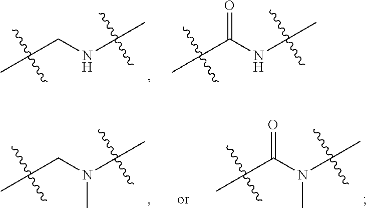

with the proviso when vv is 0 and uu is 1, WW is

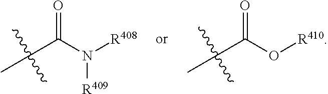

In some embodiments, the peptide of Formula III is selected from the peptides shown in Table D.

TABLE D

6-Butyric acid CoQ0-Phe-D-Arg-Phe-Lys-NH₂
6-Decanoic acid CoQ0-Phe-D-Arg-Phe-Lys-NH₂
H-D-N2-acetylarginine-Dmt-Lys-Phe-NH₂
H-D-N8-acetylarginine-Dmt-Lys-Phe-NH₂
H-N2-acetyl-D-arginine-L-Dmt-L-Lys-L-Phe-NH₂
H-N7-acetyl-D-arginine-Dmt-Lys-Phe-NH₂
H-Phe(d5)-D-Arg-Phe(d5)-Lys-NH₂
Succinic monoester CoQ0-Phe-D-Arg-Phe-Lys-HN₂
Dmt-D-Arg-Phe-(atn)Dap-NH₂
Dmt-D-Arg-Phe-(dns)Dap-NH₂
Dmt-D-Arg-A1d-Lys-NH₂
Dmt-D-Arg-Phe-Lys-Ald-NH₂
Bio-2′6′Dmt-D-Arg-Phe-Lys-NH₂
2′6′Dmt-D-Arg-Phe-dnsDap-NH₂
2′6′Dmt-D-Arg-Phe-atnDap-NH₂
H-D-Arg-Ψ[CH₂-NH]Dmt-Lys-Phe-NH₂
H-D-Arg-Dmt-Ψ[CH₂-NH]Lys-Phe-NH₂
H-D-Arg-Dmt-LysΨ[CH₂-NH]Phe-NH₂
H-D-Arg-Dmt-Ψ[CH₂-NH]Lys-Ψ[CH₂-NH]Phe-NH₂

In some embodiments, the peptide is selected from the peptides shown in Table E.

TABLE E

Arg-D-Leu-D-Tyr-Phe-Lys-Glu-D-Lys-Arg-D-Trp-Lys-
D-Phe-Tyr-D-Arg-Gly
Asp-Arg-D-Phe-Cys-Phe-D-Arg-D-Lys-Tyr-Arg-D-Tyr-
Trp-D-His-Tyr-D-Phe-Lys-Phe
D-Glu-Asp-Lys-D-Arg-D-His-Phe-Phe-D-Val-Tyr-Arg-
Tyr-D-Tyr-Arg-His-Phe-NH₂
Glu-Arg-D-Lys-Tyr-D-Val-Phe-D-His-Trp-Arg-D-Gly-
Tyr-Arg-D-Met-NH₂
Gly-Ala-Lys-Phe-D-Lys-Glu-Arg-Tyr-His-D-Arg-D-
Arg-Asp-Tyr-Trp-D-His-Trp-His-D-Lys-Asp
His-Tyr-D-Arg-Trp-Lys-Phe-D-Asp-Ala-Arg-Cys-D-
Tyr-His-Phe-D-Lys-Tyr-His-Ser-NH₂
Phe-Phe-D-Tyr-Arg-Glu-Asp-D-Lys-Arg-D-Arg-His-
Phe-NH₂
Phe-Tyr-Lys-D-Arg-Trp-His-D-Lys-D-Lys-Glu-Arg-D-
Tyr-Thr
Thr-Tyr-Arg-D-Lys-Trp-Tyr-Glu-Asp-D-Lys-D-Arg-
His-Phe-D-Tyr-Gly-Val-Ile-D-His-Arg-Tyr-Lys-NH₂
Tyr-Asp-D-Lys-Tyr-Phe-D-Lys-D-Arg-Phe-Pro-D-Tyr-
His-Lys
Tyr-D-His-Phe-D-Arg-Asp-Lys-D-Arg-His-Trp-D-
His-Phe
Phe-Tyr-Lys-D-Arg-Trp-His-D-Lys-D-Lys-Glu-Arg-D-
Tyr-Thr
Tyr-Asp-D-Lys-Tyr-Phe-D-Lys-D-Arg-Phe-Pro-D-Tyr-
His-Lys
Glu-Arg-D-Lys-Tyr-D-Val-Phe-D-His-Trp-Arg-D-Gly-
Tyr-Arg-D-Met-NH₂
Arg-D-Leu-D-Tyr-Phe-Lys-Glu-D-Lys-Arg-D-Trp-Lys-
D-Phe-Tyr-D-Arg-Gly
Gly-Ala-Lys-Phe-D-Lys-Glu-Arg-Tyr-His-D-Arg-D-
Arg-Asp-Tyr-Trp-D-His-Trp-His-D-Lys-Asp
Gly-Ala-Lys-Phe-D-Lys-Glu-Arg-Tyr-His-D-Arg-D-
Arg-Asp-Tyr-Trp-D-His-Trp-His-D-Lys-Asp

In one embodiment, the aromatic-cationic peptides of the present technology have a core structural motif of alternating aromatic and cationic amino acids. For example, the peptide may be a tetrapeptide defined by any of Formulas A to F set forth below:

Aromatic-Cationic-Aromatic-Cationic (Formula A)

Cationic-Aromatic-Cationic-Aromatic (Formula B)

Aromatic-Aromatic-Cationic-Cationic (Formula C)

Cationic-Cationic-Aromatic-Aromatic (Formula D)

Aromatic-Cationic-Cationic-Aromatic (Formula E)

Cationic-Aromatic-Aromatic-Cationic (Formula F)

wherein, Aromatic is a residue selected from the group consisting of: Phe (F), Tyr (Y), and Trp (W). In some embodiments, the Aromatic residue may be substituted with a saturated analog of an aromatic residue, e.g., Cyclohexylalanine (Cha). In some embodiments, Cationic is a residue selected from the group consisting of: Arg (R), Lys (K), and His (H).

The amino acids of the aromatic-cationic peptides of the present technology can be any amino acid. As used herein, the term “amino acid” is used to refer to any organic molecule that contains at least one amino group and at least one carboxyl group. In some embodiments, at least one amino group is at the a position relative to the carboxyl group.

The amino acids may be naturally occurring. Naturally occurring amino acids include, for example, the twenty most common levorotatory (L,) amino acids normally found in mammalian proteins, i.e., alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan, (Trp), tyrosine (Tyr), and valine (Val).

Other naturally occurring amino acids include, for example, amino acids that are synthesized in metabolic processes not associated with protein synthesis. For example, the amino acids ornithine and citrulline are synthesized in mammalian metabolism during the production of urea.

The peptides useful in the present technology can contain one or more non-naturally occurring amino acids. The non-naturally occurring amino acids may be (L-), dextrorotatory (D), or mixtures thereof. In some embodiments, the peptide has no amino acids that are naturally occurring.

Non-naturally occurring amino acids are those amino acids that typically are not synthesized in normal metabolic processes in living organisms, and do not naturally occur in proteins. In certain embodiments, the non-naturally occurring amino acids useful in the present technology are also not recognized by common proteases.

The non-naturally occurring amino acid can be present at any position in the peptide. For example, the non-naturally occurring amino acid can be at the N terminus, the C-terminus, or at any position between the N-terminus and the C-terminus.

The non-natural amino acids may, for example, comprise alkyl, aryl, or alkylaryl groups. Some examples of alkyl amino acids include α-aminobutyric acid, β-aminobutyric acid, γ-aminobutyric acid, δ-aminovaleric acid, and ε-aminocaproic acid. Some examples of aryl amino acids include ortho-, meta, and para-aminobenzoic acid. Some examples of alkylaryl amino acids include ortho-, meta-, and para-aminophenyl acetic acid, and γ-phenyl-β-aminobutyric acid.

Non-naturally occurring amino acids also include derivatives of naturally occurring amino acids. The derivatives of naturally occurring amino acids may, for example, include the addition of one or more chemical groups to the naturally occurring amino acid.

For example, one or more chemical groups can be added to one or more of the 2′, 3′, 4′, 5′, or 6′ position of the aromatic ring of a phenylalanine or tyrosine residue, or the 4′, 5′, 6′, or 7′ position of the benzo ring of a tryptophan residue. The group can be any chemical group that can be added to an aromatic ring. Some examples of such groups include branched or unbranched C₁-C₄ alkyl, such as methyl, ethyl, n-propyl, isopropyl, butyl, isobutyl, or t-butyl, C₁-C₄ alkyloxy (i.e., alkoxy), amino, C₁-C₄ alkylamino and C₁-C₄ dialkylamino (e.g., methylamino, dimethylamino), nitro, hydroxyl, halo (i.e., fluoro, chloro, bromo, or iodo). Some specific examples of non-naturally occurring derivatives of naturally occurring amino acids include norvaline (Nva), norleucine (Nle), and hydroxyproline (Hyp).

Another example of a modification of an amino acid in a peptide useful in the present methods is the derivatization of a carboxyl group of an aspartic acid or a glutamic acid residue of the peptide. One example of derivatization is amidation with ammonia or with a primary or secondary amine, e.g., methylamine, ethylamine, dimethylamine or diethylamine. Another example of derivatization includes esterification with, for example, methyl or ethyl alcohol.

Another such modification includes derivatization of an amino group of a lysine, arginine, or histidine residue. For example, such amino groups can be alkylated or acylated. Some suitable acyl groups include, for example, a benzoyl group or an alkanoyl group comprising any of the C₁-C₄ alkyl groups mentioned above, such as an acetyl or propionyl group.

In some embodiments, the non-naturally occurring amino acids are resistant, and in some embodiments insensitive, to common proteases. Examples of non-naturally occurring amino acids that are resistant or insensitive to proteases include the dextrorotatory (D-) form of any of the above-mentioned naturally occurring L-amino acids, as well as L- and/or D non-naturally occurring amino acids. The D-amino acids do not normally occur in proteins, although they are found in certain peptide antibiotics that are synthesized by means other than the normal ribosomal protein synthetic machinery of the cell, as used herein, the D-amino acids are considered to be non-naturally occurring amino acids.

In order to minimize protease sensitivity, the peptides useful in the methods of the present technology should have less than five, less than four, less than three, or less than two contiguous L-amino acids recognized by common proteases, irrespective of whether the amino acids are naturally or non-naturally occurring. In some embodiments, the peptide has only D-amino acids, and no L-amino acids.

If the peptide contains protease sensitive sequences of amino acids, at least one of the amino acids is a non-naturally-occurring D-amino acid, thereby conferring protease resistance. An example of a protease sensitive sequence includes two or more contiguous basic amino acids that are readily cleaved by common proteases, such as endopeptidases and trypsin. Examples of basic amino acids include arginine, lysine and histidine. In some embodiments, at least one of the amides in the peptide backbone are alkylated, thereby conferring protease resistance.

It is important that the aromatic-cationic peptides have a minimum number of net positive charges at physiological pH in comparison to the total number of amino acid residues in the peptide. The minimum number of net positive charges at physiological pH is referred to below as (pₘ). The total number of amino acid residues in the peptide is referred to below as (r).

The minimum number of net positive charges discussed below are all at physiological pH. The term “physiological pH” as used herein refers to the normal pH in the cells of the tissues and organs of the mammalian body. For instance, the physiological pH of a human is normally approximately 7.4, but normal physiological pH in mammals may be any pH from about 7.0 to about 7.8.

Typically, a peptide has a positively charged N-terminal amino group and a negatively charged C-terminal carboxyl group. The charges cancel each other out at physiological pH. As an example of calculating net charge, the peptide Tyr-Arg-Phe-Lys-Glu-His-Trp-Arg has one negatively charged amino acid (i.e., Glu) and four positively charged amino acids (i.e., two Arg residues, one Lys, and one His). Therefore, the above peptide has a net positive charge of three.

In one embodiment, the aromatic-cationic peptides have a relationship between the minimum number of net positive charges at physiological pH (pₘ) and the total number of amino acid residues (r) wherein 3pₘ is the largest number that is less than or equal to r+1. In this embodiment, the relationship between the minimum number of net positive charges (pₘ) and the total number of amino acid residues (r) is as follows:

TABLE 1
Amino acid number and net positive charges (3pₘ ≤ p + 1)

| (r) | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|
| (pₘ) | 1 | 1 | 2 | 2 | 2 | 3 | 3 | 3 | 4 | 4 | 4 | 5 | 5 | 5 | 6 | 6 | 6 | 7 |

In another embodiment, the aromatic-cationic peptides have a relationship between the minimum number of net positive charges (pₘ) and the total number of amino acid residues (r) wherein 2pₘ is the largest number that is less than or equal to r+1. In this embodiment, the relationship between the minimum number of net positive charges (pₘ) and the total number of amino acid residues (r) is as follows:

TABLE 2
Amino acid number and net positive charges (2pₘ ≤ p + 1)

| (r) | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|
| (pₘ) | 2 | 2 | 3 | 3 | 4 | 4 | 5 | 5 | 6 | 6 | 7 | 7 | 8 | 8 | 9 | 9 | 10 | 10 |

In one embodiment, the minimum number of net positive charges (pₘ) and the total number of amino acid residues (r) are equal. In another embodiment, the peptides have three or four amino acid residues and a minimum of one net positive charge, or a minimum of two net positive charges, or a minimum of three net positive charges.

It is also important that the aromatic-cationic peptides have a minimum number of aromatic groups in comparison to the total number of net positive charges (pₜ). The minimum number of aromatic groups will be referred to below as (a). Naturally-occurring amino acids that have an aromatic group include the amino acids histidine, tryptophan, tyrosine, and phenylalanine. For example, the hexapeptide Lys-Gln-Tyr-D-Arg-Phe-Trp has a net positive charge of two (contributed by the lysine and arginine residues) and three aromatic groups (contributed by tyrosine, phenylalanine and tryptophan residues).

The aromatic-cationic peptides should also have a relationship between the minimum number of aromatic groups (a) and the total number of net positive charges at physiological pH (pₜ) wherein 3a is the largest number that is less than or equal to pₜ+1, except that when pₜ is 1, a may also be 1. In this embodiment, the relationship between the minimum number of aromatic groups (a) and the total number of net positive charges (pₜ) is as follows:

TABLE 3
Aromatic groups and net positive charges (3a ≤ pₜ + 1 or a = pₜ = 1)

| (pₜ) | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| (a) | 1 | 1 | 1 | 1 | 2 | 2 | 2 | 3 | 3 | 3 | 4 | 4 | 4 | 5 | 5 | 5 | 6 | 6 | 6 | 7 |

In another embodiment, the aromatic-cationic peptides have a relationship between the minimum number of aromatic groups (a) and the total number of net positive charges (pₜ) wherein 2a is the largest number that is less than or equal to pₜ+1. In this embodiment, the relationship between the minimum number of aromatic amino acid residues (a) and the total number of net positive charges (pₜ) is as follows:

TABLE 4
Aromatic groups and net positive charges (2a ≤ pₜ + 1 or a = pₜ = 1)

| (pₜ) | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| (a) | 1 | 1 | 2 | 2 | 3 | 3 | 4 | 4 | 5 | 5 | 6 | 6 | 7 | 7 | 8 | 8 | 9 | 9 | 10 | 10 |

In another embodiment, the number of aromatic groups (a) and the total number of net positive charges (pt) are equal.

In some embodiments, carboxyl groups, especially the terminal carboxyl group of a C-terminal amino acid, are amidated with, for example, ammonia to form the C-terminal amide. Alternatively, the terminal carboxyl group of the C-terminal amino acid may be amidated with any primary or secondary amine. The primary or secondary amine may, for example, be an alkyl, especially a branched or unbranched C₁-C₄ alkyl, or an aryl amine. Accordingly, the amino acid at the C-terminus of the peptide may be converted to an amido, N-methylamido, N-ethylamido, N,N-dimethylamido, N,N-diethyl amido, N-methyl-N-ethylamido, N-phenylamido or N-phenyl-N-ethylamido group.

The free carboxylate groups of the asparagine, glutamine, aspartic acid, and glutamic acid residues not occurring at the C-terminus of the aromatic-cationic peptides of the present technology may also be amidated wherever they occur within the peptide. The amidation at these internal positions may be with ammonia or any of the primary or secondary amines described herein.

In one embodiment, the aromatic-cationic peptide useful in the methods of the present technology is a tripeptide having two net positive charges and at least one aromatic amino acid. In a particular embodiment, the aromatic-cationic peptide useful in the methods of the present technology is a tripeptide having two net positive charges and two aromatic amino acids.

In some embodiments, the aromatic-cationic peptide is a peptide having:

at least one net positive charge;

a minimum of four amino acids;

a maximum of about twenty amino acids;

a relationship between the minimum number of net positive charges (pₘ) and the total number of amino acid residues (r) wherein 3pₘ is the largest number that is less than or equal to r+1; and a relationship between the minimum number of aromatic groups (a) and the total number of net positive charges (pₜ) wherein 2a is the largest number that is less than or equal to pₜ+1, except that when a is 1, pₜ may also be 1.

In one embodiment, 2pₘ is the largest number that is less than or equal to r+1, and a may be equal to pₜ. The aromatic-cationic peptide may be a water-soluble peptide having a minimum of two or a minimum of three positive charges.

In one embodiment, the peptide comprises one or more non-naturally occurring amino acids, for example, one or more D-amino acids. In some embodiments, the C-terminal carboxyl group of the amino acid at the C-terminus is amidated. In certain embodiments, the peptide has a minimum of four amino acids. The peptide may have a total of about 6, a total of about 9, or a total of about 12 amino acids.

In one embodiment, the peptides have a tyrosine residue or a tyrosine derivative at the N-terminus (i.e., the first amino acid position). Suitable derivatives of tyrosine include 2′-methyltyrosine (Mmt); 2′,6′-dimethyltyrosine (2′6′-Dmt); 3′,5′-dimethyltyrosine (3′5′Dmt); N,2′,6′-trimethyltyrosine (Tmt); and 2′-hydroxy-6′-methyltyrosine (Hmt).

In one embodiment, a peptide has the formula Tyr-D-Arg-Phe-Lys-NH₂. Tyr-D-Arg-Phe-Lys-NH₂ has a net positive charge of three, contributed by the amino acids tyrosine, arginine, and lysine and has two aromatic groups contributed by the amino acids phenylalanine and tyrosine. The tyrosine of Tyr-D-Arg-Phe-Lys-NH₂ can be a modified derivative of tyrosine such as in 2′6′-dimethyltyrosine to produce the compound having the formula 2′6′-Dmt-D-Arg-Phe-Lys-NH₂. 2′6′-Dmt-D-Arg-Phe-Lys-NH₂ has a molecular weight of 640 and carries a net three positive charge at physiological pH. 2′6′-Dmt-D-Arg-Phe-Lys-NH₂ readily penetrates the plasma membrane of several mammalian cell types in an energy-independent manner (Zhao et al., J. Pharmacol Exp Ther., 304:425-432, 2003).

Alternatively, in some embodiments, the aromatic-cationic peptide does not have a tyrosine residue or a derivative of tyrosine at the N-terminus (i.e., amino acid position 1). The amino acid at the N-terminus can be any naturally-occurring or non-naturally-occurring amino acid other than tyrosine. In one embodiment, the amino acid at the N-terminus is phenylalanine or its derivative. Exemplary derivatives of phenylalanine include 2′-methylphenylalanine (Mmp), 2′,6′-dimethylphenylalanine (2′,6′-Dmp), N,2′,6′-trimethylphenylalanine (Tmp), and 2′-hydroxy-6′-methylphenylalanine (Hmp).

An example of an aromatic-cationic peptide that does not have a tyrosine residue or a derivative of tyrosine at the N-terminus is a peptide with the formula Phe-D-Arg-Phe-Lys-NH₂. Alternatively, the N-terminal phenylalanine can be a derivative of phenylalanine such as 2′6′-dimethylphenylalanine (2′6′-Dmp). In one embodiment, the amino acid sequence of 2′6′-Dmt-D-Arg-Phe-Lys-NH₂ is rearranged such that Dmt is not at the N-terminus. An example of such an aromatic-cationic peptide is a peptide having the formula of D-Arg-2′6′-Dmt-Lys-Phe-NH₂.

Suitable substitution variants of the peptides listed herein include conservative amino acid substitutions. Amino acids may be grouped according to their physicochemical characteristics as follows:

(a) Non-polar amino acids: Ala(A) Ser(S) Thr(T) Pro(P) Gly(G) Cys (C);

(b) Acidic amino acids: Asn(N) Asp(D) Glu(E) Gln(Q);

(c) Basic amino acids: His(H) Arg(R) Lys(K);

(d) Hydrophobic amino acids: Met(M) Leu(L) Ile(I) Val(V); and

(e) Aromatic amino acids: Phe(F) Tyr(Y) Trp(W).

Substitutions of an amino acid in a peptide by another amino acid in the same group are referred to as a conservative substitution and may preserve the physicochemical characteristics of the original peptide. In contrast, substitutions of an amino acid in a peptide by another amino acid in a different group are generally more likely to alter the characteristics of the original peptide.

The amino acids of the peptides disclosed herein may be in either the L- or the D-configuration.

In some embodiments, the aromatic-cationic peptides disclosed herein, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis or tri-acetate salt, a tartrate salt, a fumarate salt, a mono, bis or tri-HCl salt, a mono, bis, or tri-tosylate salt, or a mono, bis or tri-trifluoroacetate salt) are for use in treating or preventing TON in a subject in need thereof. In some embodiments, the aromatic-cationic peptide is D-Arg-2′,6′-Dmt-Lys-Phe-NH₂, or a pharmaceutically acceptable salt thereof. In some embodiments, the subject has been diagnosed as having TON.

In other embodiments, the aromatic-cationic peptides disclosed herein, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis or tri-acetate salt, a tartrate salt, a fumarate salt, a mono, bis, or tri-HCl salt, a mono, bis or tri-tosylate salt, or a mono, bis or tri-trifluoroacetate salt) are for use in improving visual function in a subject having TON. In some embodiments, the aromatic-cationic peptide is D-Arg-2′,6′-Dmt-Lys-Phe-NH₂, or a pharmaceutically acceptable salt thereof. In some embodiments, the subject has been diagnosed as having TON.

In some embodiments, the aromatic-cationic peptides disclosed herein, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis or tri-acetate salt, a tartrate salt, a fumarate salt, a mono, bis, or tri-HCl salt, a mono, bis or tri-tosylate salt, or a mono, bis or tri-trifluoroacetate salt) are for use in promoting retinal ganglion cell (RGC) survival or increasing neurite outgrowth of an RGC. In some embodiments, the aromatic-cationic peptide is D-Arg-2′,6′-Dmt-Lys-Phe-NH₂, or a pharmaceutically acceptable salt thereof.

In some embodiments of the peptides of the present technology, the TON is caused by direct injury or indirect injury to the subject. In some embodiments, the direct or indirect injury is selected from the group consisting of intraorbital injury, intracanalicular injury, intracranial injury, and an injury to the subject's optic nerve.

In some embodiments of the peptides of the present technology, the peptide is intended to be administered prior to injury. In some embodiments, the peptide is intended to be administered immediately following injury. In some embodiments, the peptide is intended to be administered about 10 minutes or less, 20 minutes or less, about 30 minutes or less, about 40 minutes or less, about 50 minutes or less, about 1 hour or less, about 1.5 hours or less, about 2 hours or less, about 3 hours or less, about 4 hours or less, about 5 hours or less, about 6 hours or less, about 7 hours less, about 8 hours or less, about 9 hours or less, about 10 hours or less, about 11 hours or less, about 12 hours or less, about 16 hours or less, about 20 hours or less, about 24 hours or less, about 36 hours or less, about 48 hours or less, about 72 hours or less, about 96 hours or less, about 5 days or less, about 6 days or less, or about one week or less following the injury. In some embodiments, the peptide is intended to be administered daily for about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks or more. In some embodiments, the peptide is intended to be administered daily for 16 weeks or more.

In some embodiments of the peptides of the present technology, the treating or preventing comprises the treatment or prevention of one or more signs or symptoms of TON comprising one or more of vision loss, blurred vision, RGC damage, scotoma, decreased color sensation, uveitis, optic neuritis, eye pain, optic nerve avulsion, optic nerve transection, optic nerve sheath hemorrhage, orbital hemorrhage, choroidal rupture, and commotio retinae.

In some embodiments of the peptides of the present technology, the peptide is intended or formulated to be administered to the subject or the RGC separately, sequentially, or simultaneously with an additional therapeutic agent or an additional therapeutic treatment. In some embodiments, the additional therapeutic agent is selected from the group consisting of: TNFα inhibitor, corticosteroid, IL-1R antagonist, resveratrol, potassium channel blocker, and necrostatin-1. In some embodiments, the TNFα inhibitor is etanercept. In some embodiments, the potassium channel blocker is 4-aminopyridine (4-AP). In some embodiments, the additional therapeutic treatment is a therapeutic cooling treatment that reduces the subject's temperature. In some embodiments, the additional therapeutic treatment induces hypothermia in the subject. In some embodiments, the peptides are for use wherein the combination of peptide and an additional therapeutic agent or treatment has a synergistic effect in the prevention or treatment of TON. In some embodiments, the peptides are for use wherein the combination of peptide and an additional therapeutic agent has a synergistic effect in in promoting RGC survival or increasing neurite outgrowth of an RGC.

### Synthesis of Aromatic-Cationic Peptides

The aromatic-cationic peptides disclosed herein (such as 2′6′-Dmt-D-Arg-Phe-Lys-NH₂, Phe-D-Arg-Phe-Lys-NH₂, or D-Arg-2′6′-Dmt-Lys-Phe-NH₂) may be synthesized by any method known in the art. Exemplary, nonlimiting methods for chemically synthesizing peptides include those described by Stuart and Young in “Solid Phase Peptide Synthesis,” Second Edition, Pierce Chemical Company (1984), in “Solid Phase Peptide Synthesis,” Methods Enzymol. 289, Academic Press, Inc., New York (1997) and N. Leo Benoit, “Chemistry of Peptide Synthesis” CRC Press, Boca Raton, 2006. Additional methods suitable for preparing peptides described herein, such as D-Arg-2′6′-Dmt-Lys-Phe-NH₂ and its representative salt forms, may be found in the following published patent applications: WO2004/070054, WO2017/156403, WO2018/034901 and WO2018/187400.

Recombinant peptides may be generated using conventional techniques in molecular biology, protein biochemistry, cell biology, and microbiology, such as those described in Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, N Y, 1987); and Meth. Enzymol., Vols. 154 and 155, Wu & Grossman, and Wu, Eds., respectively.

Aromatic-cationic peptide precursors may be made by either chemical (e.g., using solution and solid phase chemical peptide synthesis) or recombinant syntheses known in the art. Precursors of e.g., amidated aromatic-cationic peptides of the present technology may be made in like manner. In some embodiments, recombinant production is believed significantly more cost effective. In some embodiments, precursors are converted to active peptides by amidation reactions that are also known in the art. For example, enzymatic amidation is described in U.S. Pat. No. 4,708,934 and European Patent Publications 0 308 067 and 0 382 403. Recombinant production can be used for both the precursor and the enzyme that catalyzes the conversion of the precursor to the desired active form of the aromatic-cationic peptide. Such recombinant production is discussed in Biotechnology, Vol. 11 (1993) pp. 64-70, which further describes a conversion of a precursor to an amidated product. During amidation, a keto-acid such as an alpha-keto acid, or salt or ester thereof, wherein the alpha-keto acid has the molecular structure RC(O)C(O)OH, and wherein R is selected from the group consisting of aryl, a C₁-C₄ hydrocarbon moiety, a halogenated or hydroxylated C₁-C₄ hydrocarbon moiety, and a C₁-C₄ carboxylic acid, may be used in place of a catalase co-factor. Examples of these keto acids include, but are not limited to, ethyl pyruvate, pyruvic acid and salts thereof, methyl pyruvate, benzoyl formic acid and salts thereof, 2-ketobutyric acid and salts thereof, 3-methyl-2-oxobutanoic acid and salts thereof, and 2-keto glutaric acid and salts thereof.

In some embodiments, the production of the recombinant aromatic-cationic peptide may proceed, for example, by producing glycine-extended precursor in E. coli as a soluble fusion protein with glutathione-S-transferase. An α-amidating enzyme catalyzes conversion of precursors to active aromatic-cationic peptide. That enzyme is recombinantly produced, for example, in Chinese Hamster Ovary (CHO) cells as described in the Biotechnology article cited above. Other precursors to other amidated peptides may be produced in like manner. Peptides that do not require amidation or other additional functionalities may also be produced in like manner. Other peptide active agents are commercially available or may be produced by techniques known in the art.

### Therapeutic Methods

The following discussion is presented by way of example only, and is not intended to be limiting.

One aspect of the present technology includes methods of treating TON in a subject diagnosed as having, suspected as having, or at risk of having TON. In therapeutic applications, compositions or medicaments comprising an aromatic-cationic peptide, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) are administered to a subject suspected of, or already suffering from an optic neuropathy in an amount sufficient to cure, or at least partially arrest, the symptoms of the disease, including its complications and intermediate pathological phenotypes in development of the disease. In some embodiments of the methods of the present technology, the aromatic-cationic peptide is D-Arg-2′,6′-Dmt-Lys-Phe-NH₂, or a pharmaceutically acceptable salt thereof.

Another aspect of the present technology includes methods of improving visual function in a subject diagnosed as having, suspected as having, or at risk of having TON. In therapeutic applications, compositions or medicaments comprising an aromatic-cationic peptide, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis or tri-acetate salt, a tartrate salt, a fumarate salt, a mono, bis or tri-HCl salt, a mono, bis or tri-tosylate salt, or a mono, bis or tri-trifluoroacetate salt), are administered to a subject suspected of, or already suffering from an optic neuropathy in an amount sufficient to improve visual function in the subject. In some embodiments of the methods of the present technology, the aromatic-cationic peptide is D-Arg-2′,6′-Dmt-Lys-Phe-NH₂, or a pharmaceutically acceptable salt thereof.

One aspect of the present technology includes methods of promoting retinal ganglion cell (RGC) survival or increasing neurite outgrowth of an RGC. In some embodiments, the RGC is in a subject having, suspected as having, or at risk of having TON. In therapeutic applications, compositions or medicaments comprising an aromatic-cationic peptide, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) are administered to a subject suspected of, or already suffering from TON in an amount sufficient to improve visual function in the subject. In some embodiments of the methods of the present technology, the aromatic-cationic peptide is D-Arg-2′,6′-Dmt-Lys-Phe-NH₂, or a pharmaceutically acceptable salt thereof.

Other aspects of the present technology include uses of a composition in the preparation of a medicament for treating or preventing TON in a subject in need thereof, uses of a composition in the preparation of a medicament for improving visual function in a subject having or suspected of having TON, and uses of a composition in the preparation of a medicament for promoting RGC survival or increasing neurite outgrowth of an RGC. The compositions or medicaments comprising an aromatic-cationic peptide, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) are suitable for administration to a subject suspected of, or already suffering from TON in an amount sufficient to improve visual function in the subject. In some embodiments of the methods of the present technology, the aromatic-cationic peptide is D-Arg-2′,6′-Dmt-Lys-Phe-NH₂, or a pharmaceutically acceptable salt thereof.

Subjects suffering from an optic neuropathy such as TON can be identified by any or a combination of diagnostic or prognostic assays known in the art. For example, typical symptoms of TON include, but are not limited to, vision loss, blurred vision, RGC damage, scotoma, decreased color sensation, uveitis, optic neuritis, eye pain, optic nerve avulsion, optic nerve transection, optic nerve sheath hemorrhage, orbital hemorrhage, choroidal rupture, and commotio retinae. In some embodiments, subjects suffering from TON have a direct or indirect injury. In some embodiments, the injury is selected from the group consisting of intraorbital injury, intracanalicular injury, intracranial injury, and an injury to the subject's optic nerve. In some embodiments, a subject suffering TON is identified by damage to the subject's RGCs, as detected by BVCA, PERG, ERG, STR, PhNR, OCT, VEP, and/or prVEP, as described herein.

For therapeutic applications, a composition comprising an aromatic-cationic peptide, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) is administered to the subject. In some embodiments, the peptide composition is administered one, two, three, four, or five times per day. In some embodiments, the peptide composition is administered more than five times per day. Additionally or alternatively, in some embodiments, the peptide composition is administered every day, every other day, every third day, every fourth day, every fifth day, or every sixth day. In some embodiments, the peptide composition is administered weekly, bi-weekly, tri-weekly, or monthly. In some embodiments, the peptide composition is administered for a period of one, two, three, four, or five weeks. In some embodiments, the peptide is administered for six weeks or more. In some embodiments, the peptide is administered for twelve weeks or more. In some embodiments, the peptide is administered for a period of less than one year. In some embodiments, the peptide is administered for a period of more than one year or until vision is all or partially restored in the subject.

The subject treated in accordance with the present therapeutic methods can be any mammal, including, for example, farm animals, such as sheep, pigs, cows, and horses; pet animals, such as dogs and cats; laboratory animals, such as rats, mice and rabbits. In some embodiments, the mammal is a human.

In some embodiments, treatment of subjects diagnosed with or suspected of having an TON with one or more aromatic-cationic peptides ameliorates or eliminates of one or more of the following symptoms of TON: RGC damage, vision loss, blurred vision, RGC damage, scotoma, decreased color sensation, uveitis, optic neuritis, eye pain, optic nerve avulsion, optic nerve transection, optic nerve sheath hemorrhage, orbital hemorrhage, choroidal rupture, and commotio retinae. In some embodiments, treatment success with one or more aromatic-cationic peptides is determined by detecting an improvement in the subject's RGCs compared to one or more of: (1) a baseline measurement or level of damage detected prior to or with commencement of treatment; (2) a measurement or level of damage in an unaffected (contralateral) eye that does not exhibit one or more symptoms of TON; (3) a measurement or level of damage from a control subject or a population of control subjects, wherein the control subjects exhibit one or more symptoms of optic neuropathy and either (i) have not been administered an aromatic-cationic peptide, or (ii) have been administered a control peptide; or (4) a standard. In some embodiments, improvements in the subject's RGCs are detected by one or more of BVCA, PERG, ERG, STR, PhNR, OCT, VEP, and/or prVEP, as described herein.

### Prophylactic Methods

In one aspect, the present technology provides a method for preventing or delaying the onset of TON or one or more symptoms of TON in a subject at risk of having or developing TON. In prophylactic applications, pharmaceutical compositions or medicaments of aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis or tri-trifluoroacetate salt) are administered to a subject susceptible to, or otherwise at risk of for TON in an amount sufficient to eliminate or reduce the risk, or delay the onset of the disease, including biochemical, histologic and/or behavioral symptoms of the disease, its complications and intermediate pathological phenotypes presenting during development of the disease. In some embodiments of the methods of the present technology, the aromatic-cationic peptide is D-Arg-2′,6′-Dmt-Lys-Phe-NH₂, or a pharmaceutically acceptable salt thereof.

Administration of a prophylactic aromatic-cationic peptide can occur prior to the manifestation of symptoms characteristic of the disease or disorder, such that the disease or disorder is prevented or, alternatively, delayed in its progression.

Subjects at risk for an optic neuropathy can be identified by, e.g., any or a combination of diagnostic or prognostic assays known in the art. In some embodiments, subjects at risk for TON are subjects that have experienced a traumatic injury. In some embodiments, the traumatic injury is a direct injury or an indirect injury. In some embodiments, the direct or indirect injury is selected from the group consisting of intraorbital injury, intracanalicular injury, intracranial injury, and an injury to the subject's optic nerve.

For prophylactic applications, a composition comprising an aromatic-cationic peptide, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) is administered to the subject. In some embodiments, the peptide composition is administered one, two, three, four, or five times per day. In some embodiments, the peptide composition is administered more than five times per day. Additionally or alternatively, in some embodiments, the peptide composition is administered every day, every other day, every third day, every fourth day, every fifth day, or every sixth day. In some embodiments, the peptide composition is administered weekly, bi-weekly, tri-weekly, or monthly. In some embodiments, the peptide composition is administered for a period of one, two, three, four, or five weeks. In some embodiments, the peptide is administered for six weeks or more. In some embodiments, the peptide is administered for twelve weeks or more. In some embodiments, the peptide is administered for a period of less than one year. In some embodiments, the peptide is administered for a period of more than one year.

In some embodiments, treatment with the aromatic-cationic peptide will prevent or delay the onset of one or more of the following symptoms: vision loss, blurred vision, RGC damage, scotoma, decreased color sensation, uveitis, optic neuritis, eye pain, optic nerve avulsion, optic nerve transection, optic nerve sheath hemorrhage, orbital hemorrhage, choroidal rupture, and commotio retinae.

The mammal treated in accordance with the present prophylactic methods can be any mammal, including, for example, farm animals, such as sheep, pigs, cows, and horses; pet animals, such as dogs and cats; laboratory animals, such as rats, mice and rabbits. In some embodiments, the mammal is a human.

### Determination of the Biological Effect of the Aromatic-Cationic Peptide-Based Therapeutic

In various embodiments, suitable in vitro or in vivo assays are performed to determine the effect of a specific aromatic-cationic peptide-based therapeutic and whether its administration is indicated for treatment. In various embodiments, in vitro assays can be performed with representative animal models, to determine if a given aromatic-cationic peptide-based therapeutic exerts the desired effect on reducing or eliminating signs and/or symptoms of TON.

### Animal Models

Compounds for use in therapy can be tested in suitable animal model systems including, but not limited to rats, mice, chicken, cows, monkeys, rabbits, and the like, prior to testing in human subjects. Similarly, for in vivo testing, any of the animal model systems known in the art can be used prior to administration to human subjects. In some embodiments, in vitro or in vivo testing is directed to the biological function of 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt). In some embodiments of the methods of the present technology, the aromatic-cationic peptide is D-Arg-2′,6′-Dmt-Lys-Phe-NH₂, or a pharmaceutically acceptable salt thereof.

Animal models of optic neuropathy may be generated using techniques known in the art. Animal models of TON include, but are not limited to: (i) sonication induced TON (SI-TON) that closely recapitulates the clinical manifestations of indirect TON; (ii) optic nerve crush-induced TON (ONC-TON), which is a more severe form of TON often resulting in axtomized nerve fibers and ruptured nervous vasculature; and (iii) ocular blast models. Such models may be used to demonstrate the biological effect of aromatic-cationic peptides of the present technology, such as 2′6′-Dmt-D-Arg-Phe-Lys-NH₂, Phe-D-Arg-Phe-Lys-NH₂, or D-Arg-2′6′-Dmt-Lys-Phe-NH₂, in the prevention and treatment of optic neuropathies such as TON, and for determining what comprises a therapeutically effective amount of peptide in a given context.

SI-TON is induced by a microtip probe sonifier placed on the supraorbital ridge directly above the entrance of the optic nerve into the bony canal, as described in Tao, W. et al., Scientific Reports (2017) 7: 11779 (incorporated herein by reference). An ultrasonic pulse is then delivered to the optic nerve. After injury, the number of RGCs in the retina as well as visual function measured by PERG steadily decrease over a two-week period. In the optic nerve, pro-inflammatory markers are upregulated within 6 hours following injury. Immunohistochemistry shows activation of microglia and infiltration of CD45-positive leukocytes in the optic nerve and initiation of a gliotic response. The SI-TON model is capable of delivering a non-contact concussive injury to the optic nerve and induce TON in mice.

ONC-TON is induced by exposing the optic nerve of an adult rat and creating a small opening in the meninges of the nerve, as described in Solomon, A. et al., J. of Neurosci. Methods (1996) 70: 21-25, (incorporated herein by reference). The opening is created about 2-3 mm behind the eye globe. A glass dissector is introduced through the opening and is used to cut all the axons through the whole width of the nerve. Complete transfection of the optic nerve axons is achieved, while retaining the continuity of the meninges and avoiding damage to the nerve's vascular supply. Transection can be confirmed by transillumination showing a complete gap in the continuity of the nerve axons, and by both morphological and electrophysiological criteria. The opening created in the ‘meningeal tube’ can be used to inject substances that may be of benefit in recovery, rescue and/or regeneration of the injured axons. The model is particularly suitable for in vivo studies on nerve regeneration, and especially for screening of putative therapeutic agents.

The ocular blast model of TON is induced by use of specialized pressure chamber, as described in Hines-Beard, J. et al., Experimental Eye Res. (2012) 99: 63-70 (incorporated by reference herein). Briefly, the specialized pressure chamber comprises a pressurized air tank attached to a regulated paintball gun with a machined barrel; a chamber that protects the mouse from direct injury and recoil, while exposing the eye; and a secure platform that enables fine, controlled movement of the chamber in relation to the barrel. Mice are exposed to one of three blast pressures (23.6, 26.4, or 30.4 psi). Gross pathology, intraocular pressure, optical coherence tomography, and visual acuity can be assessed 0, 3, 7, 14, and 28 days after exposure. Contralateral eyes and non-blast exposed mice can be used as controls. Gross pathology of the TON in this model includes but is not limited to corneal edema, corneal abrasions, and optic nerve avulsion.

### In Vitro Models

In addition to the above-described animal models, in vitro models of optic neuropathies such as TON can comprise in vitro culture of retinal ganglion cells. Methods of deriving and/or culturing RGCs are known in the art and described, for example, in Xu, Z. et al., J Huazhong Univ Sci Technolog Med Sci. (2011) 31: 400-3; Hu, D. et al., J Glaucoma (1997) 6: 37-43; Tanaka, T. et al., Nature Scientific Reports (2015) 5: 8344 (each incorporated herein by reference).

### Methods of Assessing Optic Nerve Function and Treatment Efficacy

In some embodiments of the methods of the present technology, the subject's visual function and/or the efficacy of treatment is assessed by one or more of: best corrected visual acuity (BVCA), pattern electroretinography (PERG), electroretinogram (ERG), scotopic threshold response (STR), optical coherence tomography (OCT), visual evoked potential (VEP), pattern reversal VEP, and photopic negative response (PhNR). Standards for these techniques been published, for example, by the International Society for Clinical Electrophysiology of Vision (ISCEV) in Marmor, M. F. et al., Doc. Opthamol. (1995) 89: 199-210; Holder, G. E. et al., Doc. Opthamol. (2007) 114: 111-116; Marmor, M. F. et al., Doc. Opthamol. (2008) 118: 69-77; Odom, J. V. et al., Doc. Opthamol. (2009) 120: 111-19; and Hood, D. et al., Doc. Opthamol. (2012) 124: 1-13.

Visual acuity and best corrected visual acuity (BCVA) are used interchangeably to refer to the maximum resolution of the eye, as a function of the eye's the spatial resolution of the visual processing system. BCVA is tested by requiring a test subject to identify optotypes such as stylized letters, Landolt rings, symbols, standardized Cyrillic letters, or other patterns on a chart from a set viewing distance. Optotypes are represented with maximum contrast (e.g., as black symbols against a white background). The distance between the test subject's eyes and the testing chart is set so as to approximate “optical infinity” in the way the lens attempts to focus (far acuity), or at a defined reading distance (near acuity). In some embodiments, measurement can be performed by using an eye chart (e.g., charts of Ferdinand Monoyer), by optical instruments, and/or by computerized tests like the FrACT.

PERG is an established technique for the objective assessment of central retinal function. In some embodiments, PERG involves use of a reversing checkerboard to evoke small electrical potentials that largely arise from inner retina. The normal PERG, using techniques recommended by the International Society for Clinical Electrophysiology of Vision (ISCEV), is recorded using corneal electrodes that do not interfere with the optics of the eye. In some embodiments, PERG consists of a prominent positive component at approximately 50 ms and a larger negativity at approximately 95 ms. These components are known as P50 and N95 according to conventional neurophysiological practice whereby a component is identified by its polarity and approximate latency. In some embodiments, N95 is generated in relation to retinal ganglion cell function. Some of P50 generated more distally, but in some embodiments, up to 70% of P50 has origins in relation to spiking cell function. In some embodiments, the P50 component is “driven” by the macular photoreceptors and can thus be used as an index of macular function. A “steady state” waveform is obtained if a rapid (>3.5 Hz) stimulus rate is used; however, this does not allow measurement of individual components. In some embodiments, PERG is performed as described, e.g., in Holder, G. E. et al., Doc. Opthamol. (2007) 114: 111-116; and Holder, G. Progress in Retinal and Eye Research (2001) 20: 531-561 (each incorporated herein by reference).

In some embodiments, optic nerve dysfunction caused by traumatic injury manifests with electrophysiological abnormalities. In some embodiments, a subject having or suspected of having optic neuropathy, such as TON, exhibits PERG amplitudes that continuously decrease over time in the affected eye and or an increase in peak latency over time. Accordingly, in some embodiments, success of treatment of TON and/or improvement in a subject's visual function can be determined, for example, by detecting an improvement in one or more PERG measures such as latency and amplitude following injury. In some embodiments, the improvement is an increase in PERG amplitude and/or a reduction in latency delay.

The ERG is the mass response of the retina, usually to a diffuse short-duration flash delivered via a Ganzfeld bowl. It is recorded using corneal electrodes. The main components of the ERG are the negative going a-wave and the positive going b-wave. The a-wave, in response to a bright flash in a dark-adapted eye, largely reflects photoreceptor function, but there may be a contribution from postreceptoral structures, particularly with low stimulus luminance. The b-wave, which is of higher amplitude than the a-wave in normals, reflects postphototransduction activity. It is largely produced in relation to ON-(depolarising) bipolar cell function. The ISCEV Standard ERG (Marmor, M. F. et al., Doc. Opthamol. (1995) 89: 199-210) incorporates a rod-specific response to a dim light under scotopic conditions, and a standard; mixed rod-cone response to a bright white flash under dark adaptation. This latter response is dominated by rod function. In some embodiments, the ERG is a mass response and therefore may elicit a normal reading when dysfunction is confined to small retinal areas. In some embodiments, Photopic ERGs are recorded both to a single flash (with adequate photopic adaptation and a rod-suppressing background) and to a 30 Hz flicker stimulus; rods are unable to respond to a 30 Hz stimulus due to poor temporal resolution. In some embodiments, a photopic negative response (PhNR) assessment is performed measuring response to a brief flash a negative-going wave following the b-wave of the cone response. In some embodiments, a scotopic threshold response (STR) assessment is performed wherein a dim light evokes a small, corneal-negative wave in the ERG of a fully dark adapted human eye. In some embodiments, the ERG is performed as described, e.g., in Marmor, M. F. et al., Doc. Opthamol. (1995) 89: 199-210; Marmor, M. F. et al., Doc. Opthamol. (2009) 118: 69-77; Heckenlively, J. R. and Arden, G. B. (eds) (1991) Principles and Practice of Clinical Electrophysiology of Vision. Mosby Year Book, St. Louis; Fishman, G. A. et al. Opthamology Monograph 2, 2^{nd} Edition (2001) The Foundation of the American Academy of Ophthalmology, San Francisco (each incorporated herein by reference).

The visual-evoked cortical potential (VEP) is an important electrophysiological test in the investigation of suspected optic nerve disease. The stimulus for diagnostic VEP is usually a reversing black and white checkerboard or grating (PVEP), but an appearance stimulus (onset/offset) can also be used. Diffuse flash stimulation has a role, but the flash VEP (FVEP) is less sensitive to the effects of disease than the pattern VEP, and is highly variable across a population. However, due to its low interocular or interhemispheric asymmetry in a normal subject, the FVEP may detect interocular or interhemispheric asymmetry within an individual patient. In some embodiments, the VEP evoked by a pattern reversal stimulus consists of a prominent positive component at approximately 100 ms (P100) preceded and followed by negative components (N75 and N135). In some embodiments, analysis concentrates on the latency (to peak) and amplitude of the P100 component. In addition to the detection of anterior visual pathway dysfunction, chiasmal and retro-chiasmal dysfunction can be assessed by examination of the distribution of the VEP over the posterior regions of the scalp. In some embodiments, the VEP is performed as described, e.g., in Odom, J. V. et al., Doc. Opthamol. (2009) 120: 111-19; and Heckenlively, J. R. and Arden, G. B. (eds) (1991) Principles and Practice of Clinical Electrophysiology of Vision. Mosby Year Book, St. Louis (each incorporated herein by reference).

The pattern reversal VEP (prVEP) consists of a prominent positive component at approximately 100 ms (P100) preceded and followed by negative components (N75 and N135). Analysis concentrates on the implicit time (usually termed latency) and amplitude of P100. In addition to the detection of optic nerve dysfunction, chiasmal and retrochiasmal dysfunction can be assessed by examining the distribution of the VEP over the posterior scalp. Although a delayed P100 component often occurs in association with optic nerve disease, delays are also commonplace in macular dysfunction, and a delayed VEP should not be considered pathognomonic of optic nerve disease. An associated test of macular function, such as the pattern electroretinogram (PERG) or multifocal ERG (mfERG) allows an improved interpretation of an abnormal prVEP. In some embodiments, prVEP is performed as described in Kothari, R. et al. Int J. Opthamol. (2014) 7: 326-29 (incorporated herein by reference).

The multifocal ERG (mfERG) technique was developed to provide a topographic measure of retinal electrophysiological activity. The multifocal electroretinogram (mfERG) is a technique providing simultaneous assessment of local retinal areas using a pseudorandom binary sequence stimulation technique. In some embodiments, the stimulus consists of black and white hexagons covering approximately 50°. In some embodiments, ERG responses, typically 61 or 103, are recorded from the cone-driven retina under light-adapted conditions. The mfERG can therefore provide an index of central retinal function that extends the data provided by the PERG by giving additional spatial information, and, in some embodiments, layer localization within the retina. In some embodiments, the ERG is performed as described, e.g., in Hood, D. et al., Doc. Opthamol. (2012) 124: 1-13 (incorporated herein by reference).

Optical coherence tomography (OCT) refers to an imaging technique that uses coherent light to capture micrometer-resolution, two- and three-dimensional images from within optical scattering media (e.g., biological tissue). Optical coherence tomography is based on low-coherence interferometry, typically employing near-infrared light. The use of relatively long wavelength light allows it to penetrate into the scattering medium. OCT allows for an assessment of cellular organization, photoreceptor integrity, retinal microvasculature, retinal layer thickness, cup to disc ratio, and axonal thickness in the eye. The cup-to-disc ratio compares the diameter of the “cup” portion of the optic disc with the total diameter of the optic disc. Pathological cupping of the optic disc may occur in the presence of intraocular pressure.

In some embodiments, RGCs in a subject (i.e., in vivo) can be assessed by one or more of best corrected visual acuity (BCVA), pattern electroretinography (PERG), electroretinogram (ERG), scotopic threshold response (STR), optical coherence topography (OCT), visual evoked potential (VEP), pattern reversal VEP (prVEP), and photopic negative response (PhNR). Such methods can be used to detect RGC survival and/or increasing neurite outgrowth.

In some embodiments, RGCs can be assessed by any suitable method for assaying RGC damage, RGC survival, or neurite outgrowth known in the art. Neurite outgrowth refers to a neuronal morphological change in the projection(s) of a neuron that extend from the cell body (e.g., an axon or a dendrite). In some embodiments, detection of neurite outgrowth comprises detecting including increases in the number or frequency of neurites, and increases in neurite length or size. Nonlimiting examples of suitable methods to assess RGCs include, but are not limited to: use of an in vitro neurite outgrowth assay (available, for example, from Millipore Sigma, cat #NS220), use of a cell viability assay such as a tetrazolium reduction assay, a resazurin reduction assay, a dye exclusion assay such as a trypan blue assay, a cell proliferation assay such as cell quantitation, an apoptosis assay such as an Annexin V-based assay or a TUNEL assay, a protease viability marker assay, and an ATP assay.

In some embodiments, the pathophysiology of optic neuropathy in RGCs can be determined by immunohistochemistry (IHC) to stain the optic nerves for several markers including but not limited to one or more of the following proteins: activated microglia marker CD11b available from, for example, Abcam (cat #ab133357), leukocyte common antigen CD45 available from, for example, Abcam (cat #ab10558), platelet endothelial cell adhesion molecule (CD31) available from, for example, Abcam (cat #ab28364), tumor necrosis factor alpha (Tnf) available from, for example, Abcam (cat #ab6671), and the astrocyte marker GFAP available from, for example, Abcam (cat #ab7260). In some embodiments, damaged optic nerves exhibit one or more of: activation of microglia (CD11b-positive cells), infiltration of CD45-positive leukocytes in the optic nerve, accumulation of soluble Tnf protein, and positive staining of the astrocyte marker GFAP in both transverse and/or longitudinal optic nerve sections.

In some embodiments, the early pathophysiological mechanisms leading to RGC loss and visual function can be detected by gene expression analysis of selected markers. Nonlimiting examples of gene expression analysis include quantitative RT-PCR (qRT-PCR), RNA-seq, Northern blot, fluorescent in situ hybridization, serial analysis of gene expression (SAGE), Western blot, and microarray analysis on selected markers. In some embodiments, damaged RGCs show an upregulation of in expression of one or more pro-inflammatory moieties in the injured nerve including but not limited to Interleukin 1-beta (Il1b, RefSeq: NM_000576), Chemokine (C-C motif) ligand 2 (Ccl2, RefSeq: NM_002982), tumor necrosis factor-alpha (Tnf, RefSeq: NM_000594), and C-X-C motif chemokine 10 (Cxcl10, RefSeq: NM_001565). Accordingly, successful treatment of RGCs can be determined by detecting a decrease in expression of one or more of these genes relative to an untreated control with TON.

### Modes of Administration and Effective Dosages

Any method known to those in the art for contacting a cell, organ or tissue with an aromatic-cationic peptide of the present technology, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) may be employed. In some embodiments of the methods of the present technology, the aromatic-cationic peptide is D-Arg-2′,6′-Dmt-Lys-Phe-NH₂, or a pharmaceutically acceptable salt thereof. Suitable methods include in vitro, ex vivo, or in vivo methods. In vivo methods typically include the administration of an aromatic-cationic peptide, such as those described above, to a mammal, suitably a human. When used in vivo for therapy, the aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) are administered to the subject in effective amounts (i.e., amounts that have desired therapeutic effect). The dose and dosage regimen will depend upon the degree of the infection in the subject, the characteristics of the particular aromatic-cationic peptide used, e.g., its therapeutic index, the subject, and the subject's history.

The effective amount may be determined during pre-clinical trials and clinical trials by methods familiar to physicians and clinicians. An effective amount of a peptide useful in the methods may be administered to a mammal in need thereof by any of a number of well-known methods for administering pharmaceutical compounds. The peptide may be administered systemically or locally.

The peptide may be formulated as a pharmaceutically acceptable salt. The term “pharmaceutically acceptable salt” means a salt prepared from a base or an acid which is acceptable for administration to a patient, such as a mammal (e.g., salts having acceptable mammalian safety for a given dosage regime). However, it is understood that the salts are not required to be pharmaceutically acceptable salts, such as salts of intermediate compounds that are not intended for administration to a patient. Pharmaceutically acceptable salts can be derived from pharmaceutically acceptable inorganic or organic bases and from pharmaceutically acceptable inorganic or organic acids. In addition, when a peptide contains both a basic moiety, such as an amine, pyridine or imidazole, and an acidic moiety such as a carboxylic acid or tetrazole, zwitterions may be formed and are included within the term “salt” as used herein. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, trimethylamine (NEt₃), trimethylamine, tripropylamine, tromethamine and the like, such as where the salt includes the protonated form of the organic base (e.g., [HNEt₃]⁺). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like. In some embodiments, the pharmaceutically acceptable counterion is selected from the group consisting of acetate, benzoate, besylate, bromide, camphorsulfonate, chloride, chlortheophyllinate, citrate, ethandisulfonate, fumarate, glueptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride salt, a bis-hydrochloride salt, a tri-hydrochloride salt, a mono-tosylate salt, a bis-tosylate salt or a tri-tosylate salt. In some embodiments, the peptide that is formulated for administering to a subject is as a tri-HCl salt, a bis-HCl salt, or a mono-HCl salt.

The aromatic-cationic peptides described herein, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) can be incorporated into pharmaceutical compositions for administration, singly or in combination, to a subject for the treatment or prevention of a disorder described herein. In some embodiments of the methods of the present technology, the aromatic-cationic peptide is D-Arg-2′,6′-Dmt-Lys-Phe-NH₂, or a pharmaceutically acceptable salt thereof. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, intravitreal, inhalation, transdermal (topical), intraocular, ophthalmic, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The aromatic-cationic peptide compositions can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

For ophthalmic or intraocular formulations, any suitable mode of delivering the aromatic-cationic peptides described herein, such as 2′6′-Dmt-D-Arg-Phe-Lys-NH₂, Phe-D-Arg-Phe-Lys-NH₂, or D-Arg-2′6′-Dmt-Lys-Phe-NH₂, a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) or pharmaceutical compositions thereof to the eye or regions near the eye can be used. For ophthalmic formulations generally, see Mitra (ed.), Ophthalmic Drug Delivery Systems, Marcel Dekker, Inc., New York, N.Y. (1993) and also Havener, W. H., Ocular Pharmacology, C. V. Mosby Co., St. Louis (1983). Nonlimiting examples of formulations suitable for administration in or near the eye include, but are not limited to, ocular inserts, minitablets, and topical formulations such as eye drops, ointments, and in situ gels. In one embodiment, a contact lens is coated with the aromatic-cationic peptides described herein, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt). In some embodiments, a single dose comprises from between 0.1 ng to 5000 μg, 1 ng to 500 μg, or 10 ng to 100 μg of the aromatic-cationic peptides administered to the eye.

Eye drops comprise a sterile liquid formulation that can be administered directly to the eye. In some embodiments, eye drops comprising one or more aromatic-cationic peptides described herein, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) further comprise one or more preservatives. In some embodiments, the optimum pH for eye drops equals that of tear fluid and is about 7.4.

In situ gels are viscous liquids, showing the ability to undergo sol-to-gel transitions when influenced by external factors, such as appropriate pH, temperature, and the presence of electrolytes. This property causes slowing of drug drainage from the eyeball surface and increase of the active ingredient bioavailability. Polymers commonly used in in situ gel formulations include, but are not limited to, gellan gum, poloxamer, and cellulose acetate phthalate.

Ointments are semisolid dosage forms for external use such as topical use for the eye. In some embodiments, ointments comprise a solid or semisolid hydrocarbon base of melting or softening point close to human core temperature. In some embodiments, an ointment applied to the eye decomposes into small drops, which stay for a longer time period in conjunctival sac, thus increasing bioavailability.

Ocular inserts are solid or semisolid dosage forms without disadvantages of traditional ophthalmic drug forms. They are less susceptible to defense mechanisms like outflow through nasolacrimal duct, show the ability to stay in conjunctival sac for a longer period, and are more stable than conventional dosage forms. They also offer advantages such as accurate dosing of one or more aromatic-cationic peptides, slow release of one or more aromatic-cationic peptides with constant speed, and limiting of one or more aromatic-cationic peptides' systemic absorption. In some embodiments, an ocular insert comprises one or more aromatic-cationic peptides described herein, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) and one or more polymeric materials. The polymeric materials include, but are not limited to, methylcellulose and its derivatives (e.g., hydroxypropyl methylcellulose (HPMC)), ethylcellulose, polyvinylpyrrolidone (PVP K-90), polyvinyl alcohol, chitosan, carboxymethyl chitosan, gelatin, and various mixtures of the aforementioned polymers.

Minitablets are biodegradable, solid drug forms, that transit into gels after application to the conjunctival sac, thereby extending the period of contact between active ingredient and the eyeball surface, which in turn increases the active ingredient's bioavailability. The advantages of minitablets include easy application to conjunctival sac, resistance to defense mechanisms like tearing or outflow through nasolacrimal duct, longer contact with the cornea caused by presence of mucoadhesive polymers, and gradual release of the active ingredient from the formulation in the place of application due to the swelling of the outer carrier layers. Minitablets comprise one or more aromatic-cationic peptides described herein, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) and one or more polymers. Nonlimiting examples of polymers suitable for use in in a minitablet formulation include cellulose derivatives, like hydroxypropyl methylcellulose (HPMC), hydroxyethyl cellulose (HEC), sodium carboxymethyl cellulose, ethyl cellulose, acrylates (e.g., polyacrylic acid and its cross-linked forms), Carbopol or Carbomer, chitosan, and starch (e.g., drum-dried waxy maize starch). In some embodiments, minitablets further comprise one or more excipients. Nonlimiting examples of excipients include mannitol and magnesium stearate.

The ophthalmic or intraocular preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

In some embodiments, the viscosity of the ocular formulation comprising one or more aromatic-cationic peptides described herein, such as 22′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) is increased to improve contact with the cornea and bioavailability in the eye. Viscosity can be increased by the addition of hydrophilic polymers of high molecular weight which do not diffuse through biological membranes and which form three-dimensional networks in the water. Nonlimiting examples of such polymers include polyvinyl alcohol, poloxamers, hyaluronic acid, carbomers, and polysaccharides, cellulose derivatives, gellan gum, and xanthan gum.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic protein or peptide can be formulated in a carrier system. The carrier can be a colloidal system. The colloidal system can be a liposome, a phospholipid bilayer vehicle. In one embodiment, the therapeutic peptide is encapsulated in a liposome while maintaining peptide integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic peptide can be embedded in the polymer matrix, while maintaining protein integrity. The polymer may be natural, such as polypeptides, proteins or polysaccharides, or synthetic, such as poly a-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the aromatic-cationic peptides, sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of peptide ranges from 0.001-10,000 micrograms per kg body weight. In one embodiment, aromatic-cationic peptide concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of an aromatic-cationic peptide may be defined as a concentration of peptide at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

In some embodiments, a therapeutically effective amount of an aromatic-cationic peptide is administered prior to injury. In some embodiments, a therapeutically effective amount of an aromatic-cationic peptide is administered immediately following injury. In some embodiments, a therapeutically effective amount of an aromatic-cationic peptide is administered about 1 hour or less, about 2 hours or less, about 3 hours or less, about 4 hours or less, about 5 hours or less, about 6 hours or less, about 7 hours or less, about 8 hours or less, about 9 hours or less, about 10 hours or less, about 11 hours or less, about 12 hours or less, about 14 hours or less, about 16 hours or less, about 18 hours or less, about 20 hours or less, about 22 hours or less, or about 24 hours or less following the injury. In some embodiments, a therapeutically effective amount of an aromatic-cationic peptide is administered about 1 minute to about 6 hours, about 1 hour to about 12 hours, about 4 hours to about 24 hours, about 12 hours to about 36 hours, about 6 hours to about 48 hours, or about 24 to about 76 hours following injury. In some embodiments, a therapeutically effective amount of an aromatic-cationic peptide is administered daily for about 1 week or more, about 2 weeks or more, about 3 weeks or more, about 4 weeks or more, about 5 weeks or more, about 6 weeks or more, about 7 weeks or more, about 8 weeks or more, about 10 weeks or more, or about 12 weeks or more. In some embodiments, a therapeutically effective amount of an aromatic-cationic peptide is administered daily for about 1 week to about 12 weeks.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

### Combination Therapies

In some embodiments, the aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) may be combined with one or more additional therapies for the prevention or treatment of TON. In some embodiments of the methods of the present technology, the aromatic-cationic peptide is D-Arg-2′,6′-Dmt-Lys-Phe-NH₂, or a pharmaceutically acceptable salt thereof. In some embodiments, additional therapies include, but are not limited to, administration of steroids; surgical decompression of the optic canal; a combination of steroids and surgery; administration of a TNFα inhibitor, administration of a corticosteroid, administration of an IL-1R antagonist, administration of resveratrol, administration of a potassium channel blocker, administration of necrostatin-1, and reduction of the treated subject's core temperature.

In some embodiments, one or more TNFα inhibitors are administered separately, simultaneously, or sequentially with the aromatic-cationic peptide(s). In some embodiments, the TNFα inhibitor is selected from the group consisting of etanercept (Enbrel™), infliximab (Remicade™), adalimumab (Humira™), certolizumab (Cimzia™), and golimumab (Symponi™). In a particular embodiment, the TNFα inhibitor is etanercept. In some embodiments, the dose of TNFα inhibitor is about 0.5 mg/kg to about 2 mg/kg, about 5 mg/kg to about 100 mg/kg, about 10 mg/kg to about 75 mg/kg, or about 25 mg/kg to about 50 mg/kg. In some embodiments, the dose of TNFα inhibitor is 0.8 mg/kg, about 5 mg/kg, about 10 mg/kg, about 20 mg/kg, about 25 mg/kg, about 30 mg/kg, about 40 mg/kg, about 50 mg/kg, about 60 mg/kg, about 75 mg/kg, about 80 mg/kg, about 90 mg/kg, about 100 mg/kg, about 110 mg/kg, about 120 mg/kg, about 125 mg/kg, about 130 mg/kg, about 140 mg/kg, about 150 mg/kg, about 160 mg/kg, about 175 mg/kg, about 180 mg/kg, about 190 mg/kg, about 200 mg/kg, or more. In some embodiments, the TNFα inhibitor is administered twice per day, daily, every 48 hours, every 72 hours, twice per week, once per week, once every two weeks, once per month, once every 2 months, once every 3 months, or once every 6 months. In some embodiments, the dose of TNFα inhibitor is dependent upon the subject's weight and/or age.

In some embodiments, one or more IL-1R antagonists are administered separately, simultaneously, or sequentially with the aromatic-cationic peptide(s). In some embodiments, the IL-1R antagonist is encoded by the IL1RN gene (Entrez gene 3557, UniProt P18510). In some embodiments, the IL-1R antagonist comprises a fragment of the protein encoded by the IL1RN gene or a protein with at least 80% sequence similarity to the protein encoded by the IL1RN gene. In some embodiments, the IL-1R antagonist is anakinra (Kineret™). Anakinra differs from native human IL-1Ra in that it has the addition of a single methionine residue at its amino terminus. In some embodiments, the IL-1R antagonist is recombinant. In some embodiments, the dose of IL-1R antagonist is about 0.5 mg/kg to about 2 mg/kg, about 1 mg/kg to about 2 mg/kg, about 0.5 mg/kg to about 5 mg/kg, about 5 mg/kg to about 100 mg/kg, about 10 mg/kg to about 75 mg/kg, or about 25 mg/kg to about 50 mg/kg. In some embodiments, the dose of IL-1R antagonist is 0.8 mg/kg, about 5 mg/kg, about 10 mg/kg, about 20 mg/kg, about 25 mg/kg, about 30 mg/kg, about 40 mg/kg, about 50 mg/kg, about 60 mg/kg, about 75 mg/kg, about 80 mg/kg, about 90 mg/kg, about 100 mg/kg, about 110 mg/kg, about 120 mg/kg, about 125 mg/kg, about 130 mg/kg, about 140 mg/kg, about 150 mg/kg, about 160 mg/kg, about 175 mg/kg, about 180 mg/kg, about 190 mg/kg, about 200 mg/kg, or more. In some embodiments, the IL-1R antagonist is administered twice per day, daily, every 48 hours, every 72 hours, twice per week, once per week, once every two weeks, once per month, once every 2 months, once every 3 months, or once every 6 months. In some embodiments, the dose of IL-1R antagonist is dependent upon the subject's weight and/or age.

In some embodiments, resveratrol is administered separately, simultaneously, or sequentially with the aromatic-cationic peptide(s). Resveratrol (3,5,4′-trihydroxy-trans-stilbene) is a stilbenoid, a type of natural phenol, and a phytoalexin produced by several plants in response to injury or, when the plant is under attack by pathogens such as bacteria or fungi. Sources of resveratrol in food include, but are not limited to, the skin of grapes, blueberries, raspberries, mulberries. In some embodiments, resveratrol is selected from the group consisting of dihydro-resveratrol, epsilon-viniferin, pallidol, quadrangularin A, trans-diptoindonesin B, hopeaphenol, oxyresveratrol, piceatannol, piceid, pterostilbene, and 4′Methoxy-(E)-resveratrol 3-O-rutinoside. In some embodiments, the dose of resveratrol is about 0.5 mg/kg to about 2 mg/kg, about 1 mg/kg to about 2 mg/kg, about 0.5 mg/kg to about 5 mg/kg, about 5 mg/kg to about 100 mg/kg, about 10 mg/kg to about 75 mg/kg, or about 25 mg/kg to about 50 mg/kg. In some embodiments, the dose of resveratrol is 0.8 mg/kg, about 5 mg/kg, about 10 mg/kg, about 20 mg/kg, about 25 mg/kg, about 30 mg/kg, about 40 mg/kg, about 50 mg/kg, about 60 mg/kg, about 75 mg/kg, about 80 mg/kg, about 90 mg/kg, about 100 mg/kg, about 110 mg/kg, about 120 mg/kg, about 125 mg/kg, about 130 mg/kg, about 140 mg/kg, about 150 mg/kg, about 160 mg/kg, about 175 mg/kg, about 180 mg/kg, about 190 mg/kg, about 200 mg/kg, or more. In some embodiments, the resveratrol is administered twice per day, daily, every 48 hours, every 72 hours, twice per week, once per week, once every two weeks, once per month, once every 2 months, once every 3 months, or once every 6 months. In some embodiments, the dose of resveratrol is dependent upon the subject's weight and/or age.

In some embodiments, one or more necrostatin-1 agents are administered separately, simultaneously, or sequentially with the aromatic-cationic peptide(s). In some embodiments, the necrostatin-1 is a compound of the formula 5-((1H-indol-3-yl)methyl)-3-methyl-2-thioxoimidazolidin-4-one. In some embodiments, the necrostatin-1 agent is an analog of necrostatin-1 including but not limited to Nec-1 inactive (Nec-1i) having the formula (5-((1H-indol-3-yl)methyl)-2-thioxoimidazolidin-4-one), Nec-1 stable (Nec-1s) having the formula 7-Cl—O-Nec-1 (5-((7-chloro-1H-indol-3-yl)methyl)-3-methylimidazolidine-2,4-dione), or methyl-thiohydantoin-tryptophan, an inhibitor of the immunomodulatory enzyme indoleamine 2,3-dioxygenase (IDO). In some embodiments, the dose of necrostatin-1 agent is about 0.5 mg/kg to about 2 mg/kg, about 1 mg/kg to about 2 mg/kg, about 0.5 mg/kg to about 5 mg/kg, about 5 mg/kg to about 100 mg/kg, about 10 mg/kg to about 75 mg/kg, or about 25 mg/kg to about 50 mg/kg. In some embodiments, the dose of necrostatin-1 is 0.8 mg/kg, about 5 mg/kg, about 10 mg/kg, about 20 mg/kg, about 25 mg/kg, about 30 mg/kg, about 40 mg/kg, about 50 mg/kg, about 60 mg/kg, about 75 mg/kg, about 80 mg/kg, about 90 mg/kg, about 100 mg/kg, about 110 mg/kg, about 120 mg/kg, about 125 mg/kg, about 130 mg/kg, about 140 mg/kg, about 150 mg/kg, about 160 mg/kg, about 175 mg/kg, about 180 mg/kg, about 190 mg/kg, about 200 mg/kg, or more. In some embodiments, the necrostatin-1 agent is administered twice per day, daily, every 48 hours, every 72 hours, twice per week, once per week, once every two weeks, once per month, once every 2 months, once every 3 months, or once every 6 months. In some embodiments, the dose of necrostatin-1 is dependent upon the subject's weight and/or age.

In some embodiments, one or more potassium channel blocker agents are administered separately, simultaneously, or sequentially with the aromatic-cationic peptide(s). In some embodiments, the potassium channel blocker is 4-aminopyridine (4-AP). In some embodiments, the dose of potassium channel blocker agent is about 0.5 mg/kg to about 2 mg/kg, about 1 mg/kg to about 2 mg/kg, about 0.5 mg/kg to about 5 mg/kg, about 5 mg/kg to about 100 mg/kg, about 10 mg/kg to about 75 mg/kg, or about 25 mg/kg to about 50 mg/kg. In some embodiments, the dose of potassium channel blocker is 0.8 mg/kg, about 5 mg/kg, about 10 mg/kg, about 20 mg/kg, about 25 mg/kg, about 30 mg/kg, about 40 mg/kg, about 50 mg/kg, about 60 mg/kg, about 75 mg/kg, about 80 mg/kg, about 90 mg/kg, about 100 mg/kg, about 110 mg/kg, about 120 mg/kg, about 125 mg/kg, about 130 mg/kg, about 140 mg/kg, about 150 mg/kg, about 160 mg/kg, about 175 mg/kg, about 180 mg/kg, about 190 mg/kg, about 200 mg/kg, or more. In some embodiments, the potassium channel blocker agent is administered twice per day, daily, every 48 hours, every 72 hours, twice per week, once per week, once every two weeks, once per month, once every 2 months, once every 3 months, or once every 6 months. In some embodiments, the dose of potassium channel blocker is dependent upon the subject's weight and/or age.

In some embodiments, a therapeutic cooling treatment comprising reducing the subject's temperature is administered or performed separately, simultaneously, or sequentially with administration of the aromatic-cationic peptide(s). In some embodiments, the temperature of the subject is reduced by about 2%, about 3%, about 4%, about 5%, about 6%, about 7%, about 8%, about 9%, about 10%, about 15%, about 20%, about 25%, about 30%, about 35%, about 40%, about 45%, or about 50% relative to the subject's temperature prior to performing temperature reduction. In particular embodiments, the subject's temperature is reduced about 2% to about 6% or about 3% to about 10%. In a non-limiting example, a subject's temperature is reduced from about 37° C. to between about 32° C. to about 34° C. In some embodiments, the temperature is the core temperature of the subject. In some embodiments, the subject's temperature is reduced via use of one or more of the following: cooling blankets, ice, ice packs, cooling pads, ice water, and chilled fluids administered through an IV (intravenous) line into the bloodstream. In some embodiments, the reduced temperature is maintained for about 2 hours, about 4 hours, about 6 hours, about 8 hours, about 10 hours, about 12 hours, about 14 hours, about 16 hours, about 18 hours, about 20 hours, about 22 hours, about 24 hours, about 30 hours, about 36 hours, about 42 hours, about 48 hours, or about 72 hours. In a particular embodiment, the subject's temperature is reduced for about 24 hours. In some embodiments, the therapeutic cooling treatment is initiated within 4 to 6 hours after injury. In some embodiments, a sedative and/or paralytic agent is co-administered with the therapeutic cooling treatment to prevent the subject from shivering and/or moving. In some embodiments, the sedative and/or paralytic agent is one or more of fentanyl, propofol, midazolam, and vecuronium. In some embodiments, hypothermia is induced in the subject. In some embodiments, hypothermia refers to a subject with a core temperature below 35° C. Exemplary methods for therapeutic cooling treatments are described, for example, in Samaniego, E. et al., Neurocrit Care. (2011) August; 15(1): 113-119, incorporated herein by reference.

In some embodiments, one or more steroids are administered separately, simultaneously, or sequentially with the aromatic-cationic peptide(s). In some embodiments, the one or more steroids comprise corticosteroids. Nonlimiting examples of suitable corticosteroids include methylprednisolone, prednisone, dexamethasone, hydrocortisone, and prednisolone. In some embodiments, the dose of steroid is about 0.5 mg/kg to about 2 mg/kg, about 1 mg/kg to about 2 mg/kg, about 0.5 mg/kg to about 5 mg/kg, about 5 mg/kg to about 100 mg/kg, about 10 mg/kg to about 75 mg/kg, or about 25 mg/kg to about 50 mg/kg. In some embodiments, the dose of steroid is 0.8 mg/kg, about 5 mg/kg, about 10 mg/kg, about 20 mg/kg, about 25 mg/kg, about 30 mg/kg, about 40 mg/kg, about 50 mg/kg, about 60 mg/kg, about 75 mg/kg, about 80 mg/kg, about 90 mg/kg, about 100 mg/kg, about 110 mg/kg, about 120 mg/kg, about 125 mg/kg, about 130 mg/kg, about 140 mg/kg, about 150 mg/kg, about 160 mg/kg, about 175 mg/kg, about 180 mg/kg, about 190 mg/kg, about 200 mg/kg, or more. In some embodiments, the steroid is administered twice per day, daily, every 48 hours, every 72 hours, twice per week, once per week, once every two weeks, once per month, once every 2 months, once every 3 months, or once every 6 months. In particular embodiments, a low dose of less than about 100 mg, a moderate dose of about 100 mg to about 499 mg, a high dose of about 500 mg to about 1999 mg, a very high dose of about 2000 to about 5399 mg, or a megadose of greater than about 5400 mg of steroids is administered to the subject.

In some embodiments, one or more antioxidants are administered separately, simultaneously, or sequentially with the aromatic-cationic peptide(s). The use of antioxidants has been shown to benefit patients with ophthalmic disorders. See, e.g., Arch. Ophthalmol., 119: 1417-36 (2001); Sparrow, et al., J. Biol. Chem., 278:18207-13 (2003). Examples of suitable antioxidants that could be used in combination with at least one aromatic-cationic peptide include vitamin C, vitamin E, beta-carotene and other carotenoids, coenzyme Q, 4-hydroxy-2,2,6,6-tetramethylpiperidine-N-oxyl (also known as Tempol), lutein, butylated hydroxytoluene, resveratrol, a trolox analogue (PNU-83836-E), and bilberry extract.

In some embodiments, one or more minerals are administered separately, simultaneously, or sequentially with the aromatic-cationic peptide(s). The use of certain minerals has also been shown to benefit patients with ophthalmic disorders. See, e.g., Arch. Ophthalmol., 119: 1417-36 (2001). Examples of suitable minerals that could be used in combination with at least one aromatic-cationic peptide include copper-containing minerals, such as cupric oxide; zinc-containing minerals, such as zinc oxide; and selenium-containing compounds.

In some embodiments, one or more negatively charged phospholipids are administered separately, simultaneously, or sequentially with the aromatic-cationic peptide(s). The use of certain negatively-charged phospholipids has also been shown to benefit patients with ophthalmic disorders. See, e.g., Shaban & Richter, Biol. Chem., 383:537-45 (2002); Shaban, et al., Exp. Eye Res., 75:99-108 (2002). Examples of suitable negatively charged phospholipids that could be used in combination with at least one aromatic-cationic peptide include cardiolipin and phosphatidylglycerol. Positively-charged and/or neutral phospholipids may also provide benefit for patients with ophthalmic disorders when used in combination with aromatic-cationic peptides.

In some embodiments, one or more carotenoids are administered separately, simultaneously, or sequentially with the aromatic-cationic peptide(s). The use of certain carotenoids has been correlated with the maintenance of photoprotection necessary in photoreceptor cells. Carotenoids are naturally-occurring yellow to red pigments of the terpenoid group that can be found in plants, algae, bacteria, and certain animals, such as birds and shellfish. Carotenoids are a large class of molecules in which more than 600 naturally occurring carotenoids have been identified. Carotenoids include hydrocarbons (carotenes) and their oxygenated, alcoholic derivatives (xanthophylls). They include actinioerythrol, astaxanthin, canthaxanthin, capsanthin, capsorubin, β-8′-apo-carotenal (apo-carotenal), β-12′-apo-carotenal, α-carotene, β-carotene, “carotene” (a mixture of α- and β-carotenes), γ-carotenes, β-cyrptoxanthin, lutein, lycopene, violerythrin, zeaxanthin, and esters of hydroxyl- or carboxyl-containing members thereof. Many of the carotenoids occur in nature as cis- and trans-isomeric forms, while synthetic compounds are frequently racemic mixtures.

In humans, the retina selectively accumulates mainly two carotenoids: zeaxanthin and lutein. These two carotenoids are thought to aid in protecting the retina because they are powerful antioxidants and absorb blue light. Studies with quails establish that groups raised on carotenoid-deficient diets had retinas with low concentrations of zeaxanthin and suffered severe light damage, as evidenced by a very high number of apoptotic photoreceptor cells, while the group with high zeaxanthin concentrations had minimal damage. Examples of suitable carotenoids for in combination with at least one aromatic-cationic peptide include lutein and zeaxanthin, as well as any of the aforementioned carotenoids.

In some embodiments, one or more nitric oxide inducers are administered separately, simultaneously, or sequentially with the aromatic-cationic peptide(s). Suitable nitric oxide inducers include compounds that stimulate endogenous NO or elevate levels of endogenous endothelium-derived relaxing factor (EDRF) in vivo or are substrates for nitric oxide synthase. Such compounds include, for example, L-arginine, L-homoarginine, and N-hydroxy-L-arginine, including their nitrosated and nitrosylated analogs (e.g., nitrosated L-arginine, nitrosylated L-arginine, nitrosated N-hydroxy-L-arginine, nitrosylated N-hydroxy-L-arginine, nitrosated L-homoarginine and nitrosylated L-homoarginine), precursors of L-arginine and/or physiologically acceptable salts thereof, including, for example, citrulline, ornithine, glutamine, lysine, polypeptides comprising at least one of these amino acids, inhibitors of the enzyme arginase (e.g., N-hydroxy-L-arginine and 2(S)-amino-6-boronohexanoic acid) and the substrates for nitric oxide synthase, cytokines, adenosine, bradykinin, calreticulin, bisacodyl, and phenolphthalein. EDRF is a vascular relaxing factor secreted by the endothelium, and has been identified as nitric oxide or a closely related derivative thereof (Palmer, et al., Nature, 327:524-526 (1987); Ignarro, et al., Proc. Natl. Acad. Sci. USA, 84:9265-9269 (1987)).

In some embodiments, one or more statins are administered separately, simultaneously, or sequentially with the aromatic-cationic peptide(s). Statins serve as lipid-lowering agents and/or suitable nitric oxide inducers. In addition, a relationship has been demonstrated between statin use and delayed onset or development of certain ophthalmic disorders. G. McGwin, et al., British Journal of Ophthalmology, 87:1121-25 (2003). Suitable statins include, by way of example only, rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium (which is the hemicalcium salt of atorvastatin), and dihydrocompactin.

In some embodiments, one or more anti-inflammatory agents are administered separately, simultaneously, or sequentially with the aromatic-cationic peptide(s). Suitable anti-inflammatory agents with which the aromatic-cationic peptides may be used include, by way of example only, aspirin and other salicylates, cromolyn, nedocromil, theophylline, zileuton, zafirlukast, montelukast, pranlukast, indomethacin, and lipoxygenase inhibitors; non-steroidal antiinflammatory drugs (NSAIDs) (such as ibuprofen and naproxin); prednisone, dexamethasone, cyclooxygenase inhibitors (i.e., COX-1 and/or COX-2 inhibitors such as Naproxen™, or Celebrex™); statins (by way of example only, rosuvastatin, pitivastatin, simvastatin, pravastatin, cerivastatin, mevastatin, velostatin, fluvastatin, compactin, lovastatin, dalvastatin, fluindostatin, atorvastatin, atorvastatin calcium (which is the hemicalcium salt of atorvastatin), and dihydrocompactin); and disassociated steroids.

In some embodiments, one or more antiangiogenic or anti-VEGF agents are administered separately, simultaneously, or sequentially with the aromatic-cationic peptide(s). The use of antiangiogenic or anti-VEGF drugs has also been shown to provide benefit for patients with ophthalmic disorders. Examples of suitable antiangiogenic or anti-VEGF drugs that could be used in combination with at least one aromatic-cationic peptide include Rhufab V2 (Lucentis™), Tryptophanyl-tRNA synthetase (TrpRS), Eye001 (Anti-VEGF Pegylated Aptamer), squalamine, Retaane™ 15 mg (anecortave acetate for depot suspension; Alcon, Inc.), Combretastatin A4 Prodrug (CA4P), Macugen™, Mifeprex™ (mifepristone—ru486), subtenon triamcinolone acetonide, intravitreal crystalline triamcinolone acetonide, Prinomastat (AG3340—synthetic matrix metalloproteinase inhibitor, Pfizer), fluocinolone acetonide (including fluocinolone intraocular implant, Bausch & Lomb/Control Delivery Systems), VEGFR inhibitors (Sugen), and VEGF-Trap (Regeneron/Aventis).

In some embodiments, other pharmaceutical therapies that have been used to relieve visual impairment can be used in combination with at least one aromatic-cationic peptide. Such treatments include, but are not limited to, agents such as Visudyne™ with use of a non-thermal laser, PKC 412, Endovion (NeuroSearch A/S), neurotrophic factors, including by way of example Glial Derived Neurotrophic Factor and Ciliary Neurotrophic Factor, diatazem, dorzolamide, Phototrop, 9-cis-retinal, eye medication (including Echo Therapy) including phospholine iodide or echothiophate or carbonic anhydrase inhibitors, AE-941 (AEterna Laboratories, Inc.), Sirna-027 (Sirna Therapeutics, Inc.), pegaptanib (NeXstar Pharmaceuticals/Gilead Sciences), neurotrophins (including, by way of example only, NT-4/5, Genentech), Cand5 (Acuity Pharmaceuticals), ranibizumab (Genentech), INS-37217 (Inspire Pharmaceuticals), integrin antagonists (including those from Jerini AG and Abbott Laboratories), EG-3306 (Ark Therapeutics Ltd.), BDM-E (BioDiem Ltd.), thalidomide (as used, for example, by EntreMed, Inc.), cardiotrophin-1 (Genentech), 2-methoxyestradiol (Allergan/Oculex), DL-8234 (Toray Industries), NTC-200 (Neurotech), tetrathiomolybdate (University of Michigan), LYN-002 (Lynkeus Biotech), microalgal compound (Aquasearch/Albany, Mera Pharmaceuticals), D-9120 (Celltech Group p 1c), ATX-S10 (Hamamatsu Photonics), TGF-beta 2 (Genzyme/Celtrix), tyrosine kinase inhibitors (Allergan, SUGEN, Pfizer), NX-278-L (NeXstar Pharmaceuticals/Gilead Sciences), Opt-24 (OPTIS France SA), retinal cell ganglion neuroprotectants (Cogent Neurosciences), N-nitropyrazole derivatives (Texas A&M University System), KP-102 (Krenitsky Pharmaceuticals), and cyclosporine A.

In some embodiments, an aromatic-cationic peptide may also be used in combination with procedures that may provide additional or synergistic benefits to the patient. Procedures known, proposed or considered to relieve visual impairment include, but are not limited to, “limited retinal translocation,” photodynamic therapy (including, by way of example only, receptor-targeted PDT, Bristol-Myers Squibb, Co.; porfimer sodium for injection with PDT; verteporfin, QLT Inc.; rostaporfin with PDT, Miravent Medical Technologies; talaporfin sodium with PDT, Nippon Petroleum; motexafin lutetium, Pharmacyclics, Inc.), antisense oligonucleotides (including, by way of example, products tested by Novagali Pharma SA and ISIS-13650, Isis Pharmaceuticals), laser photocoagulation, drusen lasering, macular hole surgery, macular translocation surgery, implantable miniature telescopes, Phi-Motion Angiography (also known as Micro-Laser Therapy and Feeder Vessel Treatment), Proton Beam Therapy, microstimulation therapy, Retinal Detachment and Vitreous Surgery, Scleral Buckle, Submacular Surgery, Transpupillary Thermotherapy, Photosystem I therapy, use of RNA interference (RNAi), extracorporeal rheopheresis (also known as membrane differential filtration and Rheotherapy), microchip implantation, stem cell therapy, gene replacement therapy, ribozyme gene therapy (including gene therapy for hypoxia response element, Oxford Biomedica; Lentipak, Genetix; PDEF gene therapy, GenVec), photoreceptor/retinal cells transplantation (including transplantable retinal epithelial cells, Diacrin, Inc.; retinal cell transplant, Cell Genesys, Inc.), and acupuncture.

In one embodiment, an additional therapeutic agent is administered to a subject in combination with at least one aromatic-cationic peptide, such that a synergistic therapeutic effect is produced. For example, administration of at least one aromatic-cationic peptide with one or more additional therapeutic agents for the prevention or treatment of TON will have greater than additive effects in the prevention or treatment of the disease. Therefore, lower doses of one or more of any individual therapeutic agent may be used in treating or preventing TON resulting in increased therapeutic efficacy and decreased side-effects. In some embodiments, at least one aromatic-cationic peptide is administered in combination with one or more a TNFα inhibitor, a corticosteroid, an IL-1R antagonist, resveratrol, a potassium channel blocker, or necrostatin-1, such that a synergistic effect in the prevention or treatment of optic neuropathy results.

In some embodiments, multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### EXAMPLES

The present technology is further illustrated by the following examples, which should not be construed as limiting in any way. For each of the examples below, any aromatic-cationic peptide described herein could be used. By way of example, but not by limitation, the aromatic-cationic peptide used in the example below could be 2′6′-Dmt-D-Arg-Phe-Lys-NH₂, Phe-D-Arg-Phe-Lys-NH₂, or D-Arg-2′6′-Dmt-Lys-Phe-NH₂ or any one or more of the peptides shown in Tables A, 6, 7, and/or 8.

### Example 1—Use of Aromatic-Cationic Peptides in the Treatment of TON

This example demonstrates the use of aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) in the treatment of TON.

### Methods

Subjects suspected of having or diagnosed as having TON receive daily administrations of 1 mg/kg body weight of aromatic-cationic peptide, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) alone or in combination with one or more additional therapeutic agents for the treatment or prevention of TON. Peptides and/or additional therapeutic agents are administered orally, topically, systemically, intravenously, subcutaneously, intravitreally, intraperitoneally, or intramuscularly according to methods known in the art. Subjects will be evaluated weekly for the presence and/or severity of signs and symptoms associated with TON including, but not limited to, e.g., vision loss, blurred vision, RGC damage, scotoma, decreased color sensation, uveitis, optic neuritis, eye pain, optic nerve avulsion, optic nerve transection, optic nerve sheath hemorrhage, orbital hemorrhage, choroidal rupture, and commotio retinae. Treatments are maintained until such a time as one or more signs or symptoms of TON are ameliorated or eliminated.

### Results

It is predicted that subjects suspected of having or diagnosed as having TON and receiving therapeutically effective amounts of aromatic-cationic peptide, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) will display reduced severity or elimination of one or more symptoms associated with TON. It is further expected that administration of 2′6′-Dmt-D-Arg-Phe-Lys-NH₂, Phe-D-Arg-Phe-Lys-NH₂, or D-Arg-2′6′-Dmt-Lys-Phe-NH₂ in combination with one or more additional therapeutic agents will have synergistic effects in this regard compared to that observed in subjects treated with the aromatic-cationic peptides or the additional therapeutic agents alone.

These results will show that aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) are useful in the treatment of TON. These results will show that aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an acetate salt, a tartrate salt, a trifluoroacetate salt, a chloride salt, a tris-HCl salt, a bis-HCl salt, a mono-HCl salt, or a tosylate salt) are useful in ameliorating one or more of the following symptoms: vision loss, blurred vision, RGC damage, scotoma, decreased color sensation, uveitis, optic neuritis, eye pain, optic nerve avulsion, optic nerve transection, optic nerve sheath hemorrhage, orbital hemorrhage, choroidal rupture, and commotio retinae. Accordingly, the peptides are useful in methods to treat subjects in need thereof for the treatment of TON.

### Example 2—Use of Aromatic-Cationic Peptides in the Treatment of TON in Animal Models

This example demonstrates the in vivo efficacy of aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) in treating TON in animal models of the disease.

### Methods

Two models for TON were utilized in this example: a novel sonication-induced TON (SI-TON) that closely recapitulates the clinical manifestations of indirect TON, and optic nerve crush-induced TON (ONC-TON) which reflects a more severe form of TON often resulting in axotomized nerve fibers and ruptured nervous vasculature. SI-TON is induced by a microtip probe sonifier placed on the supraorbital ridge directly above the entrance of the optic nerve into the bony canal, as described in Tao, W. et al., Scientific Reports (2017) 7: 11779. An ultrasonic pulse is then delivered to the optic nerve. ONC-TON is induced by exposing the optic nerve of an adult rat and creating a small opening in the meninges of the nerve, as described in Solomon, A. et al., J. of Neurosci. Methods (1996) 70: 21-25.

A baseline recording of visual function by pattern electroretinogram (PERG) and imaging of the optic disc was performed prior to inducing SI-TON or ONC-TON. Following TON induction, half of the animals then immediately received either a bolus subcutaneous injection of saline as a control or D-Arg-2′6′-Dmt-Lys-Phe-NH₂, followed by daily injections of their specific drug formulation for a period of 3 days. A subset of animals was allowed to progress to a neuropathic state for 3 days prior to receiving a bolus subcutaneous injection of saline or D-Arg-2′6′-Dmt-Lys-Phe-NH₂, followed by daily injections of their respective drug formulation for 2 consecutive days. On day 7 post-TON induction, animals underwent visual function testing through PERG, and retinal brightfield imaging of optic disc. In some cases, animals were euthanized at day 7 post-TON after functional testing and imaging. Euthanized animals were evaluated for RGC dropout by flat mount. Optic nerves were dissected posteriorly through the bony optic canal, sectioned with a VT-1000s vibratome, and processed for immunohistochemical analysis of inflammatory and gliotic markers.

To test if a combinatorial strategy results in improved visual outcomes, a therapeutic agent, such as a TNF-alpha inhibitor (e.g., etanercept), D-Arg-2′6′-Dmt-Lys-Phe-NH₂, or a combination of these pharmaceutical agents was assessed. A baseline recording of visual function by pattern electroretinogram (PERG) and imaging of the optic disc was performed prior to inducing TON in the animals. Half of the animals immediately received either a bolus subcutaneous injection of etanercept+D-Arg-2′6′-Dmt-Lys-Phe-NH₂, followed by daily injections of the drug formulation for a period of 3 days. A subset of animals was allowed to progress to a neuropathic state for 3 days and then will received a bolus subcutaneous injection of etanercept+D-Arg-2′6′-Dmt-Lys-Phe-NH₂, followed by daily injections of the drug formulation for 2 consecutive days. On day 7 post-TON, animals underwent visual function testing through PERG and retinal brightfield imaging of optic disc. Euthanized animals were evaluated for RGC dropout by flat mount. Optic nerves were dissected posteriorly through the bony optic canal, sectioned with a VT-1000s vibratome, and processed for immunohistochemical analysis of inflammatory and gliotic markers.

### Results

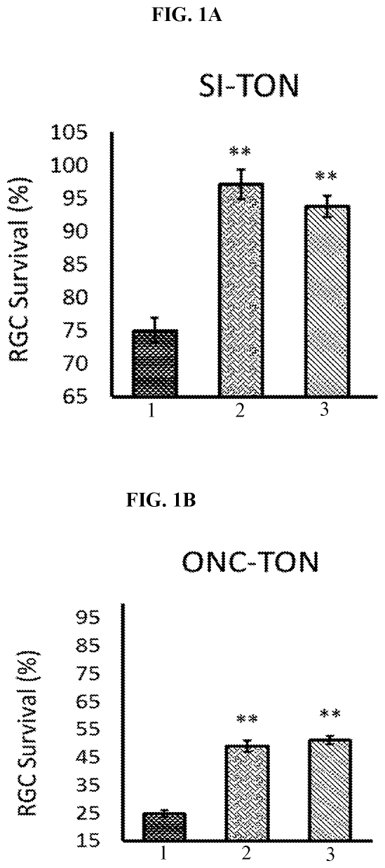

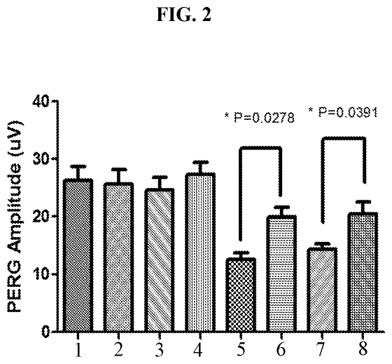

D-Arg-2′6′-Dmt-Lys-Phe-NH₂ promotes RGC survival in TON. The early use of D-Arg-2′6′-Dmt-Lys-Phe-NH₂ following TON induction results in significant survival of RGCs in both SI-TON and ONC-TON models (93.77±1.56% [p=0.00019], and 51.13±1.47% [p=1.98e-05], respectively). Early use (2 mg/kg SQ for 7 days) of D-Arg-2′6′-Dmt-Lys-Phe-NH₂ resulted in comparable RGC survival effects to the use of TNF-alpha inhibitor, etanercept, in both TON models (93.77% vs 97.08% in SI-TON, and 51.13% vs 48.94% in ONC-TON) (FIGS. 1A and 1B).

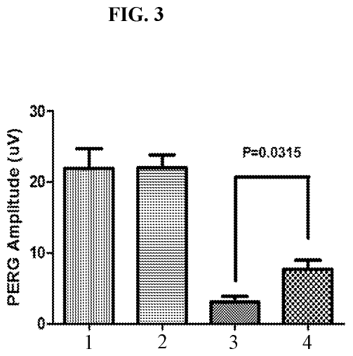

Early use of D-Arg-2′6′-Dmt-Lys-Phe-NH₂ significantly improves visual outcomes in SI-TON. Treatment with D-Arg-2′6′-Dmt-Lys-Phe-NH₂ immediately following trauma to the optic nerve resulted in improved visual outcomes that remained significantly higher than controls even after discontinuation of the drug, as evidenced by PERG recordings at 1 week and 2 weeks post-injury. While functional recovery was not established to the level of naïve, or contralateral (uninjured) eye, the improvement in visual function was significant over saline-treated control injured eyes at 1 week (p=0.0278), and 2 weeks (p=0.0391) (FIG. 2).

Early use of D-Arg-2′6′-Dmt-Lys-Phe-NH₂ significantly improves visual outcomes in ONC-TON. Treatment with D-Arg-2′6′-Dmt-Lys-Phe-NH₂ immediately following crush of the optic nerve resulted in improved visual outcomes that remained significantly higher than controls even after discontinuation of the drug, as evidenced by PERG recordings at 1 week post-injury. While functional recovery was still significantly lower than in naïve, or contralateral (uninjured) eyes in this severe traumatic model, the improvement in visual function was significant over saline-treated control injured eyes at 1 week (p=0.0315) post-crush (FIG. 3).

As demonstrated herein, use of D-Arg-2′6′-Dmt-Lys-Phe-NH₂ immediately following traumatic injury to the optic nerve confers a significant survival and functional benefit for preserving vision, comparable to that of the use of TNF-alpha inhibitor etanercept. Surprisingly, D-Arg-2′6′-Dmt-Lys-Phe-NH₂ use in the ONC-TON model results in a survival of 51.13% of RGCs (FIG. 1B), which is a remarkable outcome in such a severe trauma model, and far better than most reports using neurotrophic agents even prior to injury. Accordingly, these results demonstrate that the compositions of the present technology are useful in methods for treating or preventing traumatic optic neuropathy (TON) in a subject in need thereof.

### Example 3—Use of Aromatic-Cationic Peptides in the Prevention of TON in an Animal Model

This example demonstrates the in vivo efficacy of aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) in preventing (i.e., delaying) the onset of symptoms of TON, in a mouse model.

### Methods

2′6′-Dmt-D-Arg-Phe-Lys-NH₂, Phe-D-Arg-Phe-Lys-NH₂, or D-Arg-2′6′-Dmt-Lys-Phe-NH₂ is formulated in water and administered once daily by subcutaneous bolus injection at either 1 or 5 mg/kg starting from 8 weeks of age. Control mice will be untreated or treated with a control peptide. Aromatic-cationic peptides treated mice will also be compared to treatment with other therapeutic agents such as etanercept.

Methods of inducing SI-TON or ONC-TON are performed on the animals 4 hours, 8 hours, 12 hours, 18 hours, 24 hours, 48 hours, 3 days, 4 days, 5 days, 6 days, 1 week, 2 weeks, 3 weeks, or 4 weeks following the initial dose of peptide. SI-TON is performed by a microtip probe sonifier placed on the supraorbital ridge directly above the entrance of the optic nerve into the bony canal, as described in Tao, W. et al., Scientific Reports (2017) 7: 11779. An ultrasonic pulse is then delivered to the optic nerve. ONC-TON is performed by exposing the optic nerve of an adult rat and creating a small opening in the meninges of the nerve, as described in Solomon, A. et al., J. of Neurosci. Methods (1996) 70: 21-25.

A baseline recording of visual function by pattern electroretinogram (PERG) and imaging of the optic disc is performed prior to the administration of the first dose and again prior to performing a method of inducing TON. These results are compared to PERG recordings and optic nerve disc imaging 1 day, 2 days, 3 days, 4 days, 5 days, 6 days, 7 days, 8 days, 9 days, 10 days, 11 days, 12 days, 13 days, 14 days, 3 weeks, 4 weeks 5 weeks, 6 weeks, 7 weeks, 8 weeks, 12 weeks, and 16 weeks following performance of methods to induce TON. Euthanized animals are evaluated for RGC dropout by flat mount. Optic nerves are dissected posteriorly through the bony optic canal, sectioned with a VT-1000s vibratome, and processed for immunohistochemical analysis of inflammatory and gliotic markers.

### Results

It is anticipated that mice pre-treated with 2′6′-Dmt-D-Arg-Phe-Lys-NH₂, Phe-D-Arg-Phe-Lys-NH₂, or D-Arg-2′6′-Dmt-Lys-Phe-NH₂ will delay or prevent the onset TON as compared to untreated and control peptide treated mice.

It is anticipated that mice pre-treated with 2′6′-Dmt-D-Arg-Phe-Lys-NH₂, Phe-D-Arg-Phe-Lys-NH₂, or D-Arg-2′6′-Dmt-Lys-Phe-NH₂ will exhibit healthier PERG recordings and optic nerve disc morphology compared to untreated and control peptide treated mice. It is also anticipated that mice pre-treated with 2′6′-Dmt-D-Arg-Phe-Lys-NH₂, Phe-D-Arg-Phe-Lys-NH₂, or D-Arg-2′6′-Dmt-Lys-Phe-NH₂ will exhibit healthier RGCs compared to untreated and control peptide treated mice.

These results will show that aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) are useful in preventing or delaying the onset of TON. These results will show that aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) are useful for the prevention of TON and/or reducing the risk of developing TON following a traumatic injury.

### Example 4—Sequential Administration of Aromatic-Cationic Peptides with Additional Therapeutic Agent in the Treatment of TON in an Animal Model

This example demonstrates the in vivo efficacy of the sequential administration of aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) with an additional therapeutic agent in treating TON in animal models of the disease.

### Methods

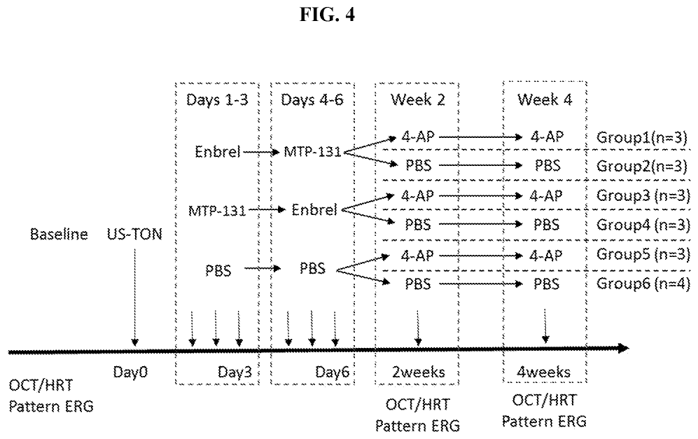

The sequential administration study was conducted as depicted in FIG. 4. A baseline visual function of retinal ganglion cells (RGCs) was obtained using pattern electroretinography (PERG) to record the electrical activity of RGCs, Optical Coherence Tomography (OCT) to evaluate the thickness of the Retinal Nerve Fiber Layer (RNFL), and Heidelberg Retina Topography (HRT) to evaluate subtle changes in the optic nerve caused by trauma. At day zero, US-TON (SI-TON) was induced by a microtip probe sonifier placed on the supraorbital ridge directly above the entrance of the optic nerve into the bony canal, as described in Example 2.

On the first day, six mice were administered either a bolus subcutaneous injection of 3 mg/kg (120 μg per 30 g mouse) Etanercept (Enbrel™, Groups 1 and 2), 10 mg/kg (300 μg per 30 g mouse) D-Arg-2′6′-Dmt-Lys-Phe-NH₂(MTP-131, Groups 3 and 4), or PBS (Groups 5 and 6), followed by daily injections of the drug formulation for a period of 3 days. On the sixth day, Groups 1 and 2 were administered a bolus subcutaneous injection of 10 mg/kg (300 μg per 30 g mouse) D-Arg-2′6′-Dmt-Lys-Phe-NH₂; Groups 3 and 4 were administered a bolus subcutaneous injection of 3 mg/kg (120 μg per 30 g mouse) Etanercept; and Groups 5 and 6 received PBS, followed by daily injections of the drug formulation for three days. Two weeks post-injury, a subset of animals was allowed to progress to a neuropathic state and were administered either 5 mg/kg (150 μg per 30 g mouse) 4-aminopyridine (4-AP, Groups 1, 3, and 5) or PBS (Groups 2, 4, and 6), followed by daily injections of the drug formulation. A subset of mice underwent visual function testing through PERG, and retinal brightfield imaging of optic disc (OCT/HRT). Four weeks post-injury, the subset of animals that was allowed to progress to a neuropathic state underwent visual function testing.

### Results

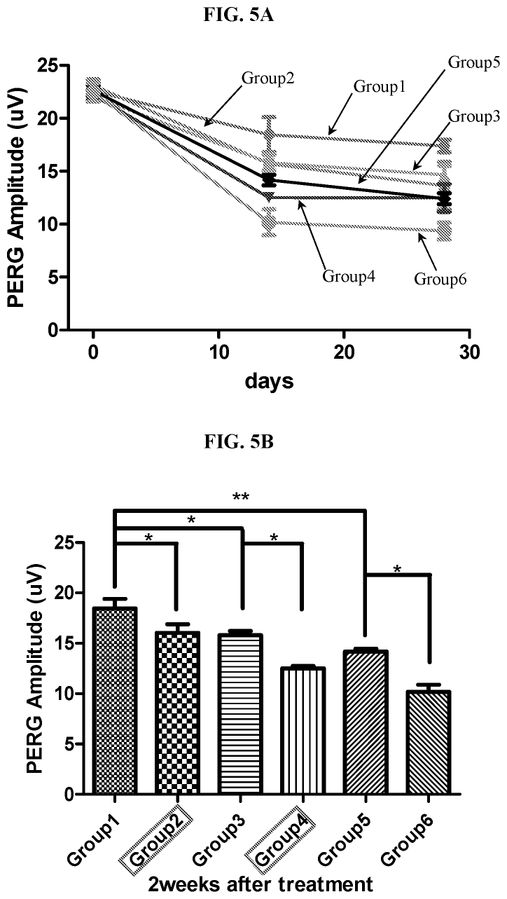

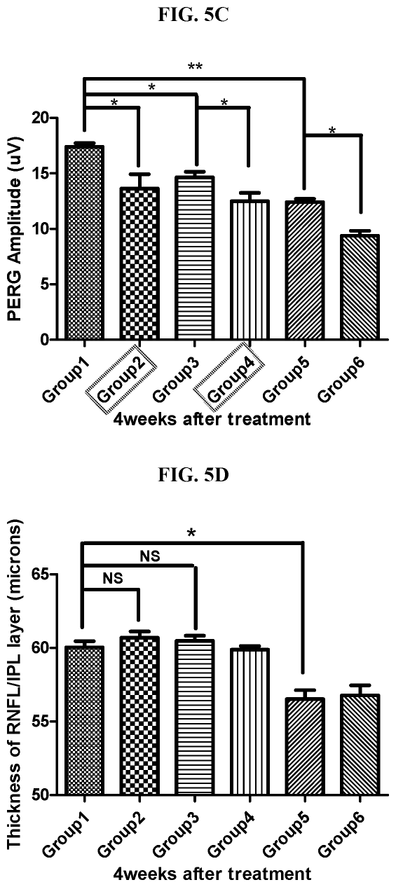

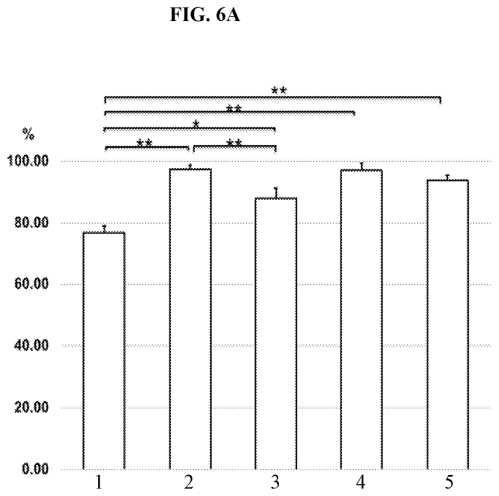

Sequential administration of Etanercept followed by D-Arg-2′6′-Dmt-Lys-Phe-NH₂ improved visual outcomes in TON. At two and four weeks post-injury, animals that initially received subcutaneous injections of Etanercept followed by D-Arg-2′6′-Dmt-Lys-Phe-NH₂ exhibited higher PERG amplitude (Groups 1 and 2) when compared to those that initially received D-Arg-2′6′-Dmt-Lys-Phe-NH₂ followed by Etanercept (FIGS. 5 A-5C). The electrical activity of RGCs (PERG amplitude) at two weeks post treatment was substantially similar as that observed at four weeks post-treatment (FIGS. 5A-5C). However, quantification of the OCT results showed that the RNFL thickness was not significantly different between Groups 1-4 (FIG. 5D). RNFL and IPL layers were thicker in animals administered therapeutic formulations (Groups 1-4) than in animals treated with saline (Groups 5-6; FIG. 5D).

The potassium channel blocker, 4-aminopyridine (4-AP), significantly improved electrophysiological function of RGCs post injury. Addition of 4-AP to any formulations administered to the mice significantly increased RGC electrical activity measured by PERG (FIGS. 5B-5C). The effect of 4-AP was independent of the formulation administration sequence, and the duration of the treatment. PERG amplitudes of Groups 1, 3, and 5 were significantly higher than the PERG amplitudes of Groups 2, 4, and 6 at two and four weeks post treatment. 4-AP did not significantly improve RNFL and IPL thickness as shown by OCT imaging (FIG. 5D).

As demonstrated herein, the sequential administration of D-Arg-2′6′-Dmt-Lys-Phe-NH₂ with an additional therapeutic agent significantly improved the functional benefit of D-Arg-2′6′-Dmt-Lys-Phe-NH₂ for preserving visual function following injury. In particular, the initial administration of the TNFα inhibitor, Etanercept, followed by D-Arg-2′6′-Dmt-Lys-Phe-NH₂ significantly enhanced RGC electrical activity and improved visual function. The addition of the potassium channel blocker, 4-aminopyridine (4-AP), significantly enhanced the electophysiological response in all groups (FIGS. 5B-5C). Accordingly, these results demonstrate that the compositions of the present technology are useful in methods for treating or preventing traumatic optic neuropathy (TON) in a subject in need thereof.

### Example 5—Acute Intravitreal Administration of Aromatic-Cationic Peptides is Safe and Increases RGC Survival Following TON in an Animal Model

This example demonstrates the in vivo safety and efficacy of the sequential acute intravitreal administration of aromatic-cationic peptides, such as 2′6′-Dmt-D-Arg-Phe-Lys-NH₂, Phe-D-Arg-Phe-Lys-NH₂, or D-Arg-2′6′-Dmt-Lys-Phe-NH₂, or a pharmaceutically acceptable salt thereof (such as an mono, bis, or tri-acetate salt, a tartrate salt, a fumarate salt, a mono, bis, or tri-HCl salt, a mono, bis, or tri-tosylate salt, or a mono, bis, or tri-trifluoroacetate salt) with an additional therapeutic agent in treating TON in animal models of the disease.

### Methods

To determine the effect of acute intravitreal administration of aromatic-cationic peptides in combination with sequential administration with an additional therapeutic agent in vivo, SI-TON was induced in 3-month-old C57BL/6 J mice with a Branson Digital Sonifier 450 (Branson) using a 3 mm microtip probe (Branson) in an acoustic soundproof enclosure chamber, as described in Example 2. Fifteen minutes following the induction of TON, 10 animals were administered 1.3 μL of 100 nM D-Arg-2′6′-Dmt-Lys-Phe-NH₂ (MTP-131) intravitreally in the left eye (OS, treated). These mice were then subcutaneously injected with 10 mg/kg Etanercept (Enbrel™) daily for three days followed by subcutaneous injection with 5 mg/kg D-Arg-2′6′-Dmt-Lys-Phe-NH₂ daily for another 3 days. A second group of 10 mice was subcutaneously administered 10 mg/kg Etanercept (Enbrel™) daily for three days followed by 5 mg/kg D-Arg-2′6′-Dmt-Lys-Phe-NH₂ daily for another 3 days. A third group received either 10 mg/kg Etanercept, 5 mg/kg D-Arg-2′6′-Dmt-Lys-Phe-NH₂, or PBS daily for six days. Four weeks after treatment, mice were euthanized. Optic nerves were dissected posteriorly through the bony optic canal, sectioned with a VT-1000s vibratome, and processed for immunohistochemical analysis of inflammatory and gliotic markers. RGCs survival was assayed using immunohistochemical staining for the neuronal marker Tubulin Beta 3 Class III (TUBB3) followed by a manual count of remaining RGC somas per unit area by 2 independent blinded investigators. RGC survival is reported as a percentage of RGC counts per unit area relative to counts obtained from uninjured wildtype controls (naïve controls).

To determine the safety of acute intravitreal administration of aromatic-cationic peptides in vivo, SI-TON was induced in 3-month-old C57BL/6 J mice with a Branson Digital Sonifier 450 (Branson) by a 3 mm microtip probe (Branson) in an acoustic soundproof enclosure chamber, as described in Example 2. Fifteen minutes following the induction of TON, a group of ten mice were administered 1.3 μL of 100 nM D-Arg-2′6′-Dmt-Lys-Phe-NH₂ (MTP-131) intravitreally in the left eye (OS, treated). A different subset of mice was administered control PBS solution intravitreally in the left eye (OS, treated) followed by subcutaneous injection with 5 mg/kg D-Arg-2′6′-Dmt-Lys-Phe-NH₂ fifteen minutes post-injury. A baseline recording of the mice visual function was recorded using electrophysiological recording of RGCs through PERG prior to SI-TON induction and the functional visual outcome of the mice was tested again 4 weeks post-injury.

### Results

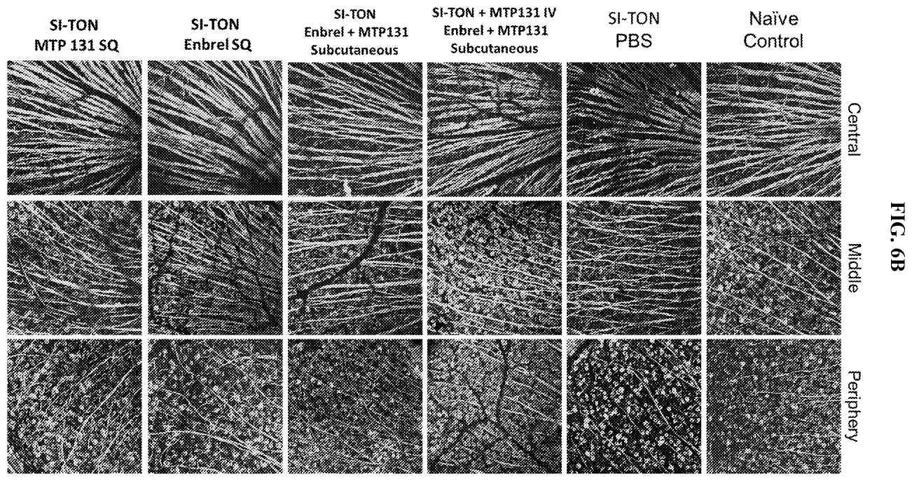

Acute intravitreal administration of D-Arg-2′6′-Dmt-Lys-Phe-NH₂ promotes RGCs survival in TON. Immediate intravitreal administration of D-Arg-2′6′-Dmt-Lys-Phe-NH₂ following TON induction coupled with subcutaneous sequential injection with Etanercept and D-Arg-2′6′-Dmt-Lys-Phe-NH₂ significantly protected RGCs from cell death (FIG. 6A). Two weeks post-injury, approximately 80% RGCs survived SI-TON, while approximately 90%-100% of RGCs survived when D-Arg-2′6′-Dmt-Lys-Phe-NH₂ (p<0.01, n=5) and Etanercept ((p<0.01, n=5) were subcutaneously injected alone (FIG. 6A).

Acute intravitreal injection of D-Arg-2′6′-Dmt-Lys-Phe-NH₂ protects nerve fiber bundle morphology in SI-TON. Immunihistochemical images demonstrate preservation of nerve fiber bundle caliber and morphology throughout the retina in D-Arg-2′6′-Dmt-Lys-Phe-NH₂-treated animals following SI-TON injury (FIG. 6B). In PBS-treated animals, nerve fiber bundle staining demonstrates loss of RGC axonal projections, represented by decreased immunofluorescence intensity and fiber caliber, as well as disruption of bundle morphology and distribution.

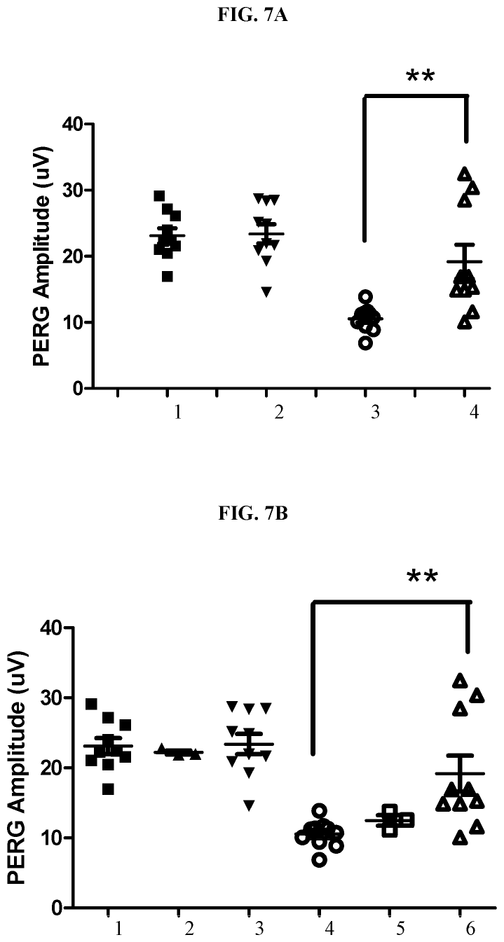

Acute intravitreal injection of D-Arg-2′6′-Dmt-Lys-Phe-NH₂ is safe and effective. The baseline electrophysiological recording of RGCs was substantially similar in control and D-Arg-2′6′-Dmt-Lys-Phe-NH₂-treated animals (FIG. 7A). In non-treated animals, SI-TON induction reduced RGC electrical activity by about 50%. In animals treated with acute intravitreal injection of 100 nM D-Arg-2′6′-Dmt-Lys-Phe-NH₂₁₅ minutes post injury, the traumatic effect of SI-TON on RGCs was significantly reduced when compared to non-injected animals (FIG. 7A).

The baseline electrophysiological recording of RGCs were substantially similar in control and D-Arg-2′6′-Dmt-Lys-Phe-NH₂-treated animals (FIG. 7B). Acute intravitreal injection of D-Arg-2′6′-Dmt-Lys-Phe-NH₂ after injury augmented the visual outcome (PERG) effect of the drug beyond subcutaneous administration alone (FIG. 7B).

The results demonstrate that intravitreal injection of D-Arg-2′6′-Dmt-Lys-Phe-NH₂ coupled with sequential subcutaneous injection of Etanercept and D-Arg-2′6′-Dmt-Lys-Phe-NH₂ was more effective than subcutaneous injection alone at preventing the effect of SI-TON on RGC survival. Furthermore, direct application of D-Arg-2′6′-Dmt-Lys-Phe-NH₂ to the eye was safe (did not show evidence of toxicity), and improved visual outcomes beyond subcutaneous administration alone, as shown by PERG recording. Accordingly, these results demonstrate that the compositions of the present technology are useful in methods for treating or preventing traumatic optic neuropathy (TON) in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a nonlimiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

What is claimed is:

1. A method for treating or preventing traumatic optic neuropathy (TON) in a subject in need thereof, comprising administering to the subject a therapeutically effective amount of the peptide D-Arg-2′,6′-Dmt-Lys-Phe-NH₂, or a pharmaceutically acceptable salt thereof.

2. The method of claim 1, wherein the subject has been diagnosed as having TON.

3. The method of any one of the previous claims, wherein the TON is caused by direct injury or indirect injury to the subject.

4. The method of claim 3, wherein the direct or indirect injury is selected from the group consisting of intraorbital injury, intracanalicular injury, intracranial injury, and an injury to the subject's optic nerve.

5. The method of claim 3 or 4, wherein the peptide is administered prior to injury.

6. The method of claim 3 or 4, wherein the peptide is administered immediately following injury.

7. The method of claim 3 or 4, wherein the peptide is administered about 2 hours or less, about 6 hours or less, about 12 hours or less, or about 24 hours or less following the injury.

8. The method of any one of the previous claims, wherein the peptide is administered daily for 2 weeks or more.

9. The method of any one of the previous claims, wherein the peptide is administered daily for 12 weeks or more.

10. The method of any one of the previous claims, wherein the treating or preventing comprises the treatment or prevention of one or more signs or symptoms of TON comprising one or more of vision loss, blurred vision, scotoma, decreased color sensation, uveitis, optic neuritis, eye pain, optic nerve avulsion, optic nerve transection, optic nerve sheath hemorrhage, orbital hemorrhage, choroidal rupture, and commotio retinae.

11. The method of any one of the previous claims, wherein the subject is a mammal.

12. The method of claim 11, wherein the mammalian subject is a human.

13. The method of any one of the previous claims, wherein the peptide is administered orally, topically, intranasally, systemically, intravenously, subcutaneously, intraperitoneally, intradermally, intraocularly, ophthalmically, iontophoretically, transmucosally, intravitreally, or intramuscularly.

14. The method of any one of the previous claims, further comprising separately, sequentially, or simultaneously administering an additional treatment to the subject.

15. The method of claim 14, wherein the additional treatment comprises administration of a therapeutic agent.

16. The method of claim 15, wherein the therapeutic agent is selected from the group consisting of: TNFα inhibitor, corticosteroid, IL-1R antagonist, resveratrol, potassium channel blocker, and necrostatin-1.

17. The method of claim 15, wherein the TNFα inhibitor is etanercept.

18. The method of claim 14, wherein the additional treatment comprises reducing the core temperature of the subject.

19. The method of claim 18, the core temperature of the subject is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%.

20. The method of claim 18 or 19, wherein hypothermia is induced in the subject.

21. The method of any one of claims 14-20, wherein the combination of peptide and an additional therapeutic treatment has a synergistic effect in the prevention or treatment of TON.

22. The method of any one of the previous claims, wherein the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride (“mono-HCl”) salt, a bis-hydrochloride (“bis-HCl”) salt, a tri-hydrochloride (“tri-HCl”) salt, a mono-tosylate salt, a bis-tosylate salt, or a tri-tosylate salt.

23. The method of any one of claims 1-21, wherein the pharmaceutically acceptable salt of the peptide that is formulated for administering to the subject is as a tri-HCl salt, a bis-HCl salt, or a mono-HCl salt.

24. A method for improving visual function in a subject having traumatic optic neuropathy (TON), the method comprising administering to the subject a therapeutically effective amount of the peptide D-Arg-2′,6′-Dmt-Lys-Phe-NH₂, or a pharmaceutically acceptable salt thereof.

25. The method of claim 24, wherein the subject has experienced a direct injury or an indirect injury.

26. The method of claim 25, wherein the direct or indirect injury is selected from the group consisting of intraorbital injury, intracanalicular injury, intracranial injury, and an injury to the subject's optic nerve.

27. The method of claim 25 or 26, wherein the peptide is administered immediately following injury.

28. The method of claim 25 or 26, wherein the peptide is administered about 2 hours or less, about 6 hours or less, about 12 hours or less, or about 24 hours or less following the injury.

29. The method of any one of claims 24-28, wherein the peptide is administered daily for 2 weeks or more.

30. The method of any one of claims 24-28, wherein the peptide is administered daily for 12 weeks or more.

31. The method of any one of claims 24-30, wherein the visual function is assessed by one or more of pattern electroretinography (PERG), detection of best corrected visual acuity (BVCA), electroretinography (ERG), and optical coherence tomography (OCT).

32. The method of any one of claims 24-31, wherein the improved visual function comprises improvements in any one or more of visual acuity, BVCA, thickness of the retina as detected by OCT, PERG amplitude, ERG amplitude, ERG latency, vision loss, blurred vision, scotoma, decreased color sensation, uveitis, optic neuritis, eye pain, optic nerve avulsion, optic nerve transection, optic nerve sheath hemorrhage, orbital hemorrhage, choroidal rupture, and commotio retinae compared to an untreated control.

33. The method of any one of claims 24-32, wherein the subject is a mammal.

34. The method of claim 33, wherein the mammalian subject is a human.

35. The method of any one of claims 24-34, wherein the peptide is administered orally, topically, intranasally, systemically, intravenously, subcutaneously, intraperitoneally, intradermally, intraocularly, ophthalmically, iontophoretically, transmucosally, intravitreally, or intramuscularly.

36. The method of any one of claims 24-35, further comprising separately, sequentially, or simultaneously administering an additional treatment to the subject.

37. The method of claim 36, wherein the additional treatment comprises administration of a therapeutic agent.

38. The method of claim 37, wherein the therapeutic agent is selected from the group consisting of: TNFα inhibitor, corticosteroid, IL-1R antagonist, resveratrol, potassium channel blocker, and necrostatin-1.

39. The method of claim 38, wherein the TNFα inhibitor is etanercept.

40. The method of claim 36, wherein the additional treatment comprises reducing the core temperature of the subject.

41. The method of claim 40, the core temperature of the subject is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%.

42. The method of claim 41 or 42, wherein hypothermia is induced in the subject.

43. The method of any one of claims 36-42, wherein the combination of peptide and an additional treatment has a synergistic effect in improving visual function.

44. The method of any one of claims 24-43, wherein the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride (“mono-HCl”) salt, a bis-hydrochloride (“bis-HCl”) salt, a tri-hydrochloride (“tri-HCl”) salt, a mono-tosylate salt, a bis-tosylate salt, or a tri-tosylate salt.

45. The method of any one of claims 24-43, wherein the pharmaceutically acceptable salt of the peptide that is formulated for administering to the subject is a tri-HCl salt, a bis-HCl salt, or a mono-HCl salt.

46. A method of promoting retinal ganglion cell (RGC) survival or increasing neurite outgrowth of an RGC comprising contacting an RGC with an effective amount of the peptide D-Arg-2′,6′-Dmt-Lys-Phe-NH₂, or a pharmaceutically acceptable salt thereof.

47. The method of claim 46, wherein the RGC is in vitro.

48. The method of claim 46, wherein the RGC is in a subject with TON.

49. The method of claim 48, wherein the subject is a mammal.

50. The method of claim 49, wherein the mammalian subject is a human.

51. The method of any one of claims 46-50, further comprising separately, sequentially, or simultaneously administering an additional treatment to the subject.

52. The method of claim 51, wherein the additional treatment comprises administration of a therapeutic agent.

53. The method of claim 52, wherein the therapeutic agent is selected from the group consisting of: TNFα inhibitor, corticosteroid, IL-1R antagonist, resveratrol, potassium channel blocker, and necrostatin-1.

54. The method of claim 53, wherein the TNFα inhibitor is etanercept.

55. The method of claim 54, wherein the additional treatment comprises reducing the core temperature of the subject.

56. The method of claim 55, the core temperature of the subject is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%.

57. The method of claim 54 or 55, wherein hypothermia is induced in the subject.

58. The method of any one of claims 51-57, wherein the combination of peptide and an additional treatment has a synergistic effect in in promoting RGC survival or increasing neurite outgrowth of an RGC.

59. The method of any one of claims 46-58, wherein the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride (“mono-HCl”) salt, a bis-hydrochloride (“bis-HCl”) salt, a tri-hydrochloride (“tri-HCl”) salt, a mono-tosylate salt, a bis-tosylate salt, or a tri-tosylate salt.

60. The method of any one of claims 46-58, wherein the pharmaceutically acceptable salt of the peptide that is formulated for administering to the subject is a tri-HCl salt, a bis-HCl salt, or a mono-HCl salt.

61. Use of a composition in the preparation of a medicament for treating or preventing traumatic optic neuropathy (TON) in a subject in need thereof, wherein the composition comprises a therapeutically effective amount of the peptide D-Arg-2′,6′-Dmt-Lys-Phe-NH₂, or a pharmaceutically acceptable salt thereof.

62. The use of claim 61, wherein the subject has been diagnosed as having TON.

63. The use of claim 61 or 62, wherein the TON is caused by direct injury or indirect injury to the subject.

64. The use of claim 63, wherein the direct or indirect injury is selected from the group consisting of intraorbital injury, intracanalicular injury, intracranial injury, and an injury to the subject's optic nerve.

65. The use of claim 63 or 64, wherein the peptide is intended to be administered prior to injury.

66. The use of claim 63 or 64, wherein the peptide is intended to be administered immediately following injury.

67. The use of claim 63 or 64, wherein the peptide is intended to be administered about 2 hours or less, about 6 hours or less, about 12 hours or less, or about 24 hours or less following the injury.

68. The use of any one of claims 61-67, wherein the peptide is intended to be administered daily for 2 weeks or more.

69. The use of any one of claims 61-67, wherein the peptide is intended to be administered daily for 12 weeks or more.

70. The use of any one of claims 61-69, wherein the treating or preventing comprises the treatment or prevention of one or more signs or symptoms of TON comprising one or more of vision loss, blurred vision, scotoma, decreased color sensation, uveitis, optic neuritis, eye pain, optic nerve avulsion, optic nerve transection, optic nerve sheath hemorrhage, orbital hemorrhage, choroidal rupture, and commotio retinae.

71. The use of any one of claims 61-70, wherein the subject is a mammal.

72. The use of claim 71, wherein the mammalian subject is a human.

73. The use of any one of claims 61-72, wherein the peptide is formulated for administration orally, topically, intranasally, systemically, intravenously, subcutaneously, intraperitoneally, intradermally, intraocularly, ophthalmically, iontophoretically, transmucosally, intravitreally, or intramuscularly.

74. The use of any one of claims 61-73, wherein the peptide is intended to be separately, sequentially, or simultaneously used with an additional treatment.

75. The use of claim 74, wherein the additional treatment comprises use of a therapeutic agent.

76. The use of claim 75, wherein the therapeutic agent is selected from the group consisting of: TNFα inhibitor, corticosteroid, IL-1R antagonist, resveratrol, potassium channel blocker, and necrostatin-1.

77. The use of claim 76, wherein the TNFα inhibitor is etanercept.

78. The use of claim 74, wherein the additional treatment comprises reducing the core temperature of the subject.

79. The use of claim 78, the core temperature of the subject is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%.

80. The use of claim 78 or 79, wherein hypothermia is induced in the subject.

81. The use of any one of claims 74-80, wherein the combination of peptide and an additional treatment has a synergistic effect in the prevention or treatment of TON.

82. The use of any one of claims 61-81, wherein the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride (“mono-HCl”) salt, a bis-hydrochloride (“bis-HCl”) salt, a tri-hydrochloride (“tri-HCl”) salt, a mono-tosylate salt, a bis-tosylate salt, or a tri-tosylate salt.

83. The use of any one of claims 61-81, wherein the pharmaceutically acceptable salt of the peptide that is formulated for administering to the subject is a tri-HCl salt, a bis-HCl salt, or a mono-HCl salt.

84. Use of a composition in the preparation of a medicament for improving visual function in a subject having traumatic optic neuropathy (TON), wherein the composition comprises a therapeutically effective amount of the peptide D-Arg-2′,6′-Dmt-Lys-Phe-NH₂, or a pharmaceutically acceptable salt thereof.

85. The use of claim 84, wherein the subject has experienced a direct injury or an indirect injury.

86. The use of claim 85, wherein the direct or indirect injury is selected from the group consisting of intraorbital injury, intracanalicular injury, intracranial injury, and an injury to the subject's optic nerve.

87. The use of claim 85 or 86, wherein the peptide is intended to be administered immediately following injury.

88. The use of claim 85 or 86, wherein the peptide is intended to be administered about 2 hours or less, about 6 hours or less, about 12 hours or less, or about 24 hours or less following the injury.

89. The use of any one of claims 84-88, wherein the peptide is intended to be administered daily for 2 weeks or more.

90. The use of any one of claims 84-88, wherein the peptide is intended to be administered daily for 12 weeks or more.

91. The use of any one of claims 84-90, wherein the visual function is assessed by one or more of pattern electroretinography (PERG), detection of best corrected visual acuity (BVCA), electroretinography (ERG), and optical coherence tomography (OCT).

92. The use of any one of claims 84-91, wherein the improved visual function comprises improvements in any one or more of visual acuity, BVCA, thickness of the retina as detected by OCT, PERG amplitude, ERG amplitude, ERG latency, vision loss, blurred vision, scotoma, decreased color sensation, uveitis, optic neuritis, eye pain, optic nerve avulsion, optic nerve transection, optic nerve sheath hemorrhage, orbital hemorrhage, choroidal rupture, and commotio retinae compared to an untreated control.

93. The use of any one of claims 84-92, wherein the subject is a mammal.

94. The use of claim 93, wherein the mammalian subject is a human.

95. The use of any one of claims 84-94, wherein the peptide is intended to be administered orally, topically, intranasally, systemically, intravenously, subcutaneously, intraperitoneally, intradermally, intraocularly, ophthalmically, iontophoretically, transmucosally, intravitreally, or intramuscularly.

96. The use of any one of claims 84-95, wherein the peptide is intended to be separately, sequentially, or simultaneously used with an additional treatment.

97. The use of claim 96, wherein the additional treatment comprises use of a therapeutic agent.

98. The use of claim 97, wherein the therapeutic agent is selected from the group consisting of: TNFα inhibitor, corticosteroid, IL-1R antagonist, resveratrol, potassium channel blocker, and necrostatin-1.

99. The use of claim 98, wherein the TNFα inhibitor is etanercept.

100. The use of claim 96, wherein the additional treatment comprises reducing the core temperature of the subject.

101. The use of claim 100, the core temperature of the subject is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%.

102. The use of claim 100 or 101, wherein hypothermia is induced in the subject.

103. The use of any one of claims 96-102, wherein the combination of peptide and an additional treatment has a synergistic effect in improving visual function.

104. The use of any one of claims 84-103, wherein the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride (“mono-HCl”) salt, a bis-hydrochloride (“bis-HCl”) salt, a tri-hydrochloride (“tri-HCl”) salt, a mono-tosylate salt, a bis-tosylate salt, or a tri-tosylate salt.

105. The use of any one of claims 84-103, wherein the pharmaceutically acceptable salt of the peptide that is formulated for administering to the subject is a tri-HCl salt, a bis-HCl salt, or a mono-HCl salt.

106. Use of a composition in the preparation of a medicament for promoting retinal ganglion cell (RGC) survival or increasing neurite outgrowth of an RGC, wherein the composition comprises an effective amount of the peptide D-Arg-2′,6′-Dmt-Lys-Phe-NH₂, or a pharmaceutically acceptable salt thereof.

107. The use of claim 106, wherein the RGC is in vitro.

108. The use of claim 106, wherein the RGC is in a subject with TON.

109. The use of claim 108, wherein the subject is a mammal.

110. The use of claim 109, wherein the mammalian subject is a human.

111. The use of any one of claims 106-110, wherein the peptide is intended to be separately, sequentially, or simultaneously used an additional treatment.

112. The use of claim 111, wherein the additional treatment comprises use of a therapeutic agent.

113. The use of claim 112, wherein the therapeutic agent is selected from the group consisting of: TNFα inhibitor, corticosteroid, IL-1R antagonist, resveratrol, potassium channel blocker, and necrostatin-1.

114. The use of claim 113, wherein the TNFα inhibitor is etanercept.

115. The use of claim 114, wherein the additional treatment comprises reducing core temperature.

116. The use of claim 115, the core temperature is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%.

117. The use of claim 114 or 115, wherein hypothermia is induced.

118. The use of any one of claims 111-117, wherein the combination of peptide and an additional treatment has a synergistic effect in promoting RGC survival or increasing neurite outgrowth of an RGC.

119. The use of any one of claims 106-118, wherein the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride (“mono-HCl”) salt, a bis-hydrochloride (“bis-HCl”) salt, a tri-hydrochloride (“tri-HCl”) salt, a mono-tosylate salt, a bis-tosylate salt, or a tri-tosylate salt.

120. The use of any one of claims 106-118, wherein the pharmaceutically acceptable salt of the peptide that is formulated for administering to the subject is a tri-HCl salt, a bis-HCl salt, or a mono-HCl salt.

121. A peptide D-Arg-2′,6′-Dmt-Lys-Phe-NH₂, or a pharmaceutically acceptable salt thereof, for use in treating or preventing traumatic optic neuropathy (TON) in a subject in need thereof.

122. The peptide of claim 121, for use wherein the subject has been diagnosed as having TON.

123. The peptide of claim 121 or 122, for use wherein the TON is caused by direct injury or indirect injury to the subject.

124. The peptide of claim 123, for use wherein the direct or indirect injury is selected from the group consisting of intraorbital injury, intracanalicular injury, intracranial injury, and an injury to the subject's optic nerve.

125. The peptide of claim 123 or 124, for use wherein the peptide is intended to be administered prior to injury.

126. The peptide of claim 123 or 124, for use wherein the peptide is intended to be administered immediately following injury.

127. The peptide of claim 123 or 124, for use wherein the peptide is intended to be administered about 2 hours or less, about 6 hours or less, about 12 hours or less, or about 24 hours or less following the injury.

128. The peptide of any one of claims 121-127, for use wherein the peptide is intended to be administered daily for 2 weeks or more.

129. The peptide of any one of claims 121-127, for use wherein the peptide is intended to be administered daily for 12 weeks or more.

130. The peptide of any one of claims 121-129, for use wherein the treating or preventing comprises the treatment or prevention of one or more signs or symptoms of TON comprising one or more of vision loss, blurred vision, scotoma, decreased color sensation, uveitis, optic neuritis, eye pain, optic nerve avulsion, optic nerve transection, optic nerve sheath hemorrhage, orbital hemorrhage, choroidal rupture, and commotio retinae.

131. The peptide of any one of claims 121-130, for use wherein the subject is a mammal.

132. The peptide of claim 131, for use wherein the mammalian subject is a human.

133. The peptide of any one of claims 121-132, for use wherein the peptide is formulated for administration orally, topically, intranasally, systemically, intravenously, subcutaneously, intraperitoneally, intradermally, intraocularly, ophthalmically, iontophoretically, transmucosally, intravitreally, or intramuscularly.

134. The peptide of any one of claims 121-133, for use wherein the peptide is intended to be separately, sequentially, or simultaneously used with an additional treatment.

135. The peptide of claim 134, for use wherein the additional treatment comprises use of a therapeutic agent.

136. The peptide of claim 135, for use wherein the therapeutic agent is selected from the group consisting of: TNFα inhibitor, corticosteroid, IL-1R antagonist, resveratrol, potassium channel blocker, and necrostatin-1.

137. The peptide of claim 136, for use wherein the TNFα inhibitor is etanercept.

138. The peptide of claim 134, for use wherein the additional treatment comprises reducing the core temperature of the subject.

139. The peptide of claim 138, for use wherein the core temperature of the subject is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%.

140. The peptide of claim 138 or 139, for use wherein hypothermia is induced in the subject.

141. The peptide of any one of claims 134-140, for use wherein the combination of peptide and an additional treatment has a synergistic effect in the prevention or treatment of TON.

142. The peptide of any one of claims 121-141, for use wherein the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride (“mono-HCl”) salt, a bis-hydrochloride (“bis-HCl”) salt, a tri-hydrochloride (“tri-HCl”) salt, a mono-tosylate salt, a bis-tosylate salt, or a tri-tosylate salt.

143. The peptide of any one of claims 121-141, for use wherein the pharmaceutically acceptable salt of the peptide that is formulated for administering to the subject is a tri-HCl salt, a bis-HCl salt, or a mono-HCl salt.

144. A peptide D-Arg-2′,6′-Dmt-Lys-Phe-NH₂, or a pharmaceutically acceptable salt thereof, for use in improving visual function in a subject having traumatic optic neuropathy (TON).

145. The peptide of claim 144, for use wherein the subject has experienced a direct injury or an indirect injury.

146. The peptide of claim 145, for use wherein the direct or indirect injury is selected from the group consisting of intraorbital injury, intracanalicular injury, intracranial injury, and an injury to the subject's optic nerve.

147. The peptide of claim 145 or 146, for use wherein the peptide is intended to be administered immediately following injury.

148. The peptide of claim 145 or 146, for use wherein the peptide is intended to be administered about 2 hours or less, about 6 hours or less, about 12 hours or less, or about 24 hours or less following the injury.

149. The peptide of any one of claims 144-148, for use wherein the peptide is intended to be administered daily for 2 weeks or more.

150. The peptide of any one of claims 144-148, for use wherein the peptide is intended to be administered daily for 12 weeks or more.

151. The peptide of any one of claims 144-150, for use wherein the visual function is assessed by one or more of pattern electroretinography (PERG), detection of best corrected visual acuity (BVCA), electroretinography (ERG), and optical coherence tomography (OCT).

152. The peptide of any one of claims 144-151, for use wherein the improved visual function comprises improvements in any one or more of visual acuity, BVCA, thickness of the retina as detected by OCT, PERG amplitude, ERG amplitude, ERG latency, vision loss, blurred vision, scotoma, decreased color sensation, uveitis, optic neuritis, eye pain, optic nerve avulsion, optic nerve transection, optic nerve sheath hemorrhage, orbital hemorrhage, choroidal rupture, and commotio retinae compared to an untreated control.

153. The peptide of any one of claims 144-152, for use wherein the subject is a mammal.

154. The peptide of claim 153, wherein the mammalian subject is a human.

155. The peptide of any one of claims 144-154, for use wherein the peptide is intended to be administered orally, topically, intranasally, systemically, intravenously, subcutaneously, intraperitoneally, intradermally, intraocularly, ophthalmically, iontophoretically, transmucosally, intravitreally, or intramuscularly.

156. The peptide of any one of claims 144-155, for use wherein the peptide is intended to be separately, sequentially, or simultaneously used with an additional treatment.

157. The peptide of claim 156, for use wherein the additional treatment comprises use of a therapeutic agent.

158. The peptide of claim 157, for use wherein the therapeutic agent is selected from the group consisting of: TNFα inhibitor, corticosteroid, IL-1R antagonist, resveratrol, potassium channel blocker, and necrostatin-1.

159. The peptide of claim 158, for use wherein the TNFα inhibitor is etanercept.

160. The peptide of claim 156, for use wherein the additional treatment comprises reducing the core temperature of the subject.

161. The peptide of claim 160, for use wherein the core temperature of the subject is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%.

162. The peptide of claim 160 or 161, for use wherein hypothermia is induced in the subject.

163. The peptide of any one of claims 156 to 162, for use wherein the combination of peptide and an additional treatment has a synergistic effect in improving visual function.

164. The peptide of any one of claims 144-163, for use wherein the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride (“mono-HCl”) salt, a bis-hydrochloride (“bis-HCl”) salt, a tri-hydrochloride (“tri-HCl”) salt, a mono-tosylate salt, a bis-tosylate salt, or a tri-tosylate salt.

165. The peptide of any one of claims 144-163, for use wherein the pharmaceutically acceptable salt of the peptide that is formulated for administering to the subject is a tri-HCl salt, a bis-HCl salt, or a mono-HCl salt.

166. A peptide D-Arg-2′,6′-Dmt-Lys-Phe-NH₂, or a pharmaceutically acceptable salt thereof, for use in promoting retinal ganglion cell (RGC) survival or increasing neurite outgrowth of an RGC.

167. The peptide of claim 166, for use wherein the RGC is in vitro.

168. The peptide of claim 166, for use wherein the RGC is in a subject with TON.

169. The peptide of claim 168, for use wherein the subject is a mammal.

170. The peptide of claim 169, for use wherein the mammalian subject is a human.

171. The peptide of any one of claims 166-170, for use wherein the peptide is intended to be separately, sequentially, or simultaneously used an additional treatment.

172. The peptide of claim 171, for use wherein the additional treatment comprises use of a therapeutic agent.

173. The peptide of claim 172, for use wherein the therapeutic agent is selected from the group consisting of: TNFα inhibitor, corticosteroid, IL-1R antagonist, resveratrol, potassium channel blocker, and necrostatin-1.

174. The peptide of claim 173, for use wherein the TNFα inhibitor is etanercept.

175. The peptide of claim 174, for use wherein the additional treatment comprises reducing core temperature.

176. The peptide of claim 175, the core temperature is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%.

177. The peptide of claim 175 or 176, for use wherein hypothermia is induced.

178. The peptide of any one of claims 171-177, for use wherein the combination of peptide and an additional treatment has a synergistic effect in in promoting RGC survival or increasing neurite outgrowth of an RGC.

179. The peptide of any one of claims 166-178, for use wherein the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride (“mono-HCl”) salt, a bis-hydrochloride (“bis-HCl”) salt, a tri-hydrochloride (“tri-HCl”) salt, a mono-tosylate salt, a bis-tosylate salt, or a tri-tosylate salt.

180. The peptide of any one of claims 166-178, for use wherein the pharmaceutically acceptable salt of the peptide that is formulated for administering to the subject is a tri-HCl salt, a bis-HCl salt, or a mono-HCl salt.

181. A method for reducing the risk of TON in a subject that has experienced a traumatic injury, the method comprising administering to the subject a therapeutically effective amount of the peptide D-Arg-2′,6′-Dmt-Lys-Phe-NH₂, or a pharmaceutically acceptable salt thereof.

182. The method of claim 181, wherein the traumatic injury is a direct injury or an indirect injury.

183. The method of claim 182, wherein the direct or indirect injury is selected from the group consisting of intraorbital injury, intracanalicular injury, intracranial injury, and an injury to the subject's optic nerve.

184. The method of any one of claims 181-183, wherein the peptide is administered immediately following the traumatic injury.

185. The method of any one of claims 181-183, wherein the peptide is administered about 2 hours or less, about 6 hours or less, about 12 hours or less, or about 24 hours or less following the traumatic injury.

186. The method of any one of claims 181-185, wherein the peptide is administered daily for 2 weeks or more.

187. The method of any one of claims 181-185, wherein the peptide is administered daily for 12 weeks or more.

188. The method of any one of claims 181-187, wherein the subject is a mammal.

189. The method of claim 188, wherein the mammalian subject is a human.

190. The method of any one of claims 181-189, wherein the peptide is administered orally, topically, intranasally, systemically, intravenously, subcutaneously, intraperitoneally, intradermally, intraocularly, ophthalmically, iontophoretically, transmucosally, intravitreally, or intramuscularly.

191. The method of any one of claims 181-190, further comprising separately, sequentially, or simultaneously administering an additional treatment to the subject.

192. The method of claim 191, wherein the additional treatment comprises administration of a therapeutic agent.

193. The method of claim 192, wherein the therapeutic agent is selected from the group consisting of: TNFα inhibitor, corticosteroid, IL-1R antagonist, resveratrol, potassium channel blocker, and necrostatin-1.

194. The method of claim 193, wherein the TNFα inhibitor is etanercept.

195. The method of claim 191, wherein the additional treatment comprises reducing the core temperature of the subject.

196. The method of claim 195, the core temperature of the subject is reduced by about 2%, about 3%, about 4%, about 5%, about 6%, about 7%, about 8%, about 9%, about 10%, about 15%, about 20%, about 25%, about 30%, about 35%, about 40%, about 45%, or about 50%.

197. The method of claim 195 or 196, wherein hypothermia is induced in the subject.

198. The method of any one of claims 191-197, wherein the combination of peptide and an additional treatment has a synergistic effect in the prevention or treatment of TON.

199. The method of any one of claims 181-198, wherein the pharmaceutically acceptable salt comprises a mono-acetate salt, a bis-acetate salt, a tri-acetate salt, a tartrate salt, a mono-trifluoroacetate salt, a bis-trifluoroacetate salt, a tri-trifluoroacetate salt, a mono-hydrochloride (“mono-HCl”) salt, a bis-hydrochloride (“bis-HCl”) salt, a tri-hydrochloride (“tri-HCl”) salt, a mono-tosylate salt, a bis-tosylate salt, or a tri-tosylate salt.

200. The method of any one of claims 181-198, wherein the pharmaceutically acceptable salt of the peptide that is formulated for administering to the subject is a tri-HCl salt, a bis-HCl salt, or a mono-HCl salt.

201. The method of any one of claim 16, 38, 53, or 193, or the use of any one of claim 76, 98, or 113, or the peptide of any one of claim 136, 158, or 173, wherein the potassium channel blocker is 4-aminopyridine (4-AP).
